SEQUENCE LISTING (297 sequences)

SEQ ID NO: 1 - DNA, 42 bp, Artificial Sequence
  Canonical reference sequence
attgaggatgtaggactcccagctaaaactgccttctgccca

SEQ ID NO: 2 - DNA, 42 bp, Artificial Sequence
  Variant
cttgaggatgtaggactcccagctaaaactgccttctgccca

SEQ ID NO: 3 - DNA, 42 bp, Artificial Sequence
  variant
tttgaggatgtaggactcccagctaaaactgccttctgccca

SEQ ID NO: 4 - DNA, 42 bp, Artificial Sequence
  variant
gttgaggatgtaggactcccagctaaaactgccttctgccca

SEQ ID NO: 5 - DNA, 42 bp, Artificial Sequence
  variant
aatgaggatgtaggactcccagctaaaactgccttctgccca

SEQ ID NO: 6 - DNA, 42 bp, Artificial Sequence
  variant
actgaggatgtaggactcccagctaaaactgccttctgccca

SEQ ID NO: 7 - DNA, 42 bp, Artificial Sequence
  variant
agtgaggatgtaggactcccagctaaaactgccttctgccca

SEQ ID NO: 8 - DNA, 42 bp, Artificial Sequence
  variant
atagaggatgtaggactcccagctaaaactgccttctgccca

SEQ ID NO: 9 - DNA, 42 bp, Artificial Sequence
  variant
atcgaggatgtaggactcccagctaaaactgccttctgccca

SEQ ID NO: 10 - DNA, 42 bp, Artificial Sequence
  variant
atggaggatgtaggactcccagctaaaactgccttctgccca

SEQ ID NO: 11 - DNA, 42 bp, Artificial Sequence
  variant
attaaggatgtaggactcccagctaaaactgccttctgccca

SEQ ID NO: 12 - DNA, 42 bp, Artificial Sequence
  variant
attcaggatgtaggactcccagctaaaactgccttctgccca

SEQ ID NO: 13 - DNA, 42 bp, Artificial Sequence
  variant
atttaggatgtaggactcccagctaaaactgccttctgccca

SEQ ID NO: 14 - DNA, 42 bp, Artificial Sequence; identical to SEQ ID NO: 2
  variant

SEQ ID NO: 15 - DNA, 42 bp, Artificial Sequence
  variant
catgaggatgtaggactcccagctaaaactgccttctgccca

SEQ ID NO: 16 - DNA, 42 bp, Artificial Sequence
  variant
cctgaggatgtaggactcccagctaaaactgccttctgccca

SEQ ID NO: 17 - DNA, 42 bp, Artificial Sequence
  variant
cgtgaggatgtaggactcccagctaaaactgccttctgccca

SEQ ID NO: 18 - DNA, 42 bp, Artificial Sequence
  variant
ctagaggatgtaggactcccagctaaaactgccttctgccca

SEQ ID NO: 19 - DNA, 42 bp, Artificial Sequence
  variant
ctcgaggatgtaggactcccagctaaaactgccttctgccca

SEQ ID NO: 20 - DNA, 42 bp, Artificial Sequence
  variant
ctggaggatgtaggactcccagctaaaactgccttctgccca

SEQ ID NO: 21 - DNA, 42 bp, Artificial Sequence
  variant
cttaaggatgtaggactcccagctaaaactgccttctgccca

SEQ ID NO: 22 - DNA, 42 bp, Artificial Sequence
  variant
cttcaggatgtaggactcccagctaaaactgccttctgccca

SEQ ID NO: 23 - DNA, 42 bp, Artificial Sequence
  variant
ctttaggatgtaggactcccagctaaaactgccttctgccca

SEQ ID NO: 24 - DNA, 42 bp, Artificial Sequence
  variant
cttgcggatgtaggactcccagctaaaactgccttctgccca

SEQ ID NO: 25 - DNA, 42 bp, Artificial Sequence; identical to SEQ ID NO: 2
  variant

SEQ ID NO: 26 - DNA, 42 bp, Artificial Sequence; identical to SEQ ID NO: 2
  variant

SEQ ID NO: 27 - DNA, 42 bp, Artificial Sequence; identical to SEQ ID NO: 1
  Canonical reference sequence

SEQ ID NO: 28 - DNA, 44 bp, Artificial Sequence
  Variant
aaattgaggatgtaggactcccagctaaaactgccttctgccca

SEQ ID NO: 29 - DNA, 44 bp, Artificial Sequence
  Variant
atattgaggatgtaggactcccagctaaaactgccttctgccca

SEQ ID NO: 30 - DNA, 44 bp, Artificial Sequence
  Variant
agattgaggatgtaggactcccagctaaaactgccttctgccca

SEQ ID NO: 31 - DNA, 44 bp, Artificial Sequence
  Variant
acattgaggatgtaggactcccagctaaaactgccttctgccca

SEQ ID NO: 32 - DNA, 44 bp, Artificial Sequence
  Variant
taattgaggatgtaggactcccagctaaaactgccttctgccca

SEQ ID NO: 33 - DNA, 44 bp, Artificial Sequence
  Variant
ttattgaggatgtaggactcccagctaaaactgccttctgccca

SEQ ID NO: 34 - DNA, 44 bp, Artificial Sequence
  Variant
tgattgaggatgtaggactcccagctaaaactgccttctgccca

SEQ ID NO: 35 - DNA, 44 bp, Artificial Sequence
  Variant
tcattgaggatgtaggactcccagctaaaactgccttctgccca

SEQ ID NO: 36 - DNA, 44 bp, Artificial Sequence
  Variant
gaattgaggatgtaggactcccagctaaaactgccttctgccca

SEQ ID NO: 37 - DNA, 44 bp, Artificial Sequence
  Variant
gtattgaggatgtaggactcccagctaaaactgccttctgccca

SEQ ID NO: 38 - DNA, 44 bp, Artificial Sequence
  Variant
ggattgaggatgtaggactcccagctaaaactgccttctgccca

SEQ ID NO: 39 - DNA, 44 bp, Artificial Sequence
  Variant
gcattgaggatgtaggactcccagctaaaactgccttctgccca

SEQ ID NO: 40 - DNA, 44 bp, Artificial Sequence
  Variant
caattgaggatgtaggactcccagctaaaactgccttctgccca

SEQ ID NO: 41 - DNA, 44 bp, Artificial Sequence
  Variant
ctattgaggatgtaggactcccagctaaaactgccttctgccca

SEQ ID NO: 42 - DNA, 44 bp, Artificial Sequence
  Variant
cgattgaggatgtaggactcccagctaaaactgccttctgccca

SEQ ID NO: 43 - DNA, 44 bp, Artificial Sequence
  Variant
ccattgaggatgtaggactcccagctaaaactgccttctgccca

SEQ ID NO: 44 - DNA, 44 bp, Artificial Sequence; identical to SEQ ID NO: 28
  Variant

SEQ ID NO: 45 - DNA, 44 bp, Artificial Sequence
  Variant
aatttgaggatgtaggactcccagctaaaactgccttctgccca

SEQ ID NO: 46 - DNA, 44 bp, Artificial Sequence
  Variant
aagttgaggatgtaggactcccagctaaaactgccttctgccca

SEQ ID NO: 47 - DNA, 44 bp, Artificial Sequence
  Variant
aacttgaggatgtaggactcccagctaaaactgccttctgccca

SEQ ID NO: 48 - DNA, 44 bp, Artificial Sequence; identical to SEQ ID NO: 29
  Variant

SEQ ID NO: 49 - DNA, 44 bp, Artificial Sequence
  Variant
attttgaggatgtaggactcccagctaaaactgccttctgccca

SEQ ID NO: 50 - DNA, 44 bp, Artificial Sequence
  Variant
atgttgaggatgtaggactcccagctaaaactgccttctgccca

SEQ ID NO: 51 - DNA, 44 bp, Artificial Sequence
  Variant
atcttgaggatgtaggactcccagctaaaactgccttctgccca

SEQ ID NO: 52 - DNA, 44 bp, Artificial Sequence; identical to SEQ ID NO: 30
  Variant

SEQ ID NO: 53 - DNA, 44 bp, Artificial Sequence
  Variant
agtttgaggatgtaggactcccagctaaaactgccttctgccca

SEQ ID NO: 54 - DNA, 44 bp, Artificial Sequence
  Variant
aggttgaggatgtaggactcccagctaaaactgccttctgccca

SEQ ID NO: 55 - DNA, 44 bp, Artificial Sequence
  Variant
agcttgaggatgtaggactcccagctaaaactgccttctgccca

SEQ ID NO: 56 - DNA, 44 bp, Artificial Sequence; identical to SEQ ID NO: 31
  Variant

SEQ ID NO: 57 - DNA, 44 bp, Artificial Sequence
  Variant
actttgaggatgtaggactcccagctaaaactgccttctgccca

SEQ ID NO: 58 - DNA, 44 bp, Artificial Sequence
  Variant
acgttgaggatgtaggactcccagctaaaactgccttctgccca

SEQ ID NO: 59 - DNA, 44 bp, Artificial Sequence
  Variant
accttgaggatgtaggactcccagctaaaactgccttctgccca

SEQ ID NO: 60 - DNA, 44 bp, Artificial Sequence
  Variant
  misc_feature 3, 4: n = A,T,C or G
atnntgaggatgtaggactcccagctaaaactgccttctgccca

SEQ ID NO: 61 - DNA, 44 bp, Artificial Sequence
  Variant
  misc_feature 4, 5: n = A,T,C or G
attnngaggatgtaggactcccagctaaaactgccttctgccca

SEQ ID NO: 62 - DNA, 44 bp, Artificial Sequence
  Variant
  misc_feature 5, 6: n = A,T,C or G
attgnnaggatgtaggactcccagctaaaactgccttctgccca

SEQ ID NO: 63 - DNA, 44 bp, Artificial Sequence
  Variant
  misc_feature 6, 7: n = A,T,C or G
attgannggatgtaggactcccagctaaaactgccttctgccca

SEQ ID NO: 64 - DNA, 44 bp, Artificial Sequence
  Variant
  misc_feature 7, 8: n = A,T,C or G
attgagnngatgtaggactcccagctaaaactgccttctgccca

SEQ ID NO: 65 - DNA, 44 bp, Artificial Sequence
  Variant
  misc_feature 8, 9: n = A,T,C or G
attgaggnnatgtaggactcccagctaaaactgccttctgccca

SEQ ID NO: 66 - DNA, 44 bp, Artificial Sequence
  Variant
  misc_feature 9, 10: n = A,T,C or G
attgagganntgtaggactcccagctaaaactgccttctgccca

SEQ ID NO: 67 - DNA, 61 bp, Artificial Sequence
  Primer
tcgtcggcagcgtcagatgtgtataagagacaggcaccatctcacaattgccagttaacgt

SEQ ID NO: 68 - DNA, 60 bp, Artificial Sequence
  Primer
gtctcgtgggctcggagatgtgtataagagacagacacagcaaagcagaaactcacatcg

SEQ ID NO: 69 - DNA, 59 bp, Artificial Sequence
  Primer
tcgtcggcagcgtcagatgtgtataagagacagtgccagttaacgtcttccttctctct

SEQ ID NO: 70 - DNA, 56 bp, Artificial Sequence
  Primer
gtctcgtgggctcggagatgtgtataagagacagtgaggttcagagccatggaccc

SEQ ID NO: 71 - DNA, 56 bp, Artificial Sequence
  Primer
tcgtcggcagcgtcagatgtgtataagagacagcattcatgcgtcttcacctggaa

SEQ ID NO: 72 - DNA, 54 bp, Artificial Sequence
  Primer
gtctcgtgggctcggagatgtgtataagagacaggtgatgagctgcacggtgga

SEQ ID NO: 73 - DNA, 56 bp, Artificial Sequence; identical to SEQ ID NO: 71
  Primer

SEQ ID NO: 74 - DNA, 54 bp, Artificial Sequence; identical to SEQ ID NO: 72
  Primer

SEQ ID NO: 75 - DNA, 55 bp, Artificial Sequence
  Primer
tcgtcggcagcgtcagatgtgtataagagacagagcatggtgagggctgaggtga

SEQ ID NO: 76 - DNA, 59 bp, Artificial Sequence
  Primer
gtctcgtgggctcggagatgtgtataagagacagccttaccttatacaccgtgccgaac

SEQ ID NO: 77 - DNA, 58 bp, Artificial Sequence
  Primer
tcgtcggcagcgtcagatgtgtataagagacagtgaaaacaccgcagcatgtcaagat

SEQ ID NO: 78 - DNA, 60 bp, Artificial Sequence
  Primer
gtctcgtgggctcggagatgtgtataagagacagacaatacagctagtgggaaggcagcc

SEQ ID NO: 79 - DNA, 54 bp, Artificial Sequence
  Primer
tcgtcggcagcgtcagatgtgtataagagacagggcctgctgaaaatgactgaa

SEQ ID NO: 80 - DNA, 59 bp, Artificial Sequence
  Primer
gtctcgtgggctcggagatgtgtataagagacagtcaaagaatggtcctgcaccagtaa

SEQ ID NO: 81 - DNA, 55 bp, Artificial Sequence
  Primer
tcgtcggcagcgtcagatgtgtataagagacagtccagactgtgtttctcccttc

SEQ ID NO: 82 - DNA, 56 bp, Artificial Sequence
  Primer
gtctcgtgggctcggagatgtgtataagagacagagaaagccctccccagtcctca

SEQ ID NO: 83 - DNA, 53 bp, Artificial Sequence
  Primer
tcgtcggcagcgtcagatgtgtataagagacaggggccatggctgtggtttgt

SEQ ID NO: 84 - DNA, 54 bp, Artificial Sequence
  Primer
gtctcgtgggctcggagatgtgtataagagacagcagctgcaccgtggatgtca

SEQ ID NO: 85 - DNA, 55 bp, Artificial Sequence
  Primer
tcgtcggcagcgtcagatgtgtataagagacagaccaggaccagaggaaacctca

SEQ ID NO: 86 - DNA, 59 bp, Artificial Sequence
  Primer
gtctcgtgggctcggagatgtgtataagagacagtggagaaaagtatcggttggctttg

SEQ ID NO: 87 - DNA, 58 bp, Artificial Sequence
  Primer
tcgtcggcagcgtcagatgtgtataagagacagccttttgaagaccataacccaccac

SEQ ID NO: 88 - DNA, 58 bp, Artificial Sequence
  Primer
gtctcgtgggctcggagatgtgtataagagacaggcctttaaaaatttgccccgatgt

SEQ ID NO: 89 - DNA, 59 bp, Artificial Sequence
  Primer
tcgtcggcagcgtcagatgtgtataagagacagcaccagggagtaactattcccagtca

SEQ ID NO: 90 - DNA, 55 bp, Artificial Sequence
  Primer
gtctcgtgggctcggagatgtgtataagagacagtgcaagttccgccactgaaca

SEQ ID NO: 91 - DNA, 58 bp, Artificial Sequence
  Primer
tcgtcggcagcgtcagatgtgtataagagacagcagtttgacagttaaaggcatttcc

SEQ ID NO: 92 - DNA, 56 bp, Artificial Sequence
  Primer
gtctcgtgggctcggagatgtgtataagagacagcacaggtaacggctgagggaac

SEQ ID NO: 93 - DNA, 60 bp, Artificial Sequence
  Primer
tcgtcggcagcgtcagatgtgtataagagacagctcttcataatgcttgctctgatagga

SEQ ID NO: 94 - DNA, 54 bp, Artificial Sequence
  Primer
gtctcgtgggctcggagatgtgtataagagacagtgatgggacccactccatcg

SEQ ID NO: 95 - DNA, 59 bp, Artificial Sequence
  Primer
tcgtcggcagcgtcagatgtgtataagagacagccttcctcttcctacagtactcccct

SEQ ID NO: 96 - DNA, 55 bp, Artificial Sequence
  Primer
gtctcgtgggctcggagatgtgtataagagacagcacaacctccgtcatgtgctg

SEQ ID NO: 97 - DNA, 56 bp, Artificial Sequence
  Primer
tcgtcggcagcgtcagatgtgtataagagacagccctgtgcagctgtgggttgatt

SEQ ID NO: 98 - DNA, 54 bp, Artificial Sequence
  Primer
gtctcgtgggctcggagatgtgtataagagacagcaaccagccctgtcgtctct

SEQ ID NO: 99 - DNA, 59 bp, Artificial Sequence
  Primer
tcgtcggcagcgtcagatgtgtataagagacagcctcactgattgctcttaggtctggc

SEQ ID NO: 100 - DNA, 59 bp, Artificial Sequence
  Primer
gtctcgtgggctcggagatgtgtataagagacagcagagaccccagttgcaaaccagac

SEQ ID NO: 101 - DNA, 57 bp, Artificial Sequence
  Primer
tcgtcggcagcgtcagatgtgtataagagacagggctctgactgtaccaccatccac

SEQ ID NO: 102 - DNA, 58 bp, Artificial Sequence
  Primer
gtctcgtgggctcggagatgtgtataagagacaggaagaaatcggtaagaggtgggcc

SEQ ID NO: 103 - DNA, 53 bp, Artificial Sequence
  Primer
tcgtcggcagcgtcagatgtgtataagagacagtgggacggaacagctttgag

SEQ ID NO: 104 - DNA, 58 bp, Artificial Sequence
  Primer
gtctcgtgggctcggagatgtgtataagagacagaccgcttcttgtcctgcttgctta

SEQ ID NO: 105 - DNA, 58 bp, Artificial Sequence
  Primer
tcgtcggcagcgtcagatgtgtataagagacagattgtgaagatctgtgactttggcc

SEQ ID NO: 106 - DNA, 59 bp, Artificial Sequence
  Primer
gtctcgtgggctcggagatgtgtataagagacagaggcaccgaatctctagaagcaaca

SEQ ID NO: 107 - DNA, 60 bp, Artificial Sequence
  Primer
tcgtcggcagcgtcagatgtgtataagagacagactagctagagacaatgaattaaggga

SEQ ID NO: 108 - DNA, 59 bp, Artificial Sequence
  Primer
gtctcgtgggctcggagatgtgtataagagacagagaatctccattttagcacttacct

SEQ ID NO: 109 - DNA, 57 bp, Artificial Sequence
  Primer
tcgtcggcagcgtcagatgtgtataagagacagaatgatgcttggctctggaatgcc

SEQ ID NO: 110 - DNA, 59 bp, Artificial Sequence
  Primer
gtctcgtgggctcggagatgtgtataagagacagtgcatgctgtttaattgtgtggaag

SEQ ID NO: 111 - DNA, 59 bp, Artificial Sequence
  Primer
tcgtcggcagcgtcagatgtgtataagagacagcaatggcacggttgaatgtaaggctt

SEQ ID NO: 112 - DNA, 61 bp, Artificial Sequence
  Primer
gtctcgtgggctcggagatgtgtataagagacagtgactgatatggtagacagagcctaaa

SEQ ID NO: 113 - DNA, 58 bp, Artificial Sequence
  Primer
tcgtcggcagcgtcagatgtgtataagagacagtctccccacagaaacccatgtatga

SEQ ID NO: 114 - DNA, 59 bp, Artificial Sequence
  Primer
gtctcgtgggctcggagatgtgtataagagacagggaaagcccctgtttcatactgacc

SEQ ID NO: 115 - DNA, 61 bp, Artificial Sequence
  Primer
tcgtcggcagcgtcagatgtgtataagagacagtttcttttctcctccaacctaatagtgt

SEQ ID NO: 116 - DNA, 59 bp, Artificial Sequence
  Primer
gtctcgtgggctcggagatgtgtataagagacagatgggtactcacgtttcctttaacc

SEQ ID NO: 117 - DNA, 60 bp, Artificial Sequence
  Primer
tcgtcggcagcgtcagatgtgtataagagacagtgatgtctatgaagtgttgtggttcct

SEQ ID NO: 118 - DNA, 59 bp, Artificial Sequence
  Primer
gtctcgtgggctcggagatgtgtataagagacagcaaacacggtagggaaagttctcac

SEQ ID NO: 119 - DNA, 59 bp, Artificial Sequence
  Primer
tcgtcggcagcgtcagatgtgtataagagacagaactgcctattcctaactgactcatc

SEQ ID NO: 120 - DNA, 55 bp, Artificial Sequence
  Primer
gtctcgtgggctcggagatgtgtataagagacaggctgcagaaacatttggcaca

SEQ ID NO: 121 - DNA, 60 bp, Artificial Sequence
  Primer
tcgtcggcagcgtcagatgtgtataagagacagtgcacgtactcaccatttgtctttgca

SEQ ID NO: 122 - DNA, 57 bp, Artificial Sequence
  Primer
gtctcgtgggctcggagatgtgtataagagacagtgtgcatctttgttgctgtcctt

SEQ ID NO: 123 - DNA, 60 bp, Artificial Sequence
  Primer
tcgtcggcagcgtcagatgtgtataagagacagttgcactccaggataatacacatcaca

SEQ ID NO: 124 - DNA, 56 bp, Artificial Sequence
  Primer
gtctcgtgggctcggagatgtgtataagagacagcagcctcacattgcccctgaca

SEQ ID NO: 125 - DNA, 55 bp, Artificial Sequence
  Primer
tcgtcggcagcgtcagatgtgtataagagacagggctttctccggcatcatgatt

SEQ ID NO: 126 - DNA, 56 bp, Artificial Sequence
  Primer
gtctcgtgggctcggagatgtgtataagagacagtgaggtgcagaatcaggggctc

SEQ ID NO: 127 - DNA, 55 bp, Artificial Sequence
  Primer
tcgtcggcagcgtcagatgtgtataagagacagacctcagccccgtgtgtatcct

SEQ ID NO: 128 - DNA, 56 bp, Artificial Sequence
  Primer
gtctcgtgggctcggagatgtgtataagagacagtcagctcaccttggctcacagg

SEQ ID NO: 129 - DNA, 55 bp, Artificial Sequence
  Primer
tcgtcggcagcgtcagatgtgtataagagacagcctgtgagccaaggtgagctga

SEQ ID NO: 130 - DNA, 59 bp, Artificial Sequence
  Primer
gtctcgtgggctcggagatgtgtataagagacagagctcctattatcctgtccctttga

SEQ ID NO: 131 - DNA, 60 bp, Artificial Sequence
  Primer
tcgtcggcagcgtcagatgtgtataagagacagcctcaaagggacaggataataggagct

SEQ ID NO: 132 - DNA, 59 bp, Artificial Sequence
  Primer
gtctcgtgggctcggagatgtgtataagagacagaggatgttctggaaggcaaactcca

SEQ ID NO: 133 - DNA, 58 bp, Artificial Sequence
  Primer
tcgtcggcagcgtcagatgtgtataagagacagtttgccttccagaacatcctcacat

SEQ ID NO: 134 - DNA, 59 bp, Artificial Sequence
  Primer
gtctcgtgggctcggagatgtgtataagagacagctggggatctgtgctctaattccgc

SEQ ID NO: 135 - DNA, 58 bp, Artificial Sequence
  Primer
tcgtcggcagcgtcagatgtgtataagagacaggaggcggaattagagcacagatccc

SEQ ID NO: 136 - DNA, 59 bp, Artificial Sequence
  Primer
gtctcgtgggctcggagatgtgtataagagacagcctaaggaagtttcagcaaggccct

SEQ ID NO: 137 - DNA, 58 bp, Artificial Sequence
  Primer
tcgtcggcagcgtcagatgtgtataagagacagcctctgatgactgactttggctcca

SEQ ID NO: 138 - DNA, 59 bp, Artificial Sequence
  Primer
gtctcgtgggctcggagatgtgtataagagacagagagtcatgttagtctggttcctcc

SEQ ID NO: 139 - DNA, 59 bp, Artificial Sequence
  Primer
tcgtcggcagcgtcagatgtgtataagagacagggaaccagactaacatgactctgccc

SEQ ID NO: 140 - DNA, 56 bp, Artificial Sequence
  Primer
gtctcgtgggctcggagatgtgtataagagacagtggtcagctgcaacatggcctg

SEQ ID NO: 141 - DNA, 58 bp, Artificial Sequence
  Primer
tcgtcggcagcgtcagatgtgtataagagacagaaaactactgtagagcccacacctg

SEQ ID NO: 142 - DNA, 64 bp, Artificial Sequence
  Primer
gtctcgtgggctcggagatgtgtataagagacaggggaatggttcagtatagtcaaatgtgggt

SEQ ID NO: 143 - DNA, 60 bp, Artificial Sequence
  Primer
tcgtcggcagcgtcagatgtgtataagagacagtgactatactgaaccattccctttagg

SEQ ID NO: 144 - DNA, 58 bp, Artificial Sequence
  Primer
gtctcgtgggctcggagatgtgtataagagacagttgaggtaaagctgaatggatgcc

SEQ ID NO: 145 - DNA, 60 bp, Artificial Sequence
  Primer
tcgtcggcagcgtcagatgtgtataagagacaggctggttctgggatatctgttagagca

SEQ ID NO: 146 - DNA, 56 bp, Artificial Sequence
  Primer
gtctcgtgggctcggagatgtgtataagagacagccgtagaaaagggcaaagagga

SEQ ID NO: 147 - DNA, 59 bp, Artificial Sequence
  Primer
tcgtcggcagcgtcagatgtgtataagagacagtcctctttgcccttttctacggtaaa

SEQ ID NO: 148 - DNA, 61 bp, Artificial Sequence
  Primer
gtctcgtgggctcggagatgtgtataagagacagtggtgaattattgaggactggctgacc

SEQ ID NO: 149 - DNA, 58 bp, Artificial Sequence
  Primer
tcgtcggcagcgtcagatgtgtataagagacagcagccagtcctcaataattcaccaa

SEQ ID NO: 150 - DNA, 59 bp, Artificial Sequence
  Primer
gtctcgtgggctcggagatgtgtataagagacagttctgagcacacgaacagggattcc

SEQ ID NO: 151 - DNA, 58 bp, Artificial Sequence
  Primer
tcgtcggcagcgtcagatgtgtataagagacagtcgtgtgctcagaaagacccgattt

SEQ ID NO: 152 - DNA, 60 bp, Artificial Sequence
  Primer
gtctcgtgggctcggagatgtgtataagagacagagtcaaggagcatctgaatatctgtc

SEQ ID NO: 153 - DNA, 58 bp, Artificial Sequence
  Primer
tcgtcggcagcgtcagatgtgtataagagacagtgctccttgacttctggatggcatt

SEQ ID NO: 154 - DNA, 59 bp, Artificial Sequence
  Primer
gtctcgtgggctcggagatgtgtataagagacagctgccattcttctcgtgtgtaaatt

SEQ ID NO: 155 - DNA, 58 bp, Artificial Sequence
  Primer
tcgtcggcagcgtcagatgtgtataagagacaggaatggcagtgggttgagggttctt

SEQ ID NO: 156 - DNA, 59 bp, Artificial Sequence
  Primer
gtctcgtgggctcggagatgtgtataagagacagcttcctgtccccaacctgaacacaa

SEQ ID NO: 157 - DNA, 61 bp, Artificial Sequence
  Primer
tcgtcggcagcgtcagatgtgtataagagacagagccttaatatttgtgttcaggttgggg

SEQ ID NO: 158 - DNA, 60 bp, Artificial Sequence
  Primer
gtctcgtgggctcggagatgtgtataagagacagtgtggacacttactctggtctggact

SEQ ID NO: 159 - DNA, 55 bp, Artificial Sequence
  Primer
tcgtcggcagcgtcagatgtgtataagagacaggcctgtgttgtactttgccaca

SEQ ID NO: 160 - DNA, 61 bp, Artificial Sequence
  Primer
gtctcgtgggctcggagatgtgtataagagacaggtcacagtacactcaaattttggttgg

SEQ ID NO: 161 - DNA, 60 bp, Artificial Sequence
  Primer
tcgtcggcagcgtcagatgtgtataagagacagggcagtcttaccaaccaaaatttgagt

SEQ ID NO: 162 - DNA, 65 bp, Artificial Sequence
  Primer
gtctcgtgggctcggagatgtgtataagagacagacaaatacctcatacctacttaagaaacaga

SEQ ID NO: 163 - DNA, 61 bp, Artificial Sequence
  Primer
tcgtcggcagcgtcagatgtgtataagagacagttccaaaccatttcttccttaaacatga

SEQ ID NO: 164 - DNA, 60 bp, Artificial Sequence
  Primer
gtctcgtgggctcggagatgtgtataagagacagtcctttgcaagacttaagaatggtga

SEQ ID NO: 165 - DNA, 59 bp, Artificial Sequence
  Primer
tcgtcggcagcgtcagatgtgtataagagacagtgggtaagtggaagttgagagtatct

SEQ ID NO: 166 - DNA, 61 bp, Artificial Sequence
  Primer
gtctcgtgggctcggagatgtgtataagagacagtccaaacttaatcacaaacctcaccct

SEQ ID NO: 167 - DNA, 58 bp, Artificial Sequence
  Primer
tcgtcggcagcgtcagatgtgtataagagacagtatttggcaggcagtgtaaacttgc

SEQ ID NO: 168 - DNA, 60 bp, Artificial Sequence
  Primer
gtctcgtgggctcggagatgtgtataagagacaggtctttcttatgggcctgtatttctg

SEQ ID NO: 169 - DNA, 58 bp, Artificial Sequence
  Primer
tcgtcggcagcgtcagatgtgtataagagacagtgaaatatcagaaatacaggcccat

SEQ ID NO: 170 - DNA, 59 bp, Artificial Sequence
  Primer
gtctcgtgggctcggagatgtgtataagagacagaacacttaaaatcctcccagaatga

SEQ ID NO: 171 - DNA, 61 bp, Artificial Sequence
  Primer
tcgtcggcagcgtcagatgtgtataagagacagcatcattctgggaggattttaagtgttt

SEQ ID NO: 172 - DNA, 62 bp, Artificial Sequence
  Primer
gtctcgtgggctcggagatgtgtataagagacagtctgagagtactactggctttatttgga

SEQ ID NO: 173 - DNA, 64 bp, Artificial Sequence
  Primer
tcgtcggcagcgtcagatgtgtataagagacaggcacagggaaataagcctagaatttgctttt

SEQ ID NO: 174 - DNA, 59 bp, `Artificial Sequence
  Primer
gtctcgtgggctcggagatgtgtataagagacaggggcttgcttgatttggaggagaac

SEQ ID NO: 175 - DNA, 58 bp, Artificial Sequence
  Primer
tcgtcggcagcgtcagatgtgtataagagacaggcaagttctcctccaaatcaagcaa

SEQ ID NO: 176 - DNA, 56 bp, Artificial Sequence
  Primer
gtctcgtgggctcggagatgtgtataagagacagtgtccttaggtcagatagtggt

SEQ ID NO: 177 - DNA, 55 bp, Artificial Sequence
  Primer
tcgtcggcagcgtcagatgtgtataagagacagtggagtcatacaatgtgtggtc

SEQ ID NO: 178 - DNA, 56 bp, Artificial Sequence
  Primer
gtctcgtgggctcggagatgtgtataagagacagggtcagatgtttgaaaccaacc

SEQ ID NO: 179 - DNA, 54 bp, Artificial Sequence
  Primer
tcgtcggcagcgtcagatgtgtataagagacaggccacagtgcccagccttcac

SEQ ID NO: 180 - DNA, 59 bp, Artificial Sequence
  Primer
gtctcgtgggctcggagatgtgtataagagacagaccaggttcaaaatgggaaggtaga

SEQ ID NO: 181 - DNA, 58 bp, Artificial Sequence
  Primer
tcgtcggcagcgtcagatgtgtataagagacagtctaccttcccattttgaacctggt

SEQ ID NO: 182 - DNA, 62 bp, Artificial Sequence
  Primer
gtctcgtgggctcggagatgtgtataagagacagagcccagttttcttgtatacccatagca

SEQ ID NO: 183 - DNA, 58 bp, Artificial Sequence
  Primer
tcgtcggcagcgtcagatgtgtataagagacagttctctgctagtaggtcaaaagcca

SEQ ID NO: 184 - DNA, 57 bp, Artificial Sequence
  Primer
gtctcgtgggctcggagatgtgtataagagacagcaaagctgtgactaggctcaagt

SEQ ID NO: 185 - DNA, 60 bp, Artificial Sequence
  Primer
tcgtcggcagcgtcagatgtgtataagagacagagctaactatgtggtatcccctaagct

SEQ ID NO: 186 - DNA, 60 bp, Artificial Sequence
  Primer
gtctcgtgggctcggagatgtgtataagagacaggcaaagcaggtagtaaagtttagggt

SEQ ID NO: 187 - DNA, 62 bp, Artificial Sequence
  Primer
tcgtcggcagcgtcagatgtgtataagagacagagttcatagtaatcaaagaaaagtgcgtt

SEQ ID NO: 188 - DNA, 60 bp, Artificial Sequence
  Primer
gtctcgtgggctcggagatgtgtataagagacagtggattcatctagctcaaatcactgt

SEQ ID NO: 189 - DNA, 58 bp, Artificial Sequence
  Primer
tcgtcggcagcgtcagatgtgtataagagacagacctgtctgttgtccccacctactt

SEQ ID NO: 190 - DNA, 59 bp, Artificial Sequence
  Primer
gtctcgtgggctcggagatgtgtataagagacagatgccactgatcaaccgcaactctt

SEQ ID NO: 191 - DNA, 58 bp, Artificial Sequence
  Primer
tcgtcggcagcgtcagatgtgtataagagacaggtgttgtgtgcaggccaaaggtatg

SEQ ID NO: 192 - DNA, 59 bp, Artificial Sequence
  Primer
gtctcgtgggctcggagatgtgtataagagacagaataccccataccccatctcagcga

SEQ ID NO: 193 - DNA, 58 bp, Artificial Sequence
  Primer
tcgtcggcagcgtcagatgtgtataagagacagcaggaaaggaaggacagttgcctcc

SEQ ID NO: 194 - DNA, 59 bp, Artificial Sequence
  Primer
gtctcgtgggctcggagatgtgtataagagacaggcaaggctctgaacaaaagcacctg

SEQ ID NO: 195 - DNA, 58 bp, Artificial Sequence
  Primer
tcgtcggcagcgtcagatgtgtataagagacaggccagtcttgcagttaacaaagcgt

SEQ ID NO: 196 - DNA, 59 bp, Artificial Sequence
  Primer
gtctcgtgggctcggagatgtgtataagagacaggaacaggcacagctcagaacaccat

SEQ ID NO: 197 - DNA, 58 bp, Artificial Sequence
  Primer
tcgtcggcagcgtcagatgtgtataagagacagtctcccttctccactctgcctgaat

SEQ ID NO: 198 - DNA, 59 bp, Artificial Sequence
  Primer
gtctcgtgggctcggagatgtgtataagagacagatgcttgcccttcagtttccttggg

SEQ ID NO: 199 - DNA, 58 bp, Artificial Sequence
  Primer
tcgtcggcagcgtcagatgtgtataagagacaggatgtacgcagggcaatctctgagg

SEQ ID NO: 200 - DNA, 58 bp, Artificial Sequence
  Primer
gtctcgtgggctcggagatgtgtataagagacagtggagcacaacccagcagaactag

SEQ ID NO: 201 - DNA, 58 bp, Artificial Sequence
  Primer
tcgtcggcagcgtcagatgtgtataagagacagaggcccttcaagtcctttagaatct

SEQ ID NO: 202 - DNA, 59 bp, Artificial Sequence
  Primer
gtctcgtgggctcggagatgtgtataagagacagagattctctggatcctgtgctaatg

SEQ ID NO: 203 - DNA, 100 bp, Homo sapiens
tctcctttttcttttcctcactgcagcccttcttcccaaccttagtgtaggggtccttggcatccattcagctttacctcaaatcagttttcctaaaaaa

SEQ ID NO: 204 - DNA, 100 bp, Homo sapiens
aaaacggaagctcccaaccttccccccaccagagcagctgcagttccctgaggagcccctgattctgcacctcagccccgtgtgtatcctcctggctgat

SEQ ID NO: 205 - DNA, 100 bp, Artificial Sequence
  Rearranged sequence
taaacggaagctcccaaccttccccccaccagagcagctgcagttccctgaggagcccctgattctgcacctcagccccgtgtgtatcctcctggctgat

SEQ ID NO: 206 - DNA, 100 bp, Artificial Sequence
  Rearranged sequence
tcaacggaagctcccaaccttccccccaccagagcagctgcagttccctgaggagcccctgattctgcacctcagccccgtgtgtatcctcctggctgat

SEQ ID NO: 207 - DNA, 100 bp, Artificial Sequence
  Rearranged sequence
tctacggaagctcccaaccttccccccaccagagcagctgcagttccctgaggagcccctgattctgcacctcagccccgtgtgtatcctcctggctgat

SEQ ID NO: 208 - DNA, 100 bp, Artificial Sequence
  Rearranged sequence
tctccggaagctcccaaccttccccccaccagagcagctgcagttccctgaggagcccctgattctgcacctcagccccgtgtgtatcctcctggctgat

SEQ ID NO: 209 - DNA, 100 bp, Artificial Sequence; identical to SEQ ID NO: 208
  Rearranged sequence

SEQ ID NO: 210 - DNA, 100 bp, Artificial Sequence
  Rearranged sequence
tctcctgaagctcccaaccttccccccaccagagcagctgcagttccctgaggagcccctgattctgcacctcagccccgtgtgtatcctcctggctgat

SEQ ID NO: 211 - DNA, 100 bp, Artificial Sequence
  Rearranged sequence
tctccttaagctcccaaccttccccccaccagagcagctgcagttccctgaggagcccctgattctgcacctcagccccgtgtgtatcctcctggctgat

SEQ ID NO: 212 - DNA, 100 bp, Artificial Sequence
  Rearranged sequence
tctcctttagctcccaaccttccccccaccagagcagctgcagttccctgaggagcccctgattctgcacctcagccccgtgtgtatcctcctggctgat

SEQ ID NO: 213 - DNA, 100 bp, Artificial Sequence
  Rearranged sequence
tctccttttgctcccaaccttccccccaccagagcagctgcagttccctgaggagcccctgattctgcacctcagccccgtgtgtatcctcctggctgat

SEQ ID NO: 214 - DNA, 99 bp, Artificial Sequence
  Rearranged sequence
tctcctttttcttttcctcactgcagcccttcttcccaaccttagtgtaaggagcccctgattctgcacctcagccccgtgtgtatcctcctggctgat

SEQ ID NO: 215 - DNA, 100 bp, Artificial Sequence
  Rearranged sequence
tctcctttttcttttcctcactgcagcccttcttcccaaccttagtgtaggggtccttggcatccattcagctttacctcaaatcagtttcctggctgat

SEQ ID NO: 216 - DNA, 100 bp, Artificial Sequence
  Rearranged sequence
tctcctttttcttttcctcactgcagcccttcttcccaaccttagtgtaggggtccttggcatccattcagctttacctcaaatcagttttctggctgat

SEQ ID NO: 217 - DNA, 100 bp, Artificial Sequence; identical to SEQ ID NO: 216
  Rearranged sequence

SEQ ID NO: 218 - DNA, 100 bp, Artificial Sequence
  Rearranged sequence
tctcctttttcttttcctcactgcagcccttcttcccaaccttagtgtaggggtccttggcatccattcagctttacctcaaatcagttttccggctgat

SEQ ID NO: 219 - DNA, 100 bp, Artificial Sequence
  Rearranged sequence
tctcctttttcttttcctcactgcagcccttcttcccaaccttagtgtaggggtccttggcatccattcagctttacctcaaatcagttttcctgctgat

SEQ ID NO: 220 - DNA, 100 bp, Artificial Sequence
  Rearranged sequence
tctcctttttcttttcctcactgcagcccttcttcccaaccttagtgtaggggtccttggcatccattcagctttacctcaaatcagttttcctactgat

SEQ ID NO: 221 - DNA, 100 bp, Artificial Sequence
  Rearranged sequence
tctcctttttcttttcctcactgcagcccttcttcccaaccttagtgtaggggtccttggcatccattcagctttacctcaaatcagttttcctaatgat

SEQ ID NO: 222 - DNA, 100 bp, Artificial Sequence
  Rearranged sequence
tctcctttttcttttcctcactgcagcccttcttcccaaccttagtgtaggggtccttggcatccattcagctttacctcaaatcagttttcctaaagat

SEQ ID NO: 223 - DNA, 100 bp, Artificial Sequence
  Rearranged sequence
tctcctttttcttttcctcactgcagcccttcttcccaaccttagtgtaggggtccttggcatccattcagctttacctcaaatcagttttcctaaaaat

SEQ ID NO: 224 - DNA, 100 bp, Artificial Sequence; identical to SEQ ID NO: 223
  Rearranged sequence

SEQ ID NO: 225 - DNA, 28 bp, Artificial Sequence
  Primer
gcaccatctcacaattgccagttaacgt

SEQ ID NO: 226 - DNA, 26 bp, Artificial Sequence
  Primer
acacagcaaagcagaaactcacatcg

SEQ ID NO: 227 - DNA, 26 bp, Artificial Sequence
  Primer
tgccagttaacgtcttccttctctct

SEQ ID NO: 228 - DNA, 22 bp, Artificial Sequence
  Primer
tgaggttcagagccatggaccc

SEQ ID NO: 229 - DNA, 23 bp, Artificial Sequence
  Primer
cattcatgcgtcttcacctggaa

SEQ ID NO: 230 - DNA, 20 bp, Artificial Sequence
  Primer
gtgatgagctgcacggtgga

SEQ ID NO: 231 - DNA, 22 bp, Artificial Sequence
  Primer
agcatggtgagggctgaggtga

SEQ ID NO: 232 - DNA, 25 bp, Artificial Sequence
  Primer
ccttaccttatacaccgtgccgaac

SEQ ID NO: 233 - DNA, 20 bp, Artificial Sequence
  Primer
atctgcctcacctccaccgt

SEQ ID NO: 234 - DNA, 25 bp, Artificial Sequence
  Primer
tggctccttatctcccctccccgta

SEQ ID NO: 235 - DNA, 25 bp, Artificial Sequence
  Primer
tgaaaacaccgcagcatgtcaagat

SEQ ID NO: 236 - DNA, 26 bp, Artificial Sequence
  Primer
acaatacagctagtgggaaggcagcc

SEQ ID NO: 237 - DNA, 21 bp, Artificial Sequence
  Primer
ggcctgctgaaaatgactgaa

SEQ ID NO: 238 - DNA, 25 bp, Artificial Sequence
  Primer
tcaaagaatggtcctgcaccagtaa

SEQ ID NO: 239 - DNA, 22 bp, Artificial Sequence
  Primer
tccagactgtgtttctcccttc

SEQ ID NO: 240 - DNA, 22 bp, Artificial Sequence
  Primer
agaaagccctccccagtcctca

SEQ ID NO: 241 - DNA, 20 bp, Artificial Sequence
  Primer
gggccatggctgtggtttgt

SEQ ID NO: 242 - DNA, 20 bp, Artificial Sequence
  Primer
cagctgcaccgtggatgtca

SEQ ID NO: 243 - DNA, 22 bp, Artificial Sequence
  Primer
accaggaccagaggaaacctca

SEQ ID NO: 244 - DNA, 25 bp, Artificial Sequence
  Primer
tggagaaaagtatcggttggctttg

SEQ ID NO: 245 - DNA, 25 bp, Artificial Sequence
  Primer
ccttttgaagaccataacccaccac

SEQ ID NO: 246 - DNA, 24 bp, Artificial Sequence
  Primer
gcctttaaaaatttgccccgatgt

SEQ ID NO: 247 - DNA, 26 bp, Artificial Sequence
  Primer
caccagggagtaactattcccagtca

SEQ ID NO: 248 - DNA, 21 bp, Artificial Sequence
  Primer
tgcaagttccgccactgaaca

SEQ ID NO: 249 - DNA, 25 bp, Artificial Sequence
  Primer
cagtttgacagttaaaggcatttcc

SEQ ID NO: 250 - DNA, 22 bp, Artificial Sequence
  Primer
cacaggtaacggctgagggaac

SEQ ID NO: 251 - DNA, 27 bp, Artificial Sequence
  Primer
ctcttcataatgcttgctctgatagga

SEQ ID NO: 252 - DNA, 20 bp, Artificial Sequence
  Primer
tgatgggacccactccatcg

SEQ ID NO: 253 - DNA, 26 bp, Artificial Sequence
  Primer
ccttcctcttcctacagtactcccct

SEQ ID NO: 254 - DNA, 21 bp, Artificial Sequence
  Primer
cacaacctccgtcatgtgctg

SEQ ID NO: 255 - DNA, 23 bp, Artificial Sequence
  Primer
ccctgtgcagctgtgggttgatt

SEQ ID NO: 256 - DNA, 20 bp, Artificial Sequence
  Primer
caaccagccctgtcgtctct

SEQ ID NO: 257 - DNA, 26 bp, Artificial Sequence
  Primer
cctcactgattgctcttaggtctggc

SEQ ID NO: 258 - DNA, 25 bp, Artificial Sequence
  Primer
cagagaccccagttgcaaaccagac

SEQ ID NO: 259 - DNA, 24 bp, Artificial Sequence
  Primer
ggctctgactgtaccaccatccac

SEQ ID NO: 260 - DNA, 24 bp, Artificial Sequence
  Primer
gaagaaatcggtaagaggtgggcc

SEQ ID NO: 261 - DNA, 20 bp, Artificial Sequence
  Primer
tgggacggaacagctttgag

SEQ ID NO: 262 - DNA, 24 bp, Artificial Sequence
  Primer
accgcttcttgtcctgcttgctta

SEQ ID NO: 263 - DNA, 25 bp, Artificial Sequence
  Primer
attgtgaagatctgtgactttggcc

SEQ ID NO: 264 - DNA, 25 bp, Artificial Sequence
  Primer
aggcaccgaatctctagaagcaaca

SEQ ID NO: 265 - DNA, 27 bp, Artificial Sequence
  Primer
actagctagagacaatgaattaaggga

SEQ ID NO: 266 - DNA, 25 bp, Artificial Sequence
  Primer
agaatctccattttagcacttacct

SEQ ID NO: 267 - DNA, 24 bp, Artificial Sequence
  Primer
aatgatgcttggctctggaatgcc

SEQ ID NO: 268 - DNA, 25 bp, Artificial Sequence
  Primer
tgcatgctgtttaattgtgtggaag

SEQ ID NO: 269 - DNA, 26 bp, Artificial Sequence
  Primer
caatggcacggttgaatgtaaggctt

SEQ ID NO: 270 - DNA, 27 bp, Artificial Sequence
  Primer
tgactgatatggtagacagagcctaaa

SEQ ID NO: 271 - DNA, 25 bp, Artificial Sequence
  Primer
tctccccacagaaacccatgtatga

SEQ ID NO: 272 - DNA, 25 bp, Artificial Sequence
  Primer
ggaaagcccctgtttcatactgacc

SEQ ID NO: 273 - DNA, 28 bp, Artificial Sequence
  Primer
tttcttttctcctccaacctaatagtgt

SEQ ID NO: 274 - DNA, 25 bp, Artificial Sequence
  Primer
atgggtactcacgtttcctttaacc

SEQ ID NO: 275 - DNA, 27 bp, Artificial Sequence
  Primer
tgatgtctatgaagtgttgtggttcct

SEQ ID NO: 276 - DNA, 25 bp, Artificial Sequence
  Primer
caaacacggtagggaaagttctcac

SEQ ID NO: 277 - DNA, 26 bp, Artificial Sequence
  Primer
aactgcctattcctaactgactcatc

SEQ ID NO: 278 - DNA, 21 bp, Artificial Sequence
  Primer
gctgcagaaacatttggcaca

SEQ ID NO: 279 - DNA, 27 bp, Artificial Sequence
  Primer
tgcacgtactcaccatttgtctttgca

SEQ ID NO: 280 - DNA, 23 bp, Artificial Sequence
  Primer
tgtgcatctttgttgctgtcctt

SEQ ID NO: 281 - DNA, 27 bp, Artificial Sequence
  Primer
ttgcactccaggataatacacatcaca

SEQ ID NO: 282 - DNA, 22 bp, Artificial Sequence
  Primer
cagcctcacattgcccctgaca

SEQ ID NO: 283 - DNA, 171 bp, Artificial Sequence
  Consensus sequence
gcaccatctcacaattgccagttaacgtcttccttctctctctgtcatagggactctggatcccagaaggtgagaaagttaaaattcccgtcgctatcaaggaattaagagaagcaacatctccgaaagccaacaaggaaatcctcgatgtgagtttctgctttgctgtgt

SEQ ID NO: 284 - DNA, 156 bp, Artificial Sequence
  Consensus sequence
gcaccatctcacaattgccagttaacgtcttccttctctctctgtcatagggactctggatcccagaaggtgagaaagttaaaattcccgtcgctatcaagacatctccgaaagccaacaaggaaatcctcgatgtgagtttctgctttgctgtgt

SEQ ID NO: 285 - DNA, 171 bp, Artificial Sequence; identical to SEQ ID NO: 283
  Consensus sequence

SEQ ID NO: 286 - DNA, 171 bp, Artificial Sequence; identical to SEQ ID NO: 283
  Consensus sequence

SEQ ID NO: 287 - DNA, 162 bp, Artificial Sequence
  Consensus sequence
gcaccatctcacaattgccagttaacgtcttccttctctctctgtcatagggactctggatcccagaaggtgagaaagttaaaattcccgtcgctatcaaggaaccaacatctccgaaagccaacaaggaaatcctcgatgtgagtttctgctttgctgtgt

SEQ ID NO: 288 - DNA, 171 bp, Artificial Sequence; identical to SEQ ID NO: 283
  Consensus sequence

SEQ ID NO: 289 - DNA, 148 bp, Artificial Sequence
  Consensus sequence
caccagggagtaactattcccagtcagaggcgctatgtgtattattatagctacctgttaaagaatcatctggattatagaccagtggcactgttgtttcacaagatgatgtttgaaactattccaatgttcagtggcggaacttgca

SEQ ID NO: 290 - DNA, 113 bp, Artificial Sequence
  Consensus sequence
caccagggagtaactattcccagtcagaggcgctatggattatagaccagtggcactgttgtttcacaagatgatgtttgaaactattccaatgttcagtggcggaacttgca

SEQ ID NO: 291 - DNA, 148 bp, Artificial Sequence; identical to SEQ ID NO: 289
  Consensus sequence

SEQ ID NO: 292 - DNA, 207 bp, Artificial Sequence
  Consensus sequence
gctgcagaaacatttggcacattccattcttaccaaactctaaattttctcttggaaactcccatttgagatcatattcatattctctgaaatcaacgtagaagtactcattatctgaggagccggtcacctgtaccatctgtagctggctttcatacctaaattgcttcagagatgaaatgatgagtcagttaggaataggcagtt

SEQ ID NO: 293 - DNA, 237 bp, Artificial Sequence
  Consensus sequence
gctgcagaaacatttggcacattccattcttaccaaactctaaattttctcttggaaactcccatttgagatcatattcatattctctgaaatcaacgtgatcatattcatattctctgaaatcaacgtagaagtactcattatctgaggagccggtcacctgtaccatctgtagctggctttcatacctaaattgcttcagagatgaaatgatgagtcagttaggaataggcagtt

SEQ ID NO: 294 - DNA, 207 bp, Artificial Sequence; identical to SEQ ID NO: 292
  Consensus sequence

SEQ ID NO: 295 - DNA, 207 bp, Artificial Sequence; identical to SEQ ID NO: 292
  Consensus sequence

SEQ ID NO: 296 - DNA, 226 bp, Artificial Sequence
  Consensus sequence
gctgcagaaacatttggcacattccattcttaccaaactctaaattttctcttggaaactcccatttgagatcatattcatattctctgaaatcaaattcatattctctgaaatcaatagaagtactcattatctgaggagccggtcacctgtaccatctgtagctggctttcatacctaaattgcttcagagatgaaatgatgagtcagttaggaataggcagtt

SEQ ID NO: 297 - DNA, 207 bp, Artificial Sequence; identical to SEQ ID NO: 292
  Consensus sequence

(19) United States
(12) Patent Application Publication
(10) Pub. No.: US 2020/0277661 A1
(43) Pub. Date: Sep. 3, 2020

(54) Methods And Systems For Detecting Genetic Mutations

(71) Applicant: SeqTech Diagnostics LLC, Nashville, TN (US)

(72) Inventor: Adam Platt, Nashville, TN (US)

(21) Appl. No.: 16/737,535

(22) Filed: Jan. 8, 2020

Related U.S. Application Data
(63) Continuation of application No. 15/113,293, filed on Jul. 21, 2016, now abandoned.
(60) Provisional application No. 61/930,063, filed on Jan. 22, 2014.

(51) Int. Cl. C12Q 1/6827; G16B 30/00; C12Q 1/6886

(52) U.S. Cl. CPC C12Q 1/6827; G16B 20/00; C12Q 1/6886; G16B 30/00

(57) ABSTRACT

Methods for detecting a genetic mutation in target nucleotide sequences by sorting the target nucleotide sequences into bins, aligning the target nucleotide sequences in each bin with reference nucleotide sequences, and quantifying the number of target nucleotide sequences that align with reference sequences. Systems and kits for detecting a genetic mutation in target nucleotide sequences.

### RELATED APPLICATION

This application is a Continuation of U.S. application Ser. No. 15/113,293, filed Jan. 21, 2015, which is the U.S. National Stage of International Application No. PCT/US2015/012273, filed Jan. 21, 2015, which designates the U.S., published in English, and claims the benefit of U.S. Provisional Application No. 61/930,063, filed on Jan. 22, 2014. The entire teachings of the above applications are incorporated herein by reference.

### INCORPORATION BY REFERENCE OF MATERIAL IN ASCII TEXT FILE

This application incorporates by reference the Sequence Listing contained in the following ASCII text file being submitted concurrently herewith:
a) File name: 51261000004_SEQUENCELISTING.txt, created Jan. 7, 2020, 55 KB in size.

### BACKGROUND OF THE INVENTION

DNA sequencing technology has advanced rapidly over the last two decades. This has resulted in an increased utilization of technology for producing an every growing catalog of annotated DNA sequence(1), (2). Currently, the dominant strategy for characterizing DNA sequences is massively parallel sequencing (MPS), which is also called next-generation sequencing (NGS), where long nucleotide polymers are sheared into small fragments that are then interrogated simultaneously by cycles of single-base-addition synthesis reactions. This produces millions of short sequence reads that mirror the sequence in the original molecules being studied. Computers then apply alignment algorithms to stitch the reads together into a consensus representation of the sequence of bases found in the original molecule.

The ever-expanding amount of annotated sequences available has resulted in the specific characterization of the genetic basis for an increasing number of diseases and other phenotypes of interest (3), (4), (5). For particular mutants, this has created a market for genotyping assays that can efficiently detect their presence in a cohort of individuals or tissues (6). These assays are designed with prior knowledge of the structure of the mutation(s) being targeted. Current genotyping assays narrow the scope of what is investigated down to only the genes, alleles, or loci that are relevant. In many cases this can mean designing an assay to detect a few specific mutations or even a single genetic alteration.

MPS has recently become a diagnostic platform due to its ability to cover a multitude of biomarkers simultaneously (7), (8), (9). MPS is particularly used for detecting mutations of less than about 5 base pairs. However, due to the relatively low number of contiguous bases, an MPS instrument, which is able to read at about less than 500 bases at a time, loses specificity when detecting longer insertions or deletions, leading to a high number of false positive mutation calls (10), (11), (12), (13). Generally, an MPS instrument will lose specificity in identifying insertions, including repetitions, or deletions that are longer than about 5-10% of the average read length of the MPS instrument being used to analyze the sample. The instrument needs the sequence read to cover enough bases (e.g., about 23) on both sides of the mutation to independently align each side to the reference sequence in order to reliably detect a mutation. For longer mutations there is less sequence to use for alignment on either side within a sequence read, making it harder for the instrument to align. Relaxing the statistical stringency of the alignment algorithm leads to a high prevalence of false positives. Thus, if an MPS instrument detects the insertion or deletion of a number of contiguous bases greater that are about 10% of the instrument average read length, the mutations need to be confirmed by another testing method.

Larger structural variations (e.g., greater than about 1,000 bases), involving sequences of DNA or RNA that are longer than the instrument's read length, pose additional issues (13), (14). Sequencing instruments identify mutations by aligning the segments of the read that fall on either side of the mutation. In cases where the mutation is larger than the read length there is no adjoining sequence to align because the entire read falls within the mutation. There are three analytical methods used to call these types of mutations using short-read sequencing data, including: (1) the read-depth approach, (2) the split-read approach, and (3) the read pair approach. None are particularly effective. For example, in one study only about 1.5% of the mutations in a sample were detected by all three of the read-depth, split-read, and read-pair methods, and only about 58.7% were detected by at least one of the methods. Similar studies have shown that less than about 50% of sequencing-based, structural variant calls can be verified by other methods, and the rest are false positives (13), (14). MPS has been paired with other methods (e.g., multiplex ligation-dependent probe amplification (MPLA)) to provide greater specificity of genotyping, but this approach has drawbacks, such as extra cost, time, and patient sample consumption. The assays themselves are also relatively complicated and difficult to validate for use in clinical settings.

Therefore, a need exists for improved systems and methods to detect various classes of mutations, including large structural variations, with high specificity limits.

### SUMMARY OF THE INVENTION

The invention generally is directed to methods, systems and kits for detecting a genetic mutation.

In one embodiment, the invention includes a method for detecting a genetic mutation, comprising the steps of a) obtaining a plurality of target nucleotide sequences from the products of one or more nucleic acid amplification reactions; b) sorting the target nucleotide sequences into a plurality of bins according to a sorting criterion; c) assigning a unique set of reference nucleotide sequences to each bin, wherein the reference nucleotide sequences include non-canonical reference sequences; d) aligning the target nucleotide sequences in each bin with the set of reference nucleotide sequences assigned to the bin; e) quantifying the number of target nucleotide sequences in a bin that align with each non-canonical reference sequence; and f) detecting a genetic mutation, wherein a target nucleotide sequence that aligns with a non-canonical reference sequence in a bin, a target nucleotide sequence that is present in an unexpected bin, or the absence of target nucleotide sequences in an expected bin is indicative of a genetic mutation.

In another embodiment, the invention includes an apparatus for detecting a genetic mutation, comprising a processor configured to a) receive sequence data comprising a plurality of target nucleotide sequences; b) sort the target nucleotide sequences into a plurality of bins according to a sorting criterion; c) generate and assign a unique set of reference nucleotide sequences to each bin, wherein the reference nucleotide sequences include non-canonical reference sequences; d) align the target nucleotide sequences in each bin with the set of reference nucleotide sequences assigned to the bin; e) quantify the number of target nucleotide sequences in a bin that align with each non-canonical reference sequence; and f) provide a user output indicating whether a genetic mutation is present in the target nucleotide sequence.

In an additional embodiment, the invention includes a method for detecting the presence of a genetic mutation that alters gene expression, comprising the steps of a) obtaining a plurality of target nucleotide sequences; b) aligning the target nucleotide sequences with a set of reference nucleotide sequences comprising a first reference sequence and at least one additional reference sequence; c) quantifying the number of target nucleotide sequences that align with each of the reference nucleotide sequences; and d) comparing the quantity of target nucleotide sequences that align with the first reference nucleotide sequence to the quantity of target nucleotide sequences that align with the other reference nucleotide sequences, wherein an increase or decrease in the quantity of target nucleotide sequences that align with the first reference nucleotide sequence relative to the quantity of target nucleotide sequences that align with the other reference nucleotide sequences is indicative of a genetic mutation that alters gene expression.

In a further embodiment, the invention includes a method for detecting a genetic mutation, comprising the steps of a) amplifying three or more target nucleotide sequences in a sample comprising genomic DNA to produce an amplicon for each target nucleotide sequence; b) sequencing the amplicons; and c) analyzing the sequences of the amplicons for the presence of a genetic mutation. In some embodiments, the three or more target nucleotide sequences include a) at least one target nucleotide sequence is being analyzed for a single nucleotide polymorphism (SNP), b) at least one target nucleotide sequence is being analyzed for an insertion, a deletion, or an insertion and a deletion, and c) at least one target nucleotide sequence is being analyzed for a rearrangement.

In yet another embodiment, the invention includes a kit for detecting a genetic mutation, comprising a first probe set comprising target-specific primers and a second probe set comprising sequencer-specific primers. In some embodiments, the first probe set comprises a) a pair of target-specific primers for detecting a single nucleotide polymorphism (SNP) in at least one target nucleotide sequence, b) a pair of target-specific primers for detecting an insertion, a deletion, or an insertion and a deletion in at least one target nucleotide sequence, and c) a pair of target-specific primers for detecting a rearrangement in at least one target nucleotide sequence.

The invention provides new methods, systems and kits for detecting a genetic mutation, for example, in a subject, such as a human subject, or organism. The invention has advantages over current methods, systems and kits to detect a genetic mutation. For example, the methods, systems and kits of the invention are useful for detecting different types of mutations of varying sizes in a single assay.

### BRIEF DESCRIPTION OF THE DRAWINGS

FIG. 1 summarizes current mutation-detection technologies, which are limited in the size of mutation that can be detected (e.g., small mutations (about 1 to about 20 bases), medium-sized mutations (about 21 to about 150 bases) or large mutations (greater than about 150 bases (e.g., about 300 bases, about 100,000 bases, about 100,000,000 bases)), but not a combination of small, medium and large mutations).

FIG. 2 is a flowchart of an exemplary genotype calling process for analyzing target nucleotide sequences for the presence of a genetic mutation.

FIG. 3A depicts Dummy Primer1 hybridizing to the positive (+) strand of chromosome 2 in Intron 13 of the EML4 gene on the coding strand for EML4, 50 base-pairs (bp) upstream (5′) of a known fusion point of EML4 and ALK. The genomic sequence downstream (3′) of Dummy Primer1 is italicized.

FIG. 3B depicts Dummy Primer2 also hybridizing to the positive strand of chromosome 2, roughly 12 million bp downstream of Dummy Primer1 in Intron 19 of the ALK gene on the non-coding strand for ALK. This primer falls 50 bp downstream of a known fusion point with EML4. The genomic sequence upstream of Dummy Primer2 is shown underlined.

FIG. 3C shows that, in normal, canonical (wt) DNA, Dummy Primer 1 and Dummy Primer 2 are not capable of initiating PCR amplification because both prime the positive strand and the primers are located too far apart from each other (about 12 Mb.) When particular genomic inversions occur, this is no longer the case. The intronic region where Dummy Primer 2 resides becomes the minus strand of chromosome 2, putting the two dummy primers in the correct orientation to generate PCR products that span the breakpoint.

FIG. 3D depicts the generation of a rearrangement hash. Fusion break-points have been reported to be located 50 bp away and exactly in between Dummy Primers1 and 2 but the actual location can vary slightly (plus or minus 50 bp) in a local scale or fall in a completely different pair of introns. In order to account for the local variance (plus or minus 50 bp) a unique set reference sequences is generated for each bin that covers each possible amplicon sequence that could result from each combination of dummy primers that are included in the PCR reaction. For a bin with 100 bp of sequence between Dummy Primers1 and 2, there are 99 possible amplicon sequences. The reference sequence that would match amplicons generated from a sample containing the breakpoint reported in the literature is shown in the middle of the table and contains 50 bp downstream of Dummy Primer1 and 50 bp upstream of Dummy Primer2. The full hash of reference sequences is generated iteratively by varying the amount of contiguous sequence included from each primer's flanking region while keeping the total length constant to match the bin the hash is being generated for (in this case 100.)

FIG. 4 is a histogram showing the expected distribution of amplicon read-length for the prototype assay described in the Table 5.

FIG. 5 shows the amplicon size distribution from the first pass of a 150×150 paired-end run on an Illumina MISEQ® desktop DNA sequencer.

FIG. 6 is a zoomed-in view of the histogram shown in FIG. 5.

FIG. 7 is a schematic showing the location of the two anchor amplicons and the two probe amplicons used to detect a large indel.

FIG. 8 illustrates how homozygous deletions, heterozygous deletions, no indel, heterozygous insertions, and homozygous insertions are predicted to affect the number and fraction of probe amplicons and anchor amplicons.

FIG. 9 illustrates how homozygous deletions, heterozygous deletions, no indel, heterozygous insertions, and homozygous insertions are predicted to affect the ratios of probe amplicons and anchor amplicons.

FIG. 10 shows the distribution of reads for a canonical sample and a sample homozygous for the GALC deletion. The lack of reads within the indel region is evident by the lack of probe sequence reads.

FIG. 11 shows the read numbers of anchor and probe amplicons in the sample with the CMT1A duplication compared to canonical.

FIG. 12 shows the ratios of probe to anchor amplicons in the sample with the CMT1A duplication compared to canonical.

FIG. 13 summarizes the genetic regions targeted by the single cancer test described in Example 3 herein covering 30 regions of 13 different genes that are known to potentially harbor somatic mutations of known or potential therapeutic value, and the most common mutations found in each target.

FIGS. 14A and 14B summarize embodiments of the invention, such as Amplicon Size, Expected Readlength, Primer Sequences and Exon Coverage of Small and Medium Mutations covered by the single cancer test described in Example 3 herein.

FIG. 15A shows detection of a canonical EGFR sequence in exon 19.

FIG. 15B shows detection of EGFR L747-A750del, which has a 15 base-pair (bp) deletion in exon 19 of EGFR.

FIG. 15C shows consensus reads and expected sequences for EGFR L747-A750del and its canonical counterpart.

FIG. 16A shows detection of a canonical EGFR sequence in exon 19.

FIG. 16B shows detection of EGFR L747-E749del, A750P, which has a 9 base pair deletion followed by a G to C substitution 4 base-pairs after the deletion in exon 19 of EGFR.

FIG. 16C shows consensus reads and expected sequences for EGFR L747-E749del, A750P and its canonical counterpart.

FIG. 17A shows detection of a canonical PTEN sequence.

FIG. 17B shows detection of PTEN c.524_558del35, which has a 35 base-pair (bp) deletion.

FIG. 17C shows consensus reads and expected sequences for PTEN c.524_558del35 and its canonical counterpart.

FIG. 18A shows detection of a canonical FLT3 sequence.

FIG. 18B shows detection of the same FLT-3 region in MV-4-11 cancer cell line, which has a 30 base-pair (bp) FLT3 internal-tandem duplication (ITD) insertion.

FIG. 18C shows consensus reads and expected sequences for the FLT3 ITD insertion and its canonical counterpart.

FIG. 19A shows detection of a canonical FLT3 sequence.

FIG. 19B shows detection of the same FLT-3 region in MOLM-13 cancer cell line, which has a 21 base-pair (bp) FLT3 internal-tandem duplication (ITD) insertion.

FIG. 19C shows consensus reads and expected sequences for the FLT3 ITD insertion and its canonical counterpart.

### DETAILED DESCRIPTION OF THE INVENTION

The features and other details of the invention, either as steps of the invention or as combinations of parts of the invention, will now be more particularly described and pointed out in the claims. It will be understood that the particular embodiments of the invention are shown by way of illustration and not as limitations of the invention. The principal features of this invention can be employed in various embodiments without departing from the scope of the invention.

The invention generally is directed to the area of nucleic acid sequencing, in particular methods, systems and kits for detecting genetic mutations. In embodiments, the invention generally is directed to analytic steps for analyzing sequencing data to detect the presence of mutations of various types including, for example, SNPs, indels, structural variations, inversions, rearrangements, duplications and Copy-Number-Variations, as well as instances of aberrant gene expression levels.

The invention includes methods for detecting genetic mutations. The methods described herein can be useful in the detection of a variety of genetic mutations. Mutations that can be detected using the methods described herein include, for example, a single nucleotide polymorphism (SNP), an insertion, a deletion, a tandem duplication, and a rearrangement (e.g., an inversion, a translocation), as well as any combination of the foregoing. The genetic mutation can be a germline mutation or a somatic mutation. Typically, the mutation is a known mutation. For example, the mutation can be a recurrent mutation that has been associated with one or more cancers.

In an embodiment, the invention is directed to a method for detecting a genetic mutation, comprising the steps of a) obtaining a plurality of target nucleotide sequences; b) sorting the target nucleotide sequences into a plurality of bins according to a sorting criterion; c) assigning a unique set of reference nucleotide sequences to each bin, wherein the reference nucleotide sequences include non-canonical reference sequences; d) aligning the target nucleotide sequences in each bin with the set of reference nucleotide sequences assigned to the bin; e) quantifying the number of target nucleotide sequences in a bin that align with each non-canonical reference sequence; and f) detecting a genetic mutation, wherein a target nucleotide sequence that aligns with a non-canonical reference sequence in a bin, a target nucleotide sequence that is present in an unexpected bin, or the absence of target nucleotide sequences in an expected bin is indicative of a genetic mutation.

As used herein, “target nucleotide sequence” refers to a sequence of contiguous nucleotides in a nucleic acid molecule that is being analyzed for the presence of a genetic mutation. The target nucleotide sequence can be known to have a mutation, suspected of having a mutation, or be tested for a mutation without knowledge or suspicion as to whether a mutation is present. The nucleic acid molecule employed in the methods, systems and kits described herein can be genomic DNA, cDNA or RNA. In a particular embodiment, the nucleic acid molecule is human genomic DNA.

The nucleic acid molecule can be isolated from a biological source (e.g., a human) employing routine techniques. Biological sources of nucleic acid molecules include nucleic acid molecules extracted from cells, tissues, bodily fluids, and organs. In a particular embodiment, the biological source is a tissue biopsy (e.g., a tumor biopsy). In another embodiment, the biological source is a bodily fluid (e.g., blood, bone marrow, plasma, serum, spinal fluid, lymph fluid, tears, saliva, mucus, sputum, urine, fecal matter, semen, and amniotic fluid). In an additional embodiment, the biological source is a maternal sample that includes fetal DNA.

In general, a target nucleotide sequence that is being analyzed using a method described herein will have a length of about 50 to about 500 nucleotides. For example, a target nucleotide sequence can have a length of about 50, about 100, about 150, about 200, about 250, about 300, about 350, about 400, about 450, or about 500 nucleotides.

In some embodiments of the invention, the target nucleotide sequences being analyzed are obtained from the products of one or more nucleic acid amplification reactions. One of ordinary skill in the art would understand that the products of such reactions are referred to as amplicons. A variety of nucleic acid amplification reactions are known in the art. In one embodiment, a polymerase chain reaction (PCR) is used to amplify target nucleic acid molecules. Examples of polymerase chain reactions include multiplex polymerase chain reactions and single-plex polymerase chain reactions. In some embodiments, the nucleic acid amplification reaction includes primers (e.g., dummy primers) that are designed to produce an amplification product only if a mutation (e.g., a rearrangement) is present. The term “dummy primers” refers to a pair of nucleic acid amplification primers that will not produce an amplicon unless there is a structural variation in the target nucleotide sequence. Exemplary dummy primer sequences are disclosed in Tables 9 and 10.

In some embodiments, the target nucleotide sequences can be obtained from one or more amplicons with the aid of a sequencer instrument. A variety of sequencers are commercially available. In an embodiment, the sequencer is a Next Generation Sequencer (NGS).

The plurality of target nucleotide sequences that are being analyzed in the invention can include unaligned sequences, paired sequences and/or unpaired sequences. In a particular embodiment, the plurality of target nucleotide sequences include paired sequences. The terms “paired sequences” or “paired-end sequences” refer to two nucleotide sequence reads that begin at (2) at opposite ends of a single nucleic acid molecule that is being analyzed. For example, some sequence instruments are capable of first reading the first 50-300 bases on the 5′ end of a DNA molecule before copying the whole molecule to create a reverse complement of the original molecule and then reading from the 5′ end of the new molecule which corresponds to the 3′ of the original molecule. This results in a pair of reads that each start at opposite ends of the DNA molecule being sequenced. In some embodiments, a target PCR reaction is used to create amplicons that are shorter than 2× the read length of each of the reads so that there is some overlap between the pairs. This allows for very accurate gauging of the length of the molecules being sequenced.

After a plurality of target nucleotide sequences have been obtained, the target nucleotide sequences are sorted into a plurality of bins according to a sorting criterion (e.g., one or more sorting criteria). The term “sorting criterion” refers to a particular feature or set of features that are used to sort target nucleotide sequences into bins. Exemplary features include a defined sequence length, the presence of a particular nucleotide sequence within a target sequence, and the absence of a particular nucleotide sequence in a target sequence. For example, the feature can be a unique sequence, such as a “barcode.” The barcode sequence can be, e.g., the sequence of a target-specific primer, or can be included in a target-specific primer sequence. The barcode sequence can be engineered onto one or both ends of a target nucleotide sequence, for example, during an amplification reaction. In general, the unique sequence will be about 3-50 nucleotides in length, for example, about 3 to about 10 nucleotides, about 18 to about 33 nucleotides or about 21 to about 43 nucleotides.

As used herein, “bin” refers to a data (e.g., binary data) container used to store at least one file (e.g., a sequence file) selected from the group consisting of a computer-readable file and a human-readable file, or a combination thereof, that includes at least one sequence of nucleotides. Sequences within a bin share a common feature or features including, for example, at least one feature selected from the group consisting of sequence length and a specific nucleotide sequence, or a combination thereof. For example, the sequences in a bin can start, end, or start and end, with a specific sequence of nucleotides (e.g., a barcode). A bin can be distinguished from at least one other bin based on the common feature or features that are possessed by each nucleotide sequence within the bin.

A “reference nucleotide sequence” refers to a pre-determined, pre-generated nucleotide sequence that is stored in a hash of reference nucleotide sequences that has been assigned to a bin. The reference nucleotide sequences are intended for alignment with target nucleotide sequences that have been sorted into the same bin. A reference nucleotide sequence can be a canonical nucleotide sequence (i.e., a consensus nucleotide sequence in a reference human genome) or a non-canonical nucleotide sequence (i.e., a variant of a canonical nucleotide sequence). In an embodiment, a unique set of reference nucleotide sequences is assigned to each bin, such that no two bins include the same set of reference sequences. In some embodiments (e.g., in a SNP hash), a set of reference nucleotide sequences will include both canonical (e.g., a single canonical nucleotide sequence) and non-canonical nucleotide sequences (e.g., several non-canonical sequences). Generally, a bin contains an excess of non-canonical sequences compared to canonical sequences. In other embodiments (e.g., in an indel hash or rearrangement hash), a set of reference nucleotide sequences includes only non-canonical nucleotide sequences. The set of reference nucleotide sequences in each bin can vary in number and depends, in part, on the length of the sequence being analyzed. In general, a bin includes more than about 100 different reference nucleotide sequences (e.g., greater than about 50,000 reference nucleotide sequences).

In one embodiment, the plurality of bins includes a bin comprising a SNP hash of reference nucleotide sequences. The term “SNP hash” refers to a set of reference nucleotide sequences of identical length comprising a single canonical reference sequence and a plurality of non-canonical reference sequences having 1, 2, 3, 4 or 5 single nucleotide substitutions relative to the canonical nucleotide sequence. In a particular embodiment, the SNP hash includes non-canonical reference sequences representing each possible variant containing 1, 2, 3, 4 or 5 single nucleotide substitutions of a single canonical reference sequence. The generation of exemplary SNP hashes for a particular canonical reference sequence is shown in Tables 1 and 2.

TABLE 1
Generation of a SNP Hash of reference nucleotide sequences containing a
single error or deviation from the canonical reference (deviations from canonical
are underlined).
Creation of sequences with single base pair (bp) difference from canonical

| Reference | Sequence |
|---|---|
| Canonical | ATTGAGGATGTAGGACTCCCAGCTAAAACTGCCTTCTGCCCA |
| | (SEQ ID NO: 1) |
| Alt_1C | CTTGAGGATGTAGGACTCCCAGCTAAAACTGCCTTCTGCCCA |
| | (SEQ ID NO: 2) |
| Alt_1T | TTTGAGGATGTAGGACTCCCAGCTAAAACTGCCTTCTGCCCA |
| | (SEQ ID NO: 3) |
| Alt_1G | GTTGAGGATGTAGGACTCCCAGCTAAAACTGCCTTCTGCCCA |
| | (SEQ ID NO: 4) |
| Alt_2A | AATGAGGATGTAGGACTCCCAGCTAAAACTGCCTTCTGCCCA |
| | (SEQ ID NO: 5) |
| Alt_2C | ACTGAGGATGTAGGACTCCCAGCTAAAACTGCCTTCTGCCCA |
| | (SEQ ID NO: 6) |
| Alt_2G | AGTGAGGATGTAGGACTCCCAGCTAAAACTGCCTTCTGCCCA |
| | (SEQ ID NO: 7) |
| Alt_3A | ATAGAGGATGTAGGACTCCCAGCTAAAACTGCCTTCTGCCCA |
| | (SEQ ID NO: 8) |
| Alt_3C | ATCGAGGATGTAGGACTCCCAGCTAAAACTGCCTTCTGCCCA |
| | (SEQ ID NO: 9) |
| Alt_3G | ATGGAGGATGTAGGACTCCCAGCTAAAACTGCCTTCTGCCCA |
| | (SEQ ID NO: 10) |
| Alt_4A | ATTAAGGATGTAGGACTCCCAGCTAAAACTGCCTTCTGCCCA |
| | (SEQ ID NO:11) |
| Alt_4C | ATTCAGGATGTAGGACTCCCAGCTAAAACTGCCTTCTGCCCA |
| | (SEQ ID NO: 12) |
| Alt_4T | ATTTAGGATGTAGGACTCCCAGCTAAAACTGCCTTCTGCCCA |
| | (SEQ ID NO: 13) |
| Alt_NN | Repeat to generate multiple iterations, including exhaustive iterations |

The process used to generate the sequences in Table 1 can be repeated to generate additional reads with 1 deviation from the reference.

TABLE 2
Generation of a SNP Hash of reference nucleotide sequences containing
two errors or deviations from the canonical (deviations from canonical
are underlined).
Creation of sequences with two base pair (bp) differences from canonical

| | |
|---|---|
| Alt_1C | CTTGAGGATGTAGGACTCCCAGCTAAAACTGCCTTCTGCCCA |
| | (SEQ ID NO: 14) |
| Alt_1C_2A | CATGAGGATGTAGGACTCCCAGCTAAAACTGCCTTCTGCCCA |
| | (SEQ ID NO: 15) |
| Alt_1C_2C | CCTGAGGATGTAGGACTCCCAGCTAAAACTGCCTTCTGCCCA |
| | (SEQ ID NO: 16) |
| Alt_1C_2G | CGTGAGGATGTAGGACTCCCAGCTAAAACTGCCTTCTGCCCA |
| | (SEQ ID NO: 17) |
| Alt_1C_3A | CTAGAGGATGTAGGACTCCCAGCTAAAACTGCCTTCTGCCCA |
| | (SEQ ID NO: 18) |
| Alt_1C_3C | CTCGAGGATGTAGGACTCCCAGCTAAAACTGCCTTCTGCCCA |
| | (SEQ ID NO: 19) |
| Alt_1C_3G | CTGGAGGATGTAGGACTCCCAGCTAAAACTGCCTTCTGCCCA |
| | (SEQ ID NO: 20) |
| Alt_1C_4A | CTTAAGGATGTAGGACTCCCAGCTAAAACTGCCTTCTGCCCA |
| | (SEQ ID NO: 21) |
| Alt_1C_4C | CTTCAGGATGTAGGACTCCCAGCTAAAACTGCCTTCTGCCCA |
| | (SEQ ID NO: 22) |
| Alt_1C_4T | CTTTAGGATGTAGGACTCCCAGCTAAAACTGCCTTCTGCCCA |
| | (SEQ ID NO: 23) |
| Alt_1C_5C | CTTGCGGATGTAGGACTCCCAGCTAAAACTGCCTTCTGCCCA |
| | (SEQ ID NO: 24) |
| Alt_1C_5T | CTTGAGGATGTAGGACTCCCAGCTAAAACTGCCTTCTGCCCA |
| | (SEQ ID NO: 25) |
| Alt_1C_5G | CTTGAGGATGTAGGACTCCCAGCTAAAACTGCCTTCTGCCCA |
| | (SEQ ID NO: 26) |
| Alt_NN_NN | Repeat to generate multiple iterations, including exhaustive iterations |

The process used to generate the sequences in Table 2 can be repeated to generate additional reads with 2 deviations from the reference and can be continued to generate additional reads with 3 deviations, then 4 deviations, etc.

In another embodiment, the plurality of bins includes a bin that includes an indel hash of reference nucleotide sequences. “Indel,” as used herein, refers to a deletion, an insertion, a combination of one or more deletions and one or more insertions, or a nucleotide sequence comprising both an insertion and a deletion (e.g., a nucleotide sequence in which 10 bases are deleted and a different sequence of 5 bases are inserted in its place) of nucleotides in a nucleotide sequence. As used herein, “indel hash” refers to a set of reference nucleotide sequences of identical length comprising non-canonical reference sequences that differ from a single canonical reference sequence by the addition and/or deletion of a defined number of nucleotides (e.g., a number of nucleotides in the range of about 1 to about 450 nucleotides). In a particular embodiment, the indel hash includes non-canonical reference sequences representing each possible variant containing an insertion or a deletion of a specified number of nucleotides in a single canonical reference sequence.

The generation of an exemplary indel hash for a particular canonical reference sequence is shown in Table 3. The reference sequences in Table 3 are generated for a bin that is 2 bp longer than an amplicon that is expected to be present in the reaction. This is done by systematically adding combinations of 2 bases to every position in the read, shown underlined. This is repeated for each amplicon expected to be in the reaction, adjusting the expected sequences of the amplicons to match the bin by either inserting or removing the appropriate number of bases. The process is repeated for every bin in the analysis.

TABLE 3
Generation of sequence references for an Indel Hash of reference
nucleotide sequences.
Creation of Indel reference sequences for a bin 2 bp longer than an
expected amplicon

| Reference | Sequence |
|---|---|
| Canonical | ATTGAGGATGTAGGACTCCCAGCTAAAACTGCCTTCTGCCCA |
| | (SEQ ID NO: 27) |
| Alt_Pos0_VarAA | AAATTGAGGATGTAGGACTCCCAGCTAAAACTGCCTTCTGCCCA |
| | (SEQ ID NO: 28) |
| Alt_Pos0_VarAT | ATATTGAGGATGTAGGACTCCCAGCTAAAACTGCCTTCTGCCCA |
| | (SEQ ID NO: 29) |
| Alt_Pos0_VarAG | AGATTGAGGATGTAGGACTCCCAGCTAAAACTGCCTTCTGCCCA |
| | (SEQ ID NO: 30) |
| Alt_Pos0_VarAC | ACATTGAGGATGTAGGACTCCCAGCTAAAACTGCCTTCTGCCCA |
| | (SEQ ID NO: 31) |
| Alt_Pos0_VarTA | TAATTGAGGATGTAGGACTCCCAGCTAAAACTGCCTTCTGCCCA |
| | (SEQ ID NO: 32) |
| Alt_Pos0_VarTT | TTATTGAGGATGTAGGACTCCCAGCTAAAACTGCCTTCTGCCCA |
| | (SEQ ID NO: 33) |
| Alt_Pos0_VarTG | TGATTGAGGATGTAGGACTCCCAGCTAAAACTGCCTTCTGCCCA |
| | (SEQ ID NO: 34) |
| Alt_Pos0_VarTC | TCATTGAGGATGTAGGACTCCCAGCTAAAACTGCCTTCTGCCCA |
| | (SEQ ID NO: 35) |
| Alt_Pos0_VarGA | GAATTGAGGATGTAGGACTCCCAGCTAAAACTGCCTTCTGCCCA |
| | (SEQ ID NO: 36) |
| Alt_Pos0_VarGT | GTATTGAGGATGTAGGACTCCCAGCTAAAACTGCCTTCTGCCCA |
| | (SEQ ID NO: 37) |
| Alt_Pos0_VarGG | GGATTGAGGATGTAGGACTCCCAGCTAAAACTGCCTTCTGCCCA |
| | (SEQ ID NO: 38) |
| Alt_Pos0_VarGC | GCATTGAGGATGTAGGACTCCCAGCTAAAACTGCCTTCTGCCCA |
| | (SEQ ID NO: 39) |
| Alt_Pos0_VarCA | CAATTGAGGATGTAGGACTCCCAGCTAAAACTGCCTTCTGCCCA |
| | (SEQ ID NO: 40) |
| Alt_Pos0_VarCT | CTATTGAGGATGTAGGACTCCCAGCTAAAACTGCCTTCTGCCCA |
| | (SEQ ID NO: 41) |
| Alt_Pos0_VarCG | CGATTGAGGATGTAGGACTCCCAGCTAAAACTGCCTTCTGCCCA |
| | (SEQ ID NO: 42) |
| Alt_Pos0_VarCC | CCATTGAGGATGTAGGACTCCCAGCTAAAACTGCCTTCTGCCCA |
| | (SEQ ID NO: 43) |
| Alt_Pos1_VarAA | AAATTGAGGATGTAGGACTCCCAGCTAAAACTGCCTTCTGCCCA |
| | (SEQ ID NO: 44) |
| Alt_Pos1_VarAT | AATTTGAGGATGTAGGACTCCCAGCTAAAACTGCCTTCTGCCCA |
| | (SEQ ID NO: 45) |
| Alt_Pos1_VarAG | AAGTTGAGGATGTAGGACTCCCAGCTAAAACTGCCTTCTGCCCA |
| | (SEQ ID NO: 46) |
| Alt_Pos1_VarAC | AACTTGAGGATGTAGGACTCCCAGCTAAAACTGCCTTCTGCCCA |
| | (SEQ ID NO: 47) |
| Alt_Pos1_VarTA | ATATTGAGGATGTAGGACTCCCAGCTAAAACTGCCTTCTGCCCA |
| | (SEQ ID NO: 48) |
| Alt_Pos1_VarTT | ATTTTGAGGATGTAGGACTCCCAGCTAAAACTGCCTTCTGCCCA |
| | (SEQ ID NO: 49) |
| Alt_Pos1_VarTG | ATGTTGAGGATGTAGGACTCCCAGCTAAAACTGCCTTCTGCCCA |
| | (SEQ ID NO: 50) |
| Alt_Pos1_VarTC | ATCTTGAGGATGTAGGACTCCCAGCTAAAACTGCCTTCTGCCCA |
| | (SEQ ID NO: 51) |
| Alt_Pos1_VarGA | AGATTGAGGATGTAGGACTCCCAGCTAAAACTGCCTTCTGCCCA |
| | (SEQ ID NO: 52) |
| Alt_Pos1_VarGT | AGTTTGAGGATGTAGGACTCCCAGCTAAAACTGCCTTCTGCCCA |
| | (SEQ ID NO: 53) |
| Alt_Pos1_VarGG | AGGTTGAGGATGTAGGACTCCCAGCTAAAACTGCCTTCTGCCCA |
| | (SEQ ID NO: 54) |
| Alt_Pos1_VarGC | AGCTTGAGGATGTAGGACTCCCAGCTAAAACTGCCTTCTGCCCA |
| | (SEQ ID NO: 55) |
| Alt_Pos1_VarCA | ACATTGAGGATGTAGGACTCCCAGCTAAAACTGCCTTCTGCCCA |
| | (SEQ ID NO: 56) |
| Alt_Pos1_VarCT | ACTTTGAGGATGTAGGACTCCCAGCTAAAACTGCCTTCTGCCCA |
| | (SEQ ID NO: 57) |
| Alt_Pos1_VarCG | ACGTTGAGGATGTAGGACTCCCAGCTAAAACTGCCTTCTGCCCA |
| | (SEQ ID NO: 58) |
| Alt_Pos1_VarCC | ACCTTGAGGATGTAGGACTCCCAGCTAAAACTGCCTTCTGCCCA |
| | (SEQ ID NO: 59) |
| Alt_Pos2_VarNN | ATNNTGAGGATGTAGGACTCCCAGCTAAAACTGCCTTCTGCCCA |
| | (SEQ ID NO: 60) |
| Alt_Pos3_VarNN | ATTNNGAGGATGTAGGACTCCCAGCTAAAACTGCCTTCTGCCCA |
| | (SEQ ID NO: 61) |
| Alt_Pos4_VarNN | ATTGNNAGGATGTAGGACTCCCAGCTAAAACTGCCTTCTGCCCA |
| | (SEQ ID NO: 62) |
| Alt_Pos5_VarNN | ATTGANNGGATGTAGGACTCCCAGCTAAAACTGCCTTCTGCCCA |
| | (SEQ ID NO: 63) |
| Alt_Pos6_VarNN | ATTGAGNNGATGTAGGACTCCCAGCTAAAACTGCCTTCTGCCCA |
| | (SEQ ID NO: 64) |
| Alt_Pos7_VarNN | ATTGAGGNNATGTAGGACTCCCAGCTAAAACTGCCTTCTGCCCA |
| | (SEQ ID NO: 65) |
| Alt_Pos8_VarNN | ATTGAGGANNTGTAGGACTCCCAGCTAAAACTGCCTTCTGCCCA |
| | (SEQ ID NO: 66) |
| Alt_PosN_VarNN | Repeat to generate multiple iterations, including exhaustive iterations |

In another embodiment, the plurality of bins includes a bin comprising a rearrangement hash of reference nucleotide sequences. The term “rearrangement hash” refers to a set of reference nucleotide sequences comprising non-canonical reference sequences that each differ from a single canonical reference sequence by the addition, deletion or inversion of more than 100 contiguous nucleotides.

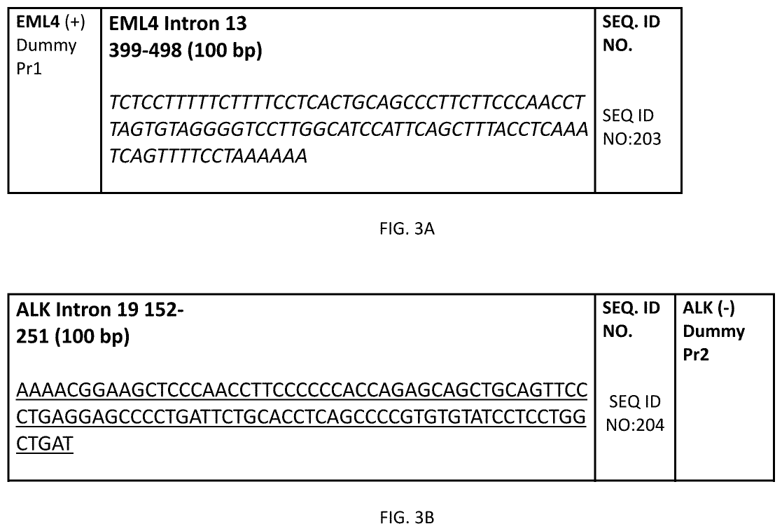

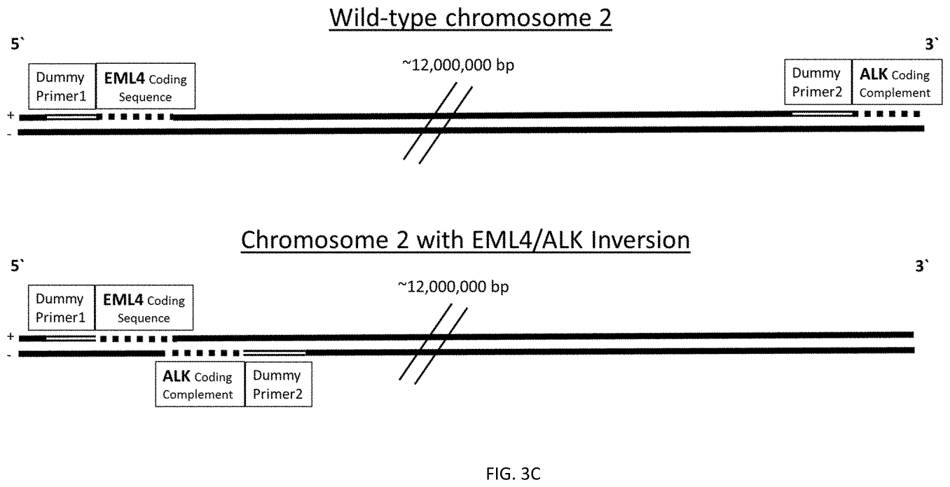

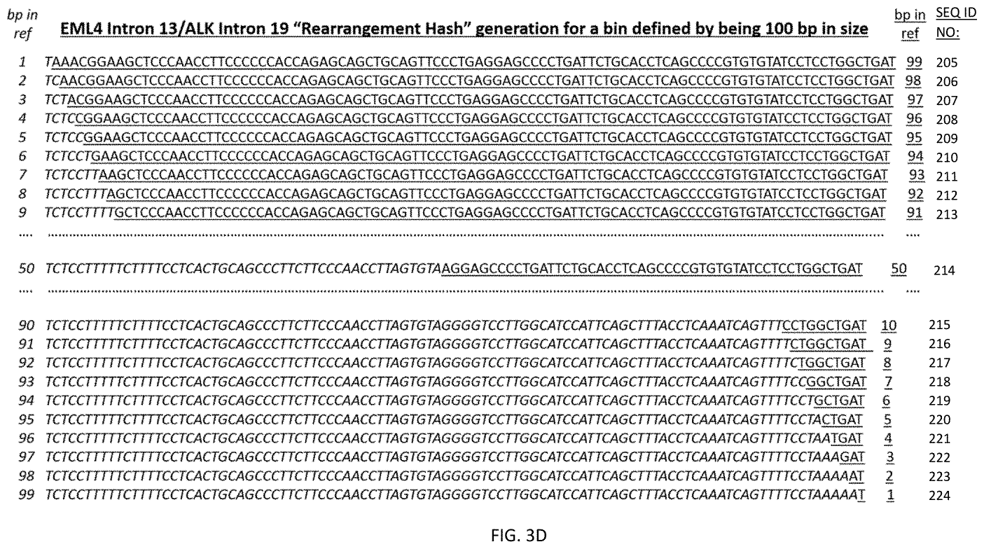

The generation of an exemplary rearrangement hash is shown in FIG. 3D. In general, the set of reference nucleotide sequences for a rearrangement hash of a bin can be generated by iteratively combining the sequence 3′ of a dummy primer with the sequence 5′ of every other dummy primer, as described herein. The amount sequence flanking each primer is iteratively varied but always includes a total number of bp that matches the size of the bin. For example, for a 150 base pair bin, the Rearrangement Hash would include reference sequences that combine 1 bp of the sequence immediately 3′ of Dummy PrimerA with 149 bp of the sequence 5′ of Dummy PrimerB, 2 bp of the sequence 3′ of Dummy PrimerA with 148 bp of the sequence 5′ of Dummy PrimerB, 3 bp of sequence 3′ of Dummy PrimerA with 147 bp of sequence 5′ of Dummy PrimerB, 4 bp of sequence 3′ of Dummy PrimerA with 146 bp of sequence 5′ of Dummy PrimerB, etc. This process is performed for each bin for every Dummy Primer in relation to every other Dummy Primer included in the reaction. The presence of rearrangement mutations in the nucleic acid template is inferred by a significant number of reads aligning to sequences in the rearrangement hash.

In a preferred embodiment, the plurality of bins includes a bin comprising a SNP hash of reference nucleotide sequences, a bin comprising an indel hash of reference nucleotide sequences and a bin comprising a rearrangement hash of reference nucleotide sequences.

Once bins have been established, the target nucleotide sequences in each bin are aligned with the set of reference nucleotide sequences in the bin. A variety of suitable algorithms for performing nucleotide sequence alignments are known in the art. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., Current Protocols in Molecular Biology).

One of ordinary skill in the art will understand that two sequences can align with one another without being identical (i.e., completely aligning, or having 100% identity). For example, two sequences can align with one another when there is at least about 70%, about 75%, about 80%, about 85%, about 90%, about 95%, about 99% or about 100% identity in the aligned portion(s) of the sequences. In some embodiments, a target nucleotide sequence and a reference sequence substantially align with one another. As used herein, “substantially aligns” refers to a target nucleotide sequence and a reference sequence that align with 0-5 nucleotide differences in the aligned portion(s) of the sequences.

The extent of alignment between a target sequence and a reference sequence that is indicative of the presence of a mutation in the target sequence depends, in part, on the type of mutation that is being detected. For example, in substitution mutations (e.g., SNPs), a target nucleotide sequence that completely aligns with 0 nucleotide deviations (i.e., 100% alignment) to a non-canonical reference sequence is indicative of the presence of a substitution mutation in the target sequence.

In the case of insertion and deletion mutations, the presence of the mutation is indicated primarily by a deviation in size (i.e., length) from a canonical reference sequence. For example, in insertion mutations, a target nucleotide sequence having a non-aligning segment of contiguous nucleotides that is flanked on one or both sides by, for example, at least about 18 contiguous bases that align with a reference sequence (e.g., with less than two errors per about 18 bases) is indicative of the presence of an insertion in the target sequence.

In an embodiment, for deletions, a target nucleotide sequence having two segments of, for example, at least about 18 contiguous nucleotides that align with the ends of a reference sequence (e.g., with less than two errors per about 18 bases), wherein the reference sequence also includes a middle segment of contiguous nucleotides that is absent from the target nucleotide sequence, is indicative of the presence of a deletion in the target sequence.

In another embodiment, for larger mutations (e.g., inversions, structural variations or translocations), a target nucleotide sequence having a first segment of, for example, at least about 18 contiguous nucleotides that aligns with a dummy primer sequence and the sequence that flanks the dummy primer (e.g., with less than 2 errors per about 18 bases of sequence) and second segment of at least about 18 base pairs that aligns with a second dummy primer, or the reverse complement of a second dummy primer, is indicative of the presence of a larger mutation in the target sequence.

In yet another embodiment, for mutations affecting gene expression levels, the alignment of, for example, at least about 18 bases of sequence with less than one error per about 18 bases is indicative of the presence of the mutation.

In embodiments, the number of target nucleotide sequences in a bin that align with each non-canonical reference sequence is quantified (e.g., the number of target and reference sequences that align are counted).

In other embodiments, an increase in the number of target nucleotide sequences that align (e.g., with 100% alignment) with non-canonical reference sequences in a bin compared to a background number is indicative of a genetic mutation in one or more target sequences. As used herein, “background number” refers to the number of target nucleotide sequences that align to the complete set of reference nucleotide sequences in a bin.

Once the target nucleotide sequences are sorted into bins and aligned with reference sequences, the presence of a genetic mutation can be detected. In one embodiment, a genetic mutation is detected by identifying a target nucleotide sequence that aligns with a non-canonical reference sequence in a bin. In another embodiment, a genetic mutation is detected by identifying a target nucleotide sequence that is present in an unexpected bin. The term “unexpected bin” refers to a bin that is defined by a feature (e.g., a sequence length or sequence identity) that is not expected to be present in the plurality of target nucleotide sequences.

In yet another embodiment, a genetic mutation is detected by identifying the absence of target nucleotide sequences in an expected bin. As used herein, “expected bin” refers to a bin that is defined by a feature (e.g., a sequence length or sequence identity) that is expected to be present in one or more target nucleotide sequences in the plurality of target nucleotide sequences.

In some embodiments, for example, when a given target nucleotide sequence does not align with any reference sequence in a bin, the target sequence can be moved to another bin and aligned with the reference sequences therein in an effort to identify the nature of the mutation.

When a target nucleotide sequence is determined to contain a mutation, the identity of that mutation can then be determined, if desired, by identifying the particular non-canonical reference sequence with which the target nucleotide sequence aligns.

In various embodiments, the method can further comprise one or more additional, optional steps. For example, the method can further comprise filtering the target nucleotide sequences for quality prior to sorting and aligning them. Methods of filtering nucleotide sequences for quality are known in the art.

Preferably, the method employs a computer (e.g., is computer-implemented). In a particular embodiment, the method is both computer-implemented and automated.

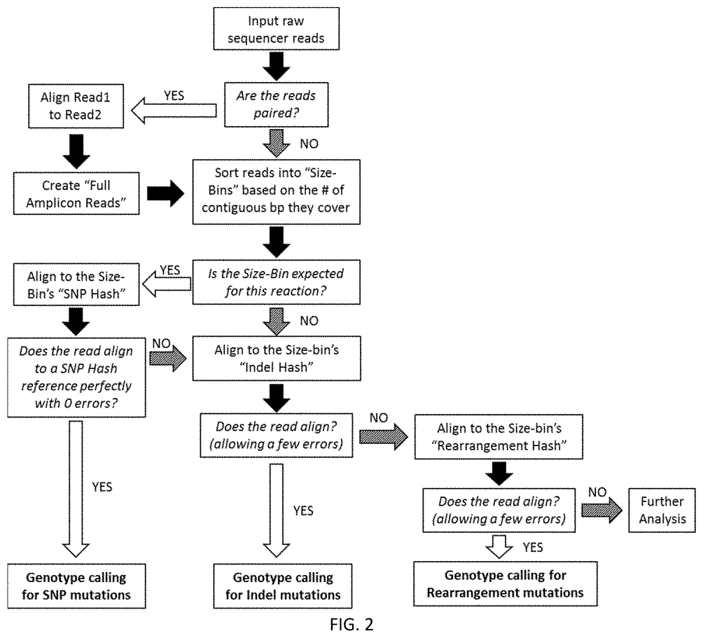

A flowchart for an exemplary method for analyzing target nucleotide sequences for the presence of a genetic mutation is shown in FIG. 2. As shown in FIG. 2, an exemplary genotype calling process is initiated with unaligned reads generated by a sequencer. If the reads are paired, each read is aligned to its companion and the complementary sequence contained by its companion is used to extend the read creating “Full Amplicon Reads” that match the full sequence of the original molecules that they were derived from. Non-paired reads and “Full Amplicon Reads” are then sorted into bins based on how long they are or how many contiguous bases they contain. The reads in each bin are then stringently aligned (a sequence reads is considered aligned if it contains 0 deviations from the reference) to the reference sequences in the SNP Hash (which contains the expected sequence and variants of that sequence that contain 1, 2, 3, 4, or 5 deviations from the expected, canonical sequence.) SNPs are detected by the presence of a significantly elevated number of reads aligning to non-canonical reference sequences compared to the canonical reference sequence in the SNP Hash.

An exemplary approach to detect the presence of other types of mutations (e.g., indels, rearrangements) is a multi-tiered approach. In one embodiment, each aggregated sequence that differs from the canonical reference is first compared to a set of known predetermined variant sequences ascertained from public databases, such as COSMIC. If the target sequence does not match a list of known variant sequences, then the target sequence is compared to a pre-computed subset of variants for the given target sequence. Generally, only a subset of possible genetic alterations is used.

For example, reads that fall in Unexpected Bins and Reads that fall into Expected Bins but do not align to any reads in the SNP Hash are then aligned (e.g., with leniency) to the references in the Indel Hash which contains variants of the canonical reference sequences for every Expected bin but with bases are added or subtracted to make the Canonical Reference sequences match the size of the Unexpected bin being analyzed. Indels are detected first by the presence of an Unexpected bin and then by presence of a significantly elevated number of reads aligning to references in the Indel Hash. The remaining reads that did not align to any sequences in either the SNP Hash or the Indel Hash are then aligned (with leniency) to the sequences in the Rearrangement Hash, which includes non-canonical sequences having a size defined by combining the sequence 3′ of each Dummy Primer included in the reaction with the sequence 5′ of any other Dummy Primer included in the reaction. Rearrangement mutations are detected by searching for reads in yet another bin—the bin that is set aside before merging the paired-end reads into longer overlapping sequences. A rearrangement is determined to be present if the target sequence starts with an expected sequence, but includes one or more additional unexpected sequences that do not match the expected sequences. Finally, any remaining reads that have not aligned to any of the Alignment Hashes are aligned to the full human genome using standard bioinformatics tools to understand their aberrant origin (e.g., by performing a global pairwise alignment using the Needleman-Wunsch algorithm to compare the alternate sequence to the expected, canonical reference sequence).

In another embodiment, the invention relates to an apparatus for detecting a genetic mutation, comprising a processor configured to a) receive sequence data comprising a plurality of target nucleotide sequences; b) sort the target nucleotide sequences into a plurality of bins according to a sorting criterion; c) generate and assign a unique set of reference nucleotide sequences to each bin, wherein the reference nucleotide sequences include non-canonical reference sequences; d) align the target nucleotide sequences in each bin with the set of reference nucleotide sequences assigned to the bin; e) quantify the number of target nucleotide sequences in a bin that align with each non-canonical reference sequence; and f) provide a user output indicating whether a genetic mutation is present in the target nucleotide sequence.

In a particular embodiment, the apparatus is a computer. In another embodiment, the apparatus includes multiple computers (e.g., 10 computers, each with 8 processors).

The apparatus can have one processor or multiple processors. The processor can be any suitable computer processor. The computer processor can be a single, dual, triple or quad core processor. In one embodiment the processor is a microprocessor. Typically, the processor is configured to run software comprising instructions for performing the steps of a sequence analysis algorithm.

In one embodiment, the processor is additionally configured to identify the genetic mutation in a target nucleotide sequence. In another embodiment, the processor is configured to identify target nucleotide sequences that do not align with a reference sequence in a bin and align those target nucleotide sequences with reference sequences in another bin.

In general, both the target nucleotide sequences and reference nucleotide sequences are stored on a computer-readable medium. Typically, the reference nucleotide sequences are generated and stored on a computer-readable medium before the apparatus receives any sequence data for the target nucleotide sequences.

In an additional embodiment, the invention relates to a method for detecting the presence of a genetic mutation that alters gene expression, comprising the steps of a) obtaining a plurality of target nucleotide sequences; b) aligning the target nucleotide sequences with a set of reference nucleotide sequences comprising a first reference sequence and at least one additional reference sequence; c) quantifying the number of target nucleotide sequences that align with each of the reference nucleotide sequences; and d) comparing the quantity of target nucleotide sequences that align with the first reference nucleotide sequence to the quantity of target nucleotide sequences that align with the other reference nucleotide sequences, wherein an increase or decrease in the quantity of target nucleotide sequences that align with the first reference nucleotide sequence relative to the quantity of target nucleotide sequences that align with the other reference nucleotide sequences is indicative of a genetic mutation that alters gene expression.

In one embodiment, the genetic mutation is a structural variation (e.g., rearrangement, deletion, insertion or repetition). Typically, a structural variation will involve about 50 to about 25,000 base pairs of DNA.

In another embodiment, the genetic mutation is a copy-number-variation (e.g., a copy-number-variation involving a rearrangement, deletion, insertion or repetition). Typically, a copy-number-variation will involve about 25,000 to about 250,000,000 base pairs of DNA.

Other examples of genetic mutations that alter gene expression include mutations (e.g., SNPs) that alter (e.g., increases, decreases) the expression of an RNA transcript.

In one embodiment, the target nucleotide sequences being analyzed are obtained from the products of one or more nucleic acid amplification reactions, such as, for example, a polymerase chain reaction (PCR) (e.g., a multiplex polymerase chain reaction, a single-plex polymerase chain reaction).

In another embodiment, the target nucleotide sequences being analyzed are obtained from the products of a restriction digest.

In yet another embodiment, the target nucleotide sequences being analyzed are obtained from the products of a reverse transcription (RT) reaction.

Typically, the target nucleotide sequences will be obtained with the aid of a sequencer instrument, such as, for example, a Next Generation Sequencer (NGS) sequencer.

The plurality of target nucleotide sequences that are being analyzed can include unaligned sequences, paired sequences or unpaired sequences, or a combination thereof.

In another embodiment, the invention relates to a method for detecting a genetic mutation, comprising the steps of a) amplifying three or more target nucleotide sequences in a sample comprising genomic DNA to produce an amplicon for each target nucleotide sequence; b) sequencing the amplicons; and c) analyzing the sequences of the amplicons for the presence of a genetic mutation. In some embodiments, the three or more target nucleotide sequences include a) at least one target nucleotide sequence is being analyzed for a single nucleotide polymorphism (SNP), b) at least one target nucleotide sequence is being analyzed for an insertion, a deletion, or an insertion and a deletion, and c) at least one target nucleotide sequence is being analyzed for a rearrangement.

Suitable nucleic acid amplification reactions for amplifying target nucleotide sequences are known in the art. In one embodiment, the amplifying is performed using a polymerase chain reaction (PCR). The PCR can be a multiplex PCR reaction, a singleplex PCR reaction, or a combination thereof. Preferably, the three or more target nucleotide sequences are amplified simultaneously in a single reaction vessel.

In a particular embodiment, the amplifying step comprises two successive amplification reactions, wherein the first amplification reaction produces a plurality of first amplicons comprising the target sequence and an adapter, and the second amplification reaction produces a plurality of second amplicons that further comprise an index sequence and a platform-specific sequence (e.g., a platform-specific sequence for massively parallel sequencing (MPS)). In general, the first amplification reaction is performed using a different pair of target-specific primers for each target nucleotide sequence, and at least one primer in each pair includes an adapter. Preferably, the adapter is added to the 5′ end of the target sequence in each first amplicon.

In some embodiments, the target-specific primers are designed to produce an amplification product only if a mutation (e.g., a rearrangement, such as an inversion, a translocation or a duplication) is present. For example, a PCR reaction can be performed on the nucleic acid template in order to produce a library of molecules of varying but expected sizes; included in the reaction are Dummy PCR primers that flank the border(s) of the genomic rearrangement (see FIGS. 3A and 3B). The Dummy Primers are designed such that in cases where the sample being tested is canonical for the mutation (thus the template nucleic acid does not contain the rearrangement) the primers hybridize in an orientation that is incompatible with viable PCR amplification (they hybridize to locations on different chromosomes or RNA transcript or if they do hybridize to the same template molecule that do so at a distance apart (greater than or about 10 kb) or in an orientation (positive strand vs. negative strand) that will not produce an amplification product after PCR (see FIG. 3C, top).

If the template nucleic acid does contain the rearrangement, the Dummy primers will result in an amplification product (see FIG. 3C, bottom). After PCR, this pool of amplicons is analyzed by massively parallel sequencing and the distribution of molecule sizes is determined by the length of the sequencer reads (or the overlap of sequencer reads in the case of paired-read sequencing.) Prior to alignment to any reference sequences, the reads are separated into bins based on the size (in contiguous bp) of the molecules in the sequencing library to which the reads correspond. The number of different bins, the exact size of each bin and the sequence content of the amplicons that occupy each bin are known for canonical samples that contain no indels or genomic rearrangements. Reads that fall into bins that are not expected and reads that fall into expected bins but do not match the sequence of bases that are expected for that expected bin are aligned to a unique set or hash of reference sequences. Every bin, expected or not, will have its own unique rearrangement hash of reference sequences that contain every variation of rearrangement that 1) could be produced using the particular set of dummy primers included in the reaction and 2) would result in amplicons that match the size of the bin.

Typically, the first amplicons will each have a size in the range of about 50 to about 450 base pairs. In one embodiment, the first amplicon for each target nucleotide sequence will differ in size from each of the other first amplicons (e.g., by at least two base pairs).

The method can further include the step of purifying the first amplicons prior to performing the second amplification reaction, if desired.

In some embodiments, the second amplification reaction is performed using pairs of sequencer-specific primers comprising an index sequence and a platform-specific sequence (e.g., for massively parallel sequencing (MPS)).

The sequences can be analyzed for the presence of a genetic mutation using, for example, any of the sequence analysis methods described herein. For example, the step of analyzing the sequences of the amplicons for the presence of a genetic mutation can include sorting the target nucleotide sequences into a plurality of bins according to size; assigning a unique set of reference nucleotide sequences to each bin, wherein the reference nucleotide sequences include non-canonical reference sequences; aligning the target nucleotide sequences in each bin with the set of reference nucleotide sequences assigned to the bin; quantifying the number of target nucleotide sequences in a bin that align with each non-canonical reference sequence. The presence of a genetic mutation in a target nucleotide sequence is indicated, for example, that aligns with a non-canonical reference sequence in a bin is indicative of a genetic mutation in the target nucleotide sequence.

In some embodiments, the genetic mutation is a mutation that is associated with cancer (e.g., one or more cancers). In one embodiment, the genetic mutation is associated with lung cancer (e.g., non-small cell lung carcinoma (NSCLC)). In another embodiment, the genetic mutation is associated with colorectal cancer. In an additional embodiment, the genetic mutation is associated with skin cancer (e.g., melanoma). In yet another embodiment, the genetic mutation is associated with leukemia (e.g., acute myeloid leukemia).

Examples of mutations that are associated with cancer include various SNPs in the human KRAS, BRAF, EGFR, and KIT genes, insertions or deletions (e.g., having a size in the range of about 3 to about 300 base pairs) in the human EGFR, ERBB2, and FLT3 genes, and rearrangements producing fusion of the human ELM4 gene (NCBI Reference Sequence: NM_019063.3) and human ALK gene (NCBI Reference Sequence: NM_004304.4). Other examples of mutations that are associated with cancer include rearrangements producing any of the fusions listed in Table 4.

TABLE 4
Exemplary gene fusions associated with cancer
Gene fusion

| | | |
|---|---|---|
| EML4-ALK | CHCHD7-PLAG1 | KIF5B-ALK |
| NPM1-ALK | HMGA2-FHIT | MSN-ALK |
| BCL2-IgH enhancer | | |
| c-MYC-IgH enhancer | | |
| BCL6 gene | | |
| translocations | | |
| TMPRSS2-ETS gene | | |
| family | | |
| EWS-ETS gene family | | |
| ETV6-NTRK3 | HMGA2-NFIB | MYH9-ALK |
| CCND1-IgH | CRTC1-MAML2 | RANBP2-ALK |
| CCND2-Ig loci | | |
| BCR-ABL | CRCT3-MAML2 | SEC31A-ALK |
| FIG/GOPC-ROS1 | EWSR1-POUF5F1 | SQSTM1-ALK |
| SLC343A2-ROS1 | TMPRSS2-ERG | TFG-ALK |
| CD74-ROS1 | TMPRSS2-ETV1 | TPM3-ALK |
| SDC4-ROS1 | TMPRSS2-ETV4 | TMP4-ALK |
| TPM3-ROS1 | TMPRSS2-ETV5, | MLL-AFF1( |
| EZR-ROS1 | HNRNPA2B1-ETV1 | MLL-AFF1 (AF4) |
| LRGI3-ROS1 | HERV-K-ETV1 | MLL-MLLT3 (AF9) |
| KDELR2-ROS1 | C15ORF21-ETV1 | MLL-MLLT1 (ENL) |
| CCDC6-ROS1 | SLC45A3-ETV1 | MLL-MLLT10 (AF10) |
| YWHAE-ROS1 | SLC45A3-ETV5 | MLL-MLLT4 (AF6) |
| TFG-ROS1 | SLC45A3-ELK4 | MLL-ELL |
| CEP85L-ROS1 | KLK2-ETV4 | MLL-EPS15 (AF1p) |
| KIF5B-RET | CANT1-ETV4 | MLL-MLLT6 (AF17) |
| CCDC6-RET | RET-PTC1/CCDC6 | MLL-SEPT6 |
| NCOA4-RET | RET-PTC2/PRKAR1A | MLL-EP300 (P300) |
| TRIM33-RET | RET-PTC3,4/NCOA4, | MLL-CREBBP(CBP) |
| BRD4-NUT | RET-PTC5/GOLGA5 | MLL-AFF3 (LAF4) |
| BRD3-NUT | RET-PTC6/TRIM24 | MLL-AFF4 (AF5q31) |
| KIAA1549-BRAF | RET-PTC7/TRIM33, | CALM-AF10 |
| BCAS4-BCAS3 | RET-PTC8/KTN1 | SET-NUP214 |
| TBL1XR1-RGS17 | RET-PTC9/RFG9 | (DEK-CAN)-NUP214 |
| ODZ4-NRG1 | RET-PCM1 | |
| MALAT1-TFEB | TFG-NTRK1, | |
| ASPSCR1-TFE3 | TPM3-NTRK1 | |
| PRCC-TFE3, | TPR-NTRK1 | |
| CLTC-TFE3 | RET-D10S170 | |
| NONO-TFE3 | ELKS-RET | |
| SFPQ-TFE3 | HOOKS3-Ret, | |
| EWSR1-ATF1 | RFP-RET | |
| MN1-ETV6 | AKAP9-BRAF | |
| CTNNB1-PLAG1 | PAX8-PPARG | |
| LIFR-PLAG1 | ATIC-ALK | |
| TCEA1-PLAG1 | CARS-ALK | |
| FGFR1-PLAG1 | CLTC-ALK | |

In yet another embodiment, the invention is a kit for detecting a genetic mutation, comprising a first probe set comprising target-specific primers and a second probe set comprising sequencer-specific primers. In some embodiments, the first probe set comprises a) a pair of target-specific primers for detecting a single nucleotide polymorphism (SNP) in at least one target nucleotide sequence, b) a pair of target-specific primers for detecting an insertion, a deletion, or an insertion and a deletion in at least one target nucleotide sequence, and c) a pair of target-specific primers for detecting a rearrangement in at least one target nucleotide sequence.

In one embodiment, at least one primer in each pair of target-specific primers includes an adapter. In an additional embodiment, the target-specific primers are designed to produce an amplicon only when a rearrangement is present.

In another embodiment, each pair of sequencer-specific primers includes at least one primer that comprises an index sequence and a platform-specific sequence for massively parallel sequencing (MPS).

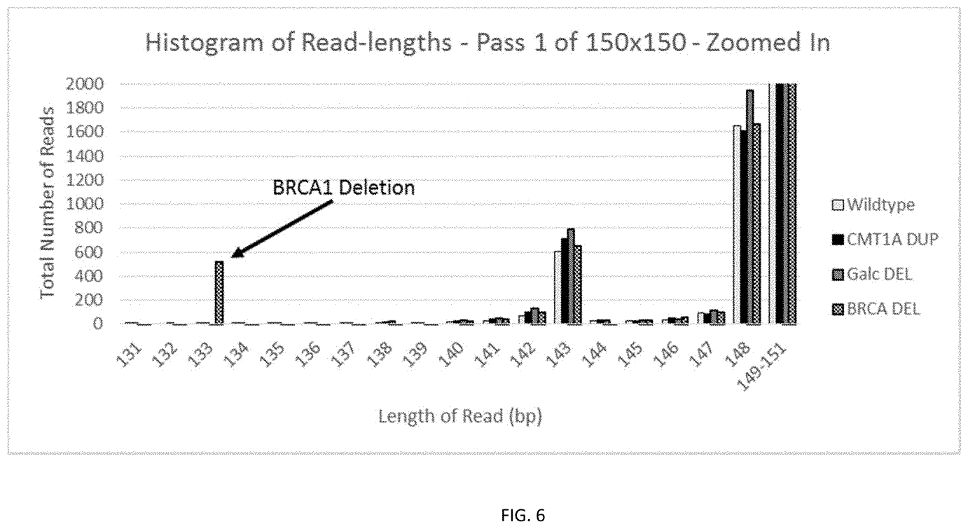

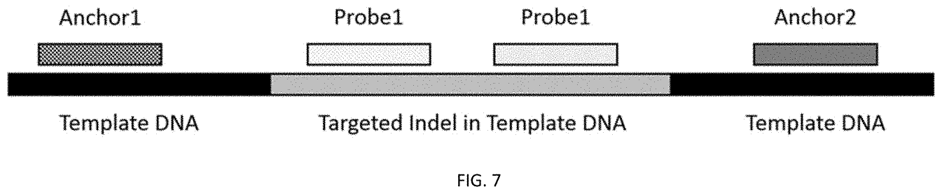

The kits described herein can include any single pair of primers, or any combination of primer pairs, such as primers listed in FIGS. 6 and 7.

In one embodiment, the first probe set comprises target-specific primers for a target nucleotide sequence that is present in a gene selected from the group consisting of human KRAS, human BRAF, human EGFR, and human KIT.

In another embodiment, the first probe set comprises target-specific primers for a target nucleotide sequence that is present in a gene selected from the group consisting of EGFR, ERBB2, and FLT3.

In another embodiment, the first probe set comprises target-specific primers for a target nucleotide sequence that is indicative of an ELM4-ALK fusion.

In some embodiments, the kits disclosed herein also comprise reagents for performing a DNA amplification reaction. In a particular embodiment, the reagents for performing a DNA amplification reaction are PCR reagents. PCR reagents include, for example, a DNA polymerase, an amplification buffer, and deoxynucleotides (dNTPs).

In another embodiment, the invention is a method of identifying a small mutation, which includes mutations affecting about five or fewer nucleotides of a nucleic acid molecule. Thus, a small mutation can affect about 1, 2, 3, 4, or 5 nucleotides in a nucleic acid. Nucleotides can be affected by an insertion, which includes duplications, deletion, translocation, or single-polynucleotide polymorphism (SNP).

In additional embodiments, methods of the invention can identify a medium mutation and/or a large mutation. Medium and large mutations can be defined by the read length (i.e., length of read) that a particular instrument can achieve. A medium mutation can include mutations that span about 5% to about 100% the length of read for a particular instrument or sequencing methodology. A medium mutation may have a length that corresponds to about 5%, 10%, 20%, 30%, 40%, 50%, 60%, 70%, 80%, 90%, or 100% of the read length of a sequencing instrument that is utilized in the method. A large mutation may include mutations that span more than about 100% the length of read for a particular instrument or sequencing methodology. In other embodiments there is no particular limitation the length of large mutations that can have, and the large mutation be of any size that is smaller than the nucleic acid being analyzed. Thus, in specific embodiments large mutations comprise mutations with a length that corresponds to about 200%, 300%, 400%, 500%, 600%, 700%, 800%, 900%, 1000%, or more of the read length of a sequencing instrument that is utilized in the method.

The generation of amplicons can be accomplished, for example, in a nucleic acid amplification reaction that uses nucleic acid primers (e.g., oligonucleotide primers). In general, a primer includes about 6 to about 100 (e.g., about 15 to about 40) contiguous nucleotides (e.g., deoxyribonucleotides, ribonucleotides). The contiguous nucleotides can be joined by covalent linkages, such as phosphorus linkages (e.g., phosphodiester, alkyl and aryl-phosphonate, phosphorothioate, phosphotriester bonds), and/or non-phosphorus linkages (e.g., peptide and/or sulfamate bonds). In some embodiments, one or more nucleotides in a primer can be modified. Exemplary modifications include, for example, methylation, substitution of one or more of the natural nucleotides (e.g., A, T, C, G, U) with a nucleotide analog, internucleotide modifications such as uncharged linkages (e.g., methyl phosphonates, phosphotriesters, phosphoamidates, carbamates, and the like), charged linkages (e.g., phosphorothioates, phosphorodithioates, and the like), pendent moieties (e.g., polypeptides), intercalators (e.g., acridine, psoralen, and the like), chelators, alkylators, and modified linkages (e.g., alpha anomeric nucleic acids, and the like). In a particular embodiment, a primer includes a locked nucleic acid (LNA).

The amplification of amplicons can be accomplished by any method known in the art, including polymerase chain reaction (PCR), reverse transcription reactions, or the like. The amplicon will generally have a read length that is less than or equal to the read length of a particular sequencing methodology. For example, if an ILLUMINA® NGS platform is employed, the read length is generally about 500 bases, and the amplicon will comprise about 500 or fewer bases. Alternatively, if an ION TORRENT™ NGS platform is utilized, the read length is generally about 100 bases, and the amplicon will comprise about 100 or fewer bases.

The amplicon can be an amplicon that is wholly contained within a region of the nucleic acid sequence that is being targeted. In other embodiments, the amplicon can also be partially contained within and/or can fall outside of a portion of a nucleic acid sequence that is known, suspected, or being tested for a mutation. In some embodiments, the method of the invention is configured to produce amplicons that are contained within a region of the nucleic acid sequence that is being targeted because it corresponds to a known mutation.

After the step of amplifying one or more regions within a nucleic acid molecule, the resulting amplicons then can be sequenced, counted, or both sequenced and counted. One of ordinary skill in the art would know the appropriate well-established methods, such as MPS, that can be utilized to sequence and/or count amplicons produced in the amplifying step. Sequencing the amplicons includes determining a nucleotide sequence of the amplicons that have been amplified in the amplifying step. Counting includes counting the number of each of different amplicons that have been amplified. In some embodiments counting can also refer to calculating a ratio of the number of first amplicons (e.g., a probe amplicon) to a number of a second amplicons (e.g., an anchor amplicon) in a sample.

In this respect, the methods described herein can identify small mutations, medium mutations, or a combination thereof in a particular sequence. In some embodiments, after the steps of amplifying and sequencing the amplicons, the sequence of a particular amplicon can be determined. Then, the sequence of the amplicon can then be aligned with a portion of a reference sequence. Those of ordinary skill would know the appropriate well-established methods and systems suitable for aligning certain amplicons to a portion of a reference sequence. In some embodiments the amplicon in the amplifying step is a “probe amplicon,” or an amplicon that wholly or partially overlaps a target sequence that is known, suspected, or being tested for a mutation. Thus, once the probe amplicon has been amplified, sequenced, and aligned with a portion of a reference sequence, the sequence of the probe amplicon can be compared to the sequence of the reference nucleic acid molecule. Comparison of the probe amplicon to the reference amplicon will show whether the tested nucleic acid molecule, and specifically the portion of the nucleic acid molecule that has been amplified, contains any nucleotide substitution, insertions, or deletions when compared to the reference sequence. In some embodiments, the method of comparing the sequence of a probe amplicon to the reference sequence can identify one or more single-nucleotide polymorphisms (SNPs) in a nucleic acid molecule. If the amplicon contains any such variations with respect to the reference sequence, the target sequence of the tested nucleic acid molecule can be identified as comprising a mutation (i.e., the target mutation).

Mutations, including small mutations and medium mutations, can also be identified by comparing the length of a particular amplicon to the expected length of that amplicon. The expected length of the amplicon corresponds to the length of the amplicon when obtained from a reference sequence. In some embodiments the target sequence can be identified as including one or more deleted nucleotides if a probe amplicon has a shorter length than if the probe amplicon had been obtained from a reference sequence. On the other hand, in some embodiments the target sequence can be identified as including one or more inserted nucleotides if a probe amplicon has a longer length than if the probe amplicon had been obtained from a reference sequence.

The methods described herein can also be utilized to identify medium mutations, large mutations, or a combination thereof in a nucleic acid molecule. In some embodiments the amplifying step of the method include selecting one or more “probe amplicons” to be amplified and one or more “anchor amplicons” to be amplified. As described above, the probe amplicon will be wholly or partially within a target sequence, or a portion of the nucleic acid sequence that is known, suspected, or being tested for a mutation. The anchor amplicon refers to an amplicon of a portion of the sequence of the nucleic acid molecule that is known or suspected to be free from any mutation, or at least the mutation being targeted. In specific embodiments, the anchor amplicon is a portion of the nucleic acid molecule that is relatively close to and flanks an end of a target sequence.

In some embodiments, the sequence of the anchor amplicon and the sequence of the probe amplicon are selected to by sequences that are known to amplify and transcribe at substantially equal rates. In other embodiments, the sequence of the anchor amplicon and the sequence of the probe amplicon amplify at different rates, but the difference in amplification rate is known. In this respect, and as discussed further below, further steps in the present methods can comprise identifying differences in the presence and concentration of the anchor amplicons and probe amplicons. Thus, if they have substantially equal amplification rates, the ratio of anchor amplicons to probe amplicons after the amplification step should correspond to the ratio of the sequence for the anchor amplicon to the sequence for the probe amplicon in the nucleic acid being analyzed. If the amplification rates are not equal the final ratio may not be indicative of the proportion of these sequences in the nucleic acid molecule. However, if the difference in amplification rate is known, in some methods one can account for certain disparities in the concentration of anchor amplicons and probe amplicons.

After the amplification step is performed, the number of each probe amplicon and the number of each anchor amplicon is counted. One of ordinary skill in the art would know suitable, well-established methods for counting the number amplicons, including MPS. The ratio of the anchor amplicons to the probe amplicons, or vice versa, can also be calculated. The numbers and/or ratios of the anchor amplicons to the probe amplicons will indicate whether the number of probe amplicons is lower than, approximately equal to, or greater than the number of anchor amplicons.

The method includes identifying the presence or absence of the nucleic acid molecule is a target mutation by determining whether there are discrepancies between the numbers or ratios of the probe amplicons and the number of anchor amplicons. A relatively lower number of probe amplicons in comparison to anchor amplicons generally indicates that at least the portion of the reference sequence that corresponds to the probe amplicon is absent to some degree from the nucleic acid molecule. In some embodiments this indicates that the nucleic acid molecule is at least partially lacking a target sequence or a portion of the target sequence. Thus, a nucleic acid molecule can be identified as including a deletion if the number of probe amplicons is lower than a number of anchor amplicons. Other embodiments a nucleic acid molecule can be identified as includes an insertion if the number of probe amplicons is higher than a number of anchor amplicons.

A similar determination can be made by determining the ratio of a probe amplicon to anchor amplicons. For example, a ratio of probe amplicon to anchor amplicon that is greater than about 1:1 can be used to identify the nucleic acid molecule as comprising an insertion, whereas a ratio of probe amplicon to anchor amplicon that is less than about 1:1 can be used to identify the nucleic acid molecule as comprising a deletion.

In this regard, the methods described herein can be utilized to identify large mutations; that is, mutations that are longer than the read length of a particular sequencing method. For instance, the probe amplicon may be an amplicon that is within, but shorter in length than, the length of a target sequence. If the present methods indicate that the probe amplicons, which should be within the target sequence, is present at a lower concentration than the anchor amplicons, then the method can identify that the entire target sequence as being deleted. That is, the probe amplicon can identify a target mutation that is greater in length than the probe amplicon, the read length being utilized, or both. The method described herein can identify mutations, including deletions and/or insertions, that are larger than a read length offered by a standard sequencing method.

The methods of the invention can further be employed to identify whether particular mutations are homozygous or heterozygous. In some embodiments, a homozygous mutation provides for two copies of a gene that includes a target mutation. On the other hand, a heterozygous mutation causes the nucleic acid molecule to include one gene that includes the target mutation and one gene that does not include the target mutant. After the amplifying step, a mutation that is homozygous can show a larger disparity between the concentration of anchor amplicons and probe amplicons when compared to a mutation that is heterozygous. Therefore, in some embodiments, a relatively larger difference between the number of anchor amplicons and the number probe amplicons can indicate that the mutation (i.e., insertion or deletion) is homozygous, whereas a relatively smaller difference between the number of anchor amplicons and the number of probe amplicons can indicate that the mutation is heterozygous.

In some embodiments, a plurality of anchor amplicons, a plurality of probe amplicons, or both a plurality of anchor amplicons and a plurality of probe amplicons are utilized to identify target mutations. In specific embodiments, one anchor amplicon can be compared to two or more of the plurality of probe amplicons and/or one probe amplicon can be compared to two or more of the plurality of anchor amplicons. Use of two or more anchor and/or probe amplicons can average the counts of the amplicons and can reduce or eliminate the incidences of false positives. Such embodiments can also increase the sensitivity with which the present methods can identify a mutation in a nucleic acid molecule.

The methods described herein may also be utilized to identify small mutations, medium mutations, large mutations, or a combination thereof in a nucleic acid molecule. In some embodiments, the present methods can identify small and medium mutations, including particular SNPs, in a nucleic acid molecule while also identifying medium and large indels, including indels that may be longer than the read length of a particular sequencing method.

In an additional embodiment, the present invention is a method for identifying a target mutation in a nucleic acid molecule, comprising the steps of: amplifying an anchor amplicon and a probe amplicon in the nucleic acid molecule; counting the number of anchor amplicons and the number of probe amplicons; and identifying the nucleic acid molecule as comprising the target mutation if there is a statistically significant difference between the number of anchor amplicons and the number of probe amplicons.

The amplifying step of the method can include, for example, a multiplex PCR reaction, a Reverse Transcription (RT) reaction, or a combination thereof. The counting step of the method can include massively parallel sequencing (MPS). In another embodiment, the counting step includes determining the number of sequence reads from the nucleic acid molecule that align with the anchor amplicon, the probe amplicon, or a combination thereof. The alignment of the sequence reads is performed with MPS.

The identifying step of the method can include, for example, determining whether there is a statistically significant difference between the number of the anchor amplicons and the number of the probe amplicons for the nucleic acid molecule compared to a theoretical number of anchor amplicons and probe amplicons in a canonical nucleic acid molecule, or determining whether there is a statistically significant difference between a length of the probe amplicon and a length of a portion of a canonical version of the nucleic acid molecule that corresponds to the probe amplicon. A deletion is identified, for example, when there is a statistically significant lower number of the probe amplicons compared to the number of anchor amplicons, or when the length of the probe amplicon is less than the length of the portion of the canonical nucleic acid molecule that corresponds to the probe amplicon. An insertion is identified, for example, when there is a statistically significant higher number of the probe amplicons compared to the number of anchor amplicons, or when the length of the probe amplicon is greater than the length of the portion of the canonical version of the nucleic acid molecule that corresponds to the probe amplicon.

In some embodiments, the probe amplicon is wholly or partially contained within the target mutation.

The method described herein can further include sequencing a sequence of the probe amplicons; aligning a sequence of the probe amplicons to a sequence of a canonical sequence of the nucleic acid molecule; and identifying the nucleic molecule as comprising the target mutation if there is a difference between the sequence of the probe amplicons and the sequence of a canonical sequence of the nucleic acid molecule.

Examples of target mutations include a small mutation (e.g., SNP), a medium mutation (e.g., indel), a large mutation (e.g., rearrangement), or a combination thereof. The target mutation can also be a mutation that is associated with a disease or condition, such as, for example, a mutation associated with cancer. When the target mutation is associated with a disease or condition, the step of identifying a target mutation can include, for example, an additional step of diagnosing the nucleic acid molecule as being from a subject having and/or being at risk for developing the disease or condition.

In another embodiment, the invention is a system for performing a method for identifying a target mutation in a nucleic acid molecule, wherein the method includes amplifying an anchor amplicon and a probe amplicon in the nucleic acid molecule; counting the number of anchor amplicons and the number of probe amplicons; and identifying the nucleic acid molecule as comprising the target mutation if there is a statistically significant difference between the number of anchor amplicons and the number of probe amplicons.

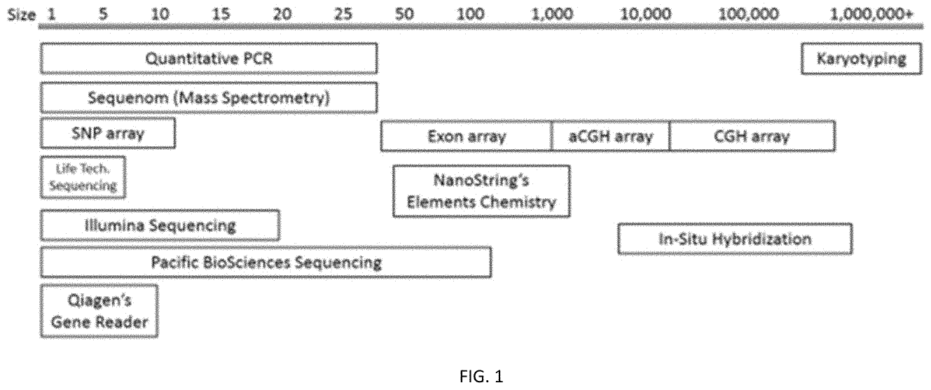

Currently, mutation detection technologies are limited in the size of mutation that can be detected, i.e. either detect small mutations (about 1 to about 20 bases), medium-sized mutations (about 21 to about 150 bases) or large mutations (greater than about 150 bases), but not all three (see FIG. 1). The invention disclosed herein is useful for detecting small, medium and large mutations.

Genetic mutations can affect many of the biological processes that are related to human disease. Thus, their detection and characterization is critical to several fields of research as well as in a broadening range of medical fields. In medicine, genetic tests are generally performed for several reasons. First, to either confirm or rule out the possibility that a patient has inherited a genetic disorder. In these cases the patient has demonstrated symptoms that have been linked to mutations in a particular gene or routine laboratory screenings have shown atypical results. The physician that orders the test uses it as a diagnostic tool to identify the root cause of their patient's problems and the results allow the physician to move forward with treatment. A second reason for performing genetic tests is to determine whether or not a person is a carrier of certain genetic variants. This generally occurs after a family member has been diagnosed with an inherited disorder. The results can be used for family planning, such as in determining whether parents carry the Cystic Fibrosis gene, or in taking preventative measures to preserve health, such as with the BRCA genes that have been linked to breast cancer (e.g., heritable breast cancer).

A third application of genetic testing is to enable physicians to tailor a patient's therapy to match their genetic makeup. This phenomenon is commonly referred to as “Personalized Medicine” and has become a key part of most pharmaceutical companies' development strategies(15). A study by the Tufts Center for the Study of Drug Development found that pharma spending on Personalized Medicine R&D more than doubled from 2003-2009, a trend that is expected to continue over the coming decades. For example, a potential benefit of Personalized Medicine, such as XALKORI® anti-cancer drug. Released in August 2011, this compound is highly targeted and extremely effective, but only in the about 5% of lung cancer patients whose tumors are driven by a mutation involving the ALK gene. For patients with this specific mutation XALKORI® anti-cancer drug is a miracle drug, for those who lack the mutation it is a waste of time and money. In order to prescribe XALKORI® anti-cancer drug a physician must determine a patient's ALK status using a genetic test, in this context the test is referred to as a Companion Diagnostic (CDx)(16). There are hundreds of targeted drugs like XALKORI® anti-cancer drug currently in clinical trials with hundreds more on the way. This represents a huge opportunity for a genetic testing laboratory because each one of these therapies will require a CDx test to identify the patients that will respond favorably to the drug.

Similarly, counting genetic or epigenetic changes in tumors can inform fundamental issues in cancer biology(17). Mutations are a significant component of current problems in managing patients with viral diseases, such as AIDS and hepatitis, by virtue of the drug-resistance that can occur(18),(19). Detection of such mutations, particularly at a stage, prior to mutations emerging as dominant in the population, will likely be essential to the optimization of therapy. Detection of donor DNA in the blood of organ transplant patients is an important indicator of graft rejection and detection of fetal DNA in maternal plasma can be used for prenatal diagnosis in a noninvasive fashion (20), (21). In neoplastic diseases, which are related to somatic mutations, the application of rare mutant detection is critical; and can be used to help identify residual disease at surgical margins or in lymph nodes, to follow the course of therapy when assessed in plasma, and perhaps to identify patients with early, surgically curable disease when evaluated in stool, sputum, plasma, and other bodily fluids(22), (23), (24). These examples highlight the importance of identifying rare mutations for both basic and clinical research as well as modern medical practice. Accordingly, innovative ways to assess them have been devised over the years.

A genetic test can be any laboratory procedure to identify or detect changes in the sequence of chemical bases that makeup an individual's DNA. There are numerous methods for detecting mutations; most infer their presence indirectly by analyzing changes in the DNA's ability to bind primers (small fragments of DNA that complement sections of a gene) or measuring alterations in proteins rather than changes in the DNA itself. While most genetic disorders can be caused by numerous different mutations, most genetic tests can only detect a few mutations at a time. Tests are also limited the size of mutation they can detect. Mutations range in size from a change in a single base-pair (bp) up the complete removal of an entire chromosome comprising hundreds of millions of bp. Every technology can vary in the mutations that can be detected and lack in spanning the whole range, as described below. A limitation of existing technologies is that in order for a lab to provide viable genetic tests, several costly instruments must be purchased and maintained by technical staff.

Exemplary commonly techniques used to perform genetic tests include the following:

Quantitative PCR (gPCR)—

This technique is relatively inexpensive and can provide information quickly (<2 days.) Results are quantitative and simple to interpret. Limitations of qPCR assays are the limited ability to generally detect only a single mutation at a time, must be designed for identifying a specific mutation in mind and, thus, cannot detect unknown variants.

Arrays—

Also referred to as microarrays, arrays have the advantage of simultaneously detecting numerous simple mutations. Disadvantages include high-cost, low sensitivity, a tendency to pick up background noise and an inability to detect unknown mutations.

In-Situ Hybridization (ISH)—

This technique is moderately in-expensive and sensitive but only suited for detecting large scale mutations that involve large chunks of DNA. Interpretation is difficult and requires a specially trained pathologist. Accuracy is limited by the qualitative nature of the readout. Results are often ambiguous and unusable. Also called FISH when fluorescently labeled probes are used.

Immunohistochemistry (IHC)—

This technique uses the specificity of antibody-protein interactions to detect mutant proteins in cells. A limitation is detection of the secondary effect of genetic mutations rather than the presence of the mutations themselves.

Massively parallel sequencing represents a particularly powerful genetic testing tool in which hundreds of millions of template molecules can be analyzed one-by-one. An advantage of IHC over conventional methods is the comprehensiveness, covering numerous potential mutations simultaneously and in an automated fashion. The drawback of massively parallel sequencing is that it lacks the sensitivity of qPCR and cannot generally be used to detect rare variants due to the high error rate associated with the sequencing process. For example, with the commonly used Illumina sequencing instruments, this error rate varies from about 1% (25), (26) to −0.05% (27), (28), depending on factors, such as the read length (29), use of improved base calling algorithms (30), (31), (32) and the type of variants detected(33). Some of these errors presumably result from mutations introduced during template preparation, during the pre-amplification steps required for library preparation and during further solid-phase amplification on the instrument itself. Other errors are due to base mis-incorporation during sequencing and base-calling errors. Advances in base-calling can enhance confidence (e.g., (18-21)), but instrument-based errors are still limiting, particularly in clinical samples wherein the mutation prevalence can be 0.01% or less(10). In the methods described herein, sequencing reactions are designed such that different populations of molecules in the sequencing library occupy known bins based on their size allows for sequences reads to be sorted prior to alignment. Since the identity and, thus, sequence content of the molecules expected to fall into each bin are already known, this pre-sorting allows reads to be aligned directly to a predetermined and finite set of reference libraries and produces genotyping data that can be more reliably interpreted, so that relatively rare mutations or difficult mutation types can be identified with commercially available instruments.

The methods, systems and kits described herein have improved sensitivity and accuracy of sequence determinations for investigative, clinical, forensic, and genealogical purposes.

### EXEMPLIFICATION

### Example 1

This Example demonstrates that methods described herein can detect, independently or simultaneously, a spectrum of mutations ranging in size. Such mutations range from SNPs affecting one base pair (bp) to a chromosomal rearrangement affecting portions of nucleic acid sequence millions of bases long.

An amplification step include a reaction in a single tube for approximately four hours was performed while processing 4 samples at a time. The samples were prepared for sequencing, and then sequenced on a MISEQ® desktop DNA sequencer (Illumina, San Diego, Calif.) using 150×150 cycling chemistry. The assay was designed to detect 5 different mutations, including: (1) a SNP in the MPZ gene, (2) a series of small deletions in BRCA1 exon 11 that are less than four bp long, (3) a 40 bp, Category I deletion found in BRCA1 exon 11, (4) a 30 kilo-base (kb), Category II deletion in the GALC gene, and (5) a 1.6 mega-base (Mb) Category II insertion that results in the duplication of the PMP22 gene.

Category I Indels include, for example, an insertion, deletion or combination of an insertion and a deletion involving of a section of DNA that is short enough to be detected by deviations from the expected amplicon size. Category I mutations fit within an amplicon without altering its size to the point that the amplicon is either too long to amplify, in the case or insertions, or too small to make it through the purification process that proceeds sequencing, in the case of deletions. An example of a Category I Indel is the 40 bp BRCA1 deletion discussed herein. This mutation alters this size of an amplicon expected to be about 173 base-pairs (bp) long, producing an amplicon that is 133 bp in size.

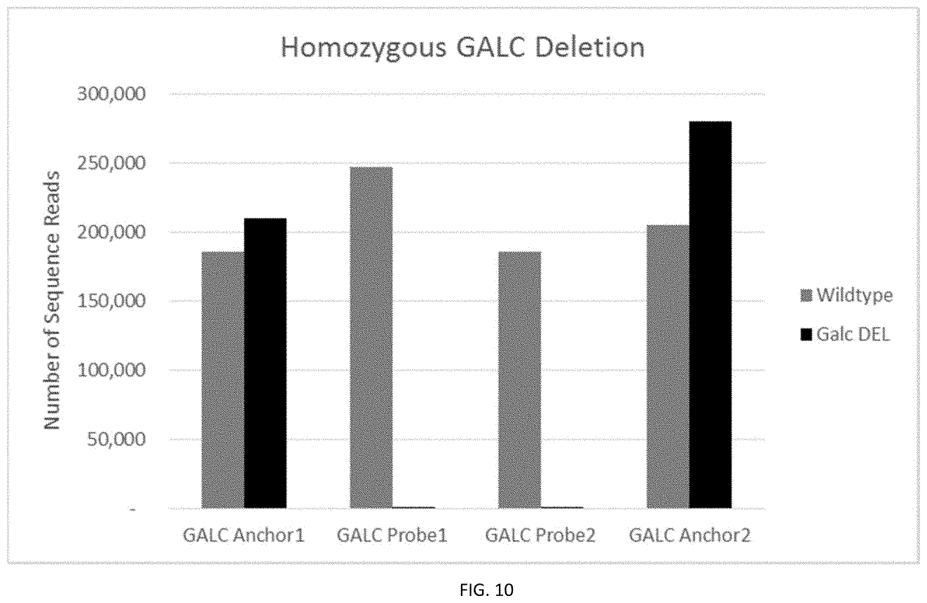

Category II Indels include, for example, an insertion, deletion or combination of an insertion and a deletion involving of a section of DNA that is too large to be amplified by PCR. These mutations cannot fit into amplicons and, therefore, cannot be detected by deviations from expected amplicon size. Instead these mutations are detected by deviations in the ratio of the number of Probe amplicons (amplification products generated from within the region of DNA suspected to be inserted or deleted) sequenced to the number of Anchor amplicons (amplification products generated from outside the region of DNA suspected to be inserted or deleted) sequenced. An example of a Category II Indel is the 30,000 bp GALC deletion discussed herein. To detect it, four amplicons were designed; 2 Probe amplicons that fall within the deleted region and 2 Anchor amplicons that fall outside of it. In samples that lack the deletion, all four amplicons are found in the resulting sequencing data. In a sample that is homozygous for the deletion, the two Anchor amplicons are present but the Probe amplicons are missing, see FIG. 10.

Four samples were analyzed. The samples were of human genomic DNA, and included: (1) a canonical reference sequence that contained none of the mutations listed above, (2) a BRCA deletion sequence that was heterozygous for 40 bp deletion in exon 11, (3) a GALC deletion sequence that was homozygous for 30 kb GALC deletion and was heterozygous for MPZ SNP, and (4) a CMT1A duplication sequence that was heterozygous for 1.6 Mb CMT1A insertion and heterozygous for MPZ SNP.

In the method, each reaction was a multiplex PCR that amplified a known set of amplicons. Each amplicon had a unique size at least 2 bp different from every other amplicon in the reaction because the DNA sequencer could measure the length of amplicons with a resolution of up to ±1 base. Specifically, the reaction amplified 10 different amplicons ranging in size from 143 bp to 176 bp.

TABLE 5
Size of amplicon relative to target mutations.

| Mutation target | Amplicon size (bp) |
|---|---|
| SNP - rs6674383 (MPZ) gene | 176 |
| BRCA1 Exon 11 indels | 173 |
| Upstream of GALC Deletion | 151 |
| GALC Deletion Region1 | 153 |
| GALC Deletion Region2 | 157 |
| Downstream of GALC Deletion | 143 |
| Upstream of CMT1A duplication | 169 |
| Region 1 of CMT1A duplication | 166 |
| Region 2 of CMT1A duplication | 162 |
| Downstream of CMT1A duplication | 148 |

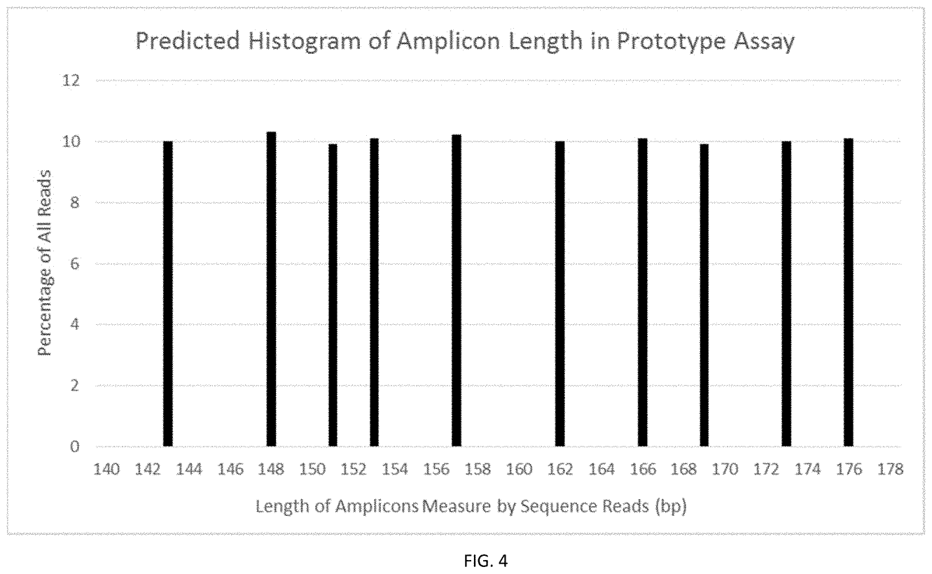

The histogram in FIG. 4 shows the expected distribution of amplicon read-length for the prototype assay described in the Table 5.

In order to the detect Category I indels, PCR primers were designed to flank the genetic regions where the indel occurred. In the amplifying step, the amplification primers produced double-stranded amplicons that would contained the indel if it was present in the template DNA sample.

The particular mutations that were identified included a series of deletions that are often found in exon 11 of the BRCA1 gene and can cause an increased risk of breast cancer. One of the four human samples was from a patient that was heterozygous for a 40 bp deletion in exon 11. One of the amplicons in the assay spanned the region where this deletion occurs. In the canonical samples, where the deletion was not present, the resulting BRCA1 amplicon was 173 bp long. In samples that contained the 40 bp deletion, the resulting BRCA1 amplicon was 133 bp long.

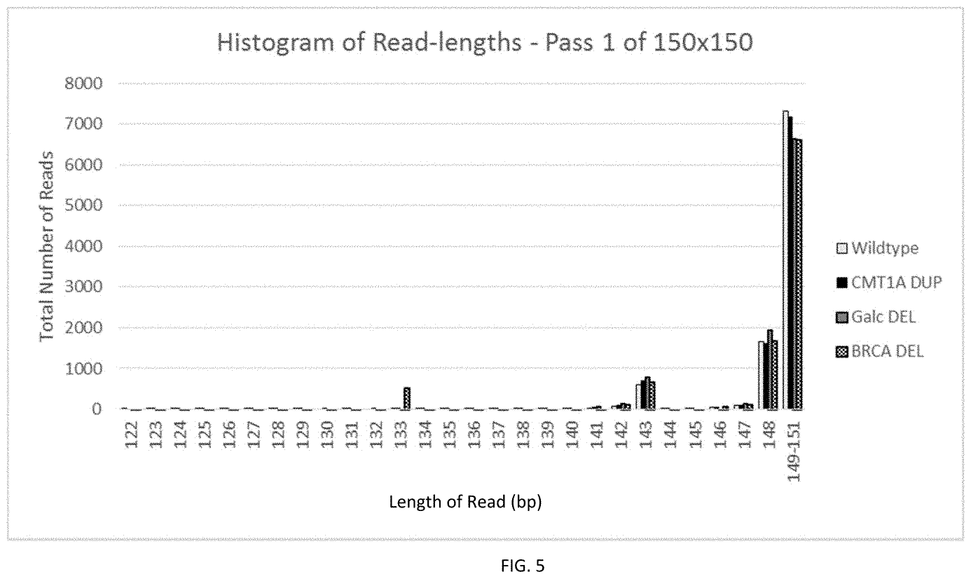

The histograms in FIGS. 5 and 6 show the amplicon size distribution from the first pass of a 150×150 paired-end run on a MISEQ® desktop DNA sequencer (Illumina, San Diego, Calif.). For the sake of computational efficiency, only the first 10,000 reads were analyzed, rather than the about 1.5 million reads produced by the sequencer. Each amplicon had gone through 150 cycles of single base additions, and thus all amplicons that were greater than 150 bp long should have produced sequence reads of 149, 150, or 151 bp.

In the pool most of the amplicons were greater than 150. Two of the amplicons were less than 150, and each of these showed up in all four samples at 148 bp and 143 bp. In the sample that contained the 40 bp deletion a peak showed up at 133 bp that was not present in the others. In the 10,000 reads from this sample, there were 548 that fell either at 133 or +1 bp therefrom. Of the 548 reads, 533 (97.3%) aligned to the sequence produced by the deletion, confirming the presence of the mutation.

Most of the reads maxed out at more than 149 bp, but two peaks were present in all four samples at the length of read of 143 bp and 148 bp (see FIGS. 5 and 6). In one sample there was a peak at 133 bp, which is 40 bp shorter than the 173 bp fragment that spans BRCA1 exonl 1. This outlier peak occurred in the sample that was heterozygous for a 40 bp deletion in BRCA1 exonl 1.

Medium sized indels, such as the 40 bp BRCA1 deletion described above are not uncommon in clinical genetics. The BRCA1 deletion is highly correlated with hereditary breast cancer. Another example is the FLT3 gene, which can contain numerous SNPS in its two kinase domains as well as insertions in Exons 13 and 14 that have been linked to patient prognosis in certain types of leukemia. The insertions are highly variable in size, ranging from 3-300 bp, with longer insertions linked to a poorer outcome for the patient. These insertions also tend to exact repetitions of sequence found in other parts of the FLT3 gene. Often 10-133 bp regions of exon 14 are in inserted into exon 13 and vice versa; they can also be tandemly repeated to make even larger insertions. This wide range of insertions could be detected in same the manner that the BRCA1 deletion was detected as described above; due to the fact the sequence inserted into FLT3 is most often a duplication of sequence that exists in other regions of the gene these indels can also be detected by the inclusion of a dummy primer. The dummy primer is located within the duplicated region; the reaction is designed such that canonical samples either produces no amplicon, because the primer orientation is incompatible with PCR, or produces an amplicon that is much larger (>2×) than the rest of the amplicons produced by the reaction. The larger amplicon will be outcompeted by the smaller ones and will eventually be drowned out and unlikely to interfere with the rest of the reaction. In instances where a duplication is present the dummy primer will produce an amplicon in the range of the other in the pool and be detectable by both variations in the expected amplicon length distribution and by sequence alignment. In the case of FLT3, the assay could be split into two reactions; one to detect insertions in exon 13 along with SNPs in some of the exons that comprise the kinase domain and one to detect insertions in exon14 and still more SNPs in other FLT3 exons. The reaction for exon 13 insertions would contain a dummy primer that lies in the region of exon14 that is often inserted into exon 13. In canonical samples, this produces an amplicon several hundred bases longer than the others in the pool; in samples with an insertion the “dummy” primer lies in such a way as to produce an amplicon of viable length that can detected by MPS. This information can be used to further support variations in the expected amplicon distribution as evidence that an insertion is present and make the assay more reliable and accurate.

### Example 2

This Example describes a method similar to that described in Example 1, which was used for identifying large mutations in a nucleic acid sequence. The large indels were larger than the read-length of the sequencer. However, to detect the indels, the quantitative nature of PCR was utilized to infer the presence of extra or missing chunks of DNA. Specifically, two different types of amplicons were identified; anchor amplicons that fell outside of the indel and probe amplicons that fell within the indel (see FIG. 7).

Samples that contained insertions should comprise more initial DNA template for the probe amplicons to amplify off of, which should result in a relatively greater amount of probe amplicons in the mix after PCR. In samples that contain large deletions, there should be less initial DNA template for the probe amplicons to amplify off of, which should result in a lower amount of probe amplicons in the mix after PCR. In both cases there should be a consistent amount of initial DNA template for the anchor probes to amplify off of, which should result in a consistent amount of anchor amplicons in the mix after PCR. This amount can be used as a reference standard to compare to the amount of probe amplicons present. Large indels were then detected by comparing the number of sequence reads corresponding to probes amplicons to the number of sequence reads corresponding to anchor amplicons. In samples that contained insertion, the ratio of probe amplicons to anchor amplicons should increase. In samples with deletions the ratio of probe amplicons to anchor amplicons should decrease.

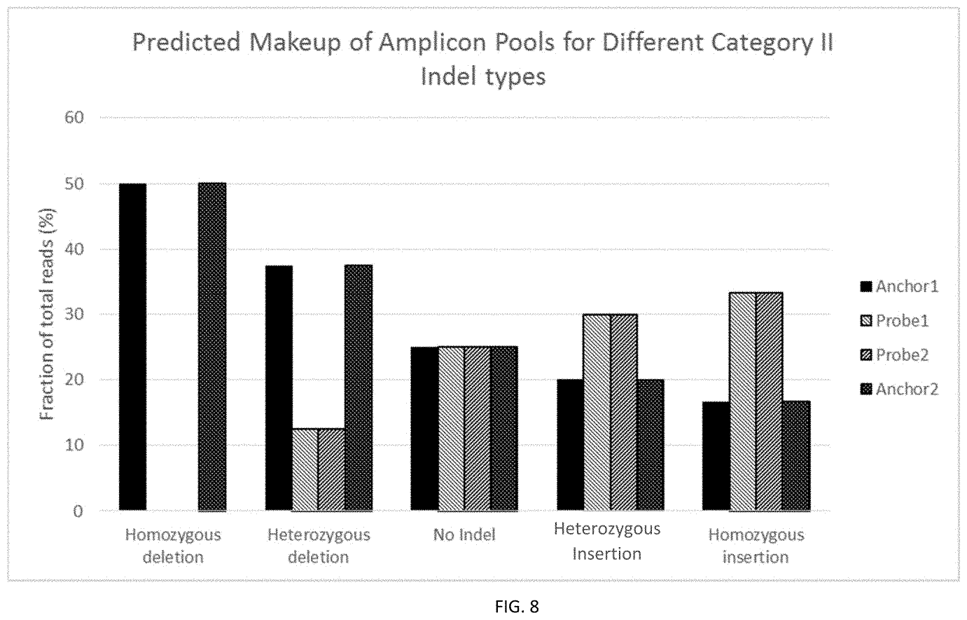

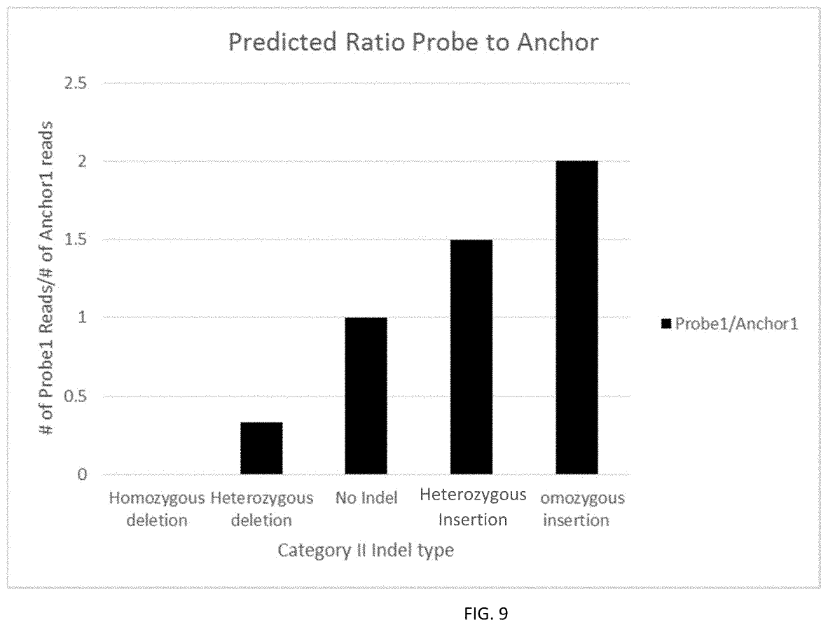

FIG. 8 is a graph showing what the pool of amplicons is predicted to look like after amplification in the schematic shown in FIG. 7. This effect can also be measured by comparing the ratio of the number of probe amplicons to the number anchor amplicons (see FIG. 9). FIGS. 8 and 9 illustrate how homozygous deletions, heterozygous deletions, no indel, heterozygous insertions, and homozygous insertions are predicted to affect the number, fraction, and ratios of probe amplicons and anchor amplicons. However, in a real setting, because each amplicon amplifies with a slightly different efficiency, the ratio in the canonical sample would not necessarily be exactly 1:1. This is not problematic, however, as the ratios do not all need to be the same, but they merely need to be consistent canonical so that any deviations caused by indels can be identified as being statistically significant from the range of values produced by canonical samples. Each method can be performed on a set of canonical samples to establish the normal range and put a cutoff value for calling mutations.

Two samples were run with the prototype assay from patients with Category II indels. One sample contained a homozygous deletion of 30 kb that resulted in the loss of exons 11-17 of the GALC gene which manifests as Krabbe disease, a rare but severe neurological disorder. The other contained a heterozygous duplication of 1.6 Mb of chromosome 17 called the CMT1A mutation that produces a third copy of the gene PMP22 and is one of the primary causes of Charcot-Marie-Tooth disease, a disorder that causes muscular degeneration.

The plot in FIG. 10 shows the distribution of reads for a canonical sample and a sample homozygous for the GALC deletion. The lack of reads within the indel region is evident by the lack of probe sequence reads.

Table 6 and two plots below compare the sample with the CMT1A duplication to canonical. This embodiment of the method involved a large number of processing steps after the initial amplification step, which can normalize the sample and mute differences between the amount of anchor amplicons and probe amplicons. The general trend showed that the ratio of probe amplicons to anchor amplicons was generally higher in the sample with the duplication.

TABLE 6
Ratio of probe amplicons to anchor amplicons.

| Ratios | Wildtype | CMT1A DUP | % change |
|---|---|---|---|
| Probe1/Anchor1 | 1.45 | 1.51 | 4% |
| Probe1/Anchor2 | 3.13 | 3.47 | 11% |
| Probe2/Anchor1 | 1.28 | 1.27 | −1% |
| Probe2/Anchor2 | 2.75 | 2.91 | 6% |

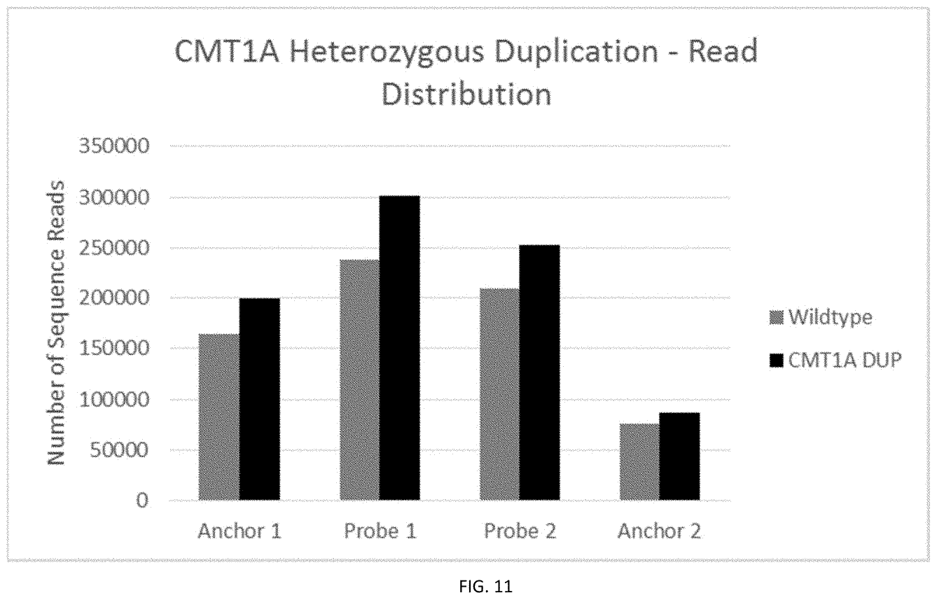

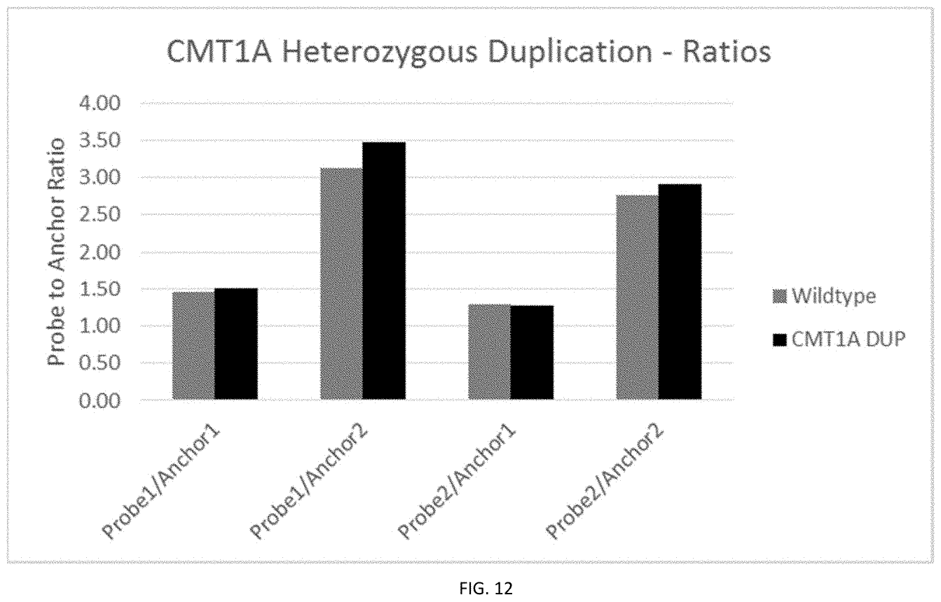

This presence of the duplication was less apparent than in the homozygous GALC deletion above where the probe amplicons were not present. In the sample with the duplication, the probe amplicons were slightly more prevalent than the anchor amplicons in the sample (see FIGS. 11 and 12).

Two of the samples also contained SNPs in the MPZ gene. These were identified using MPS analysis tools. Specifically, the two middle samples were heterozygous for a G to A switch.

### Example 3: Detection of Small, Medium and Large Mutations Associated with Cancer

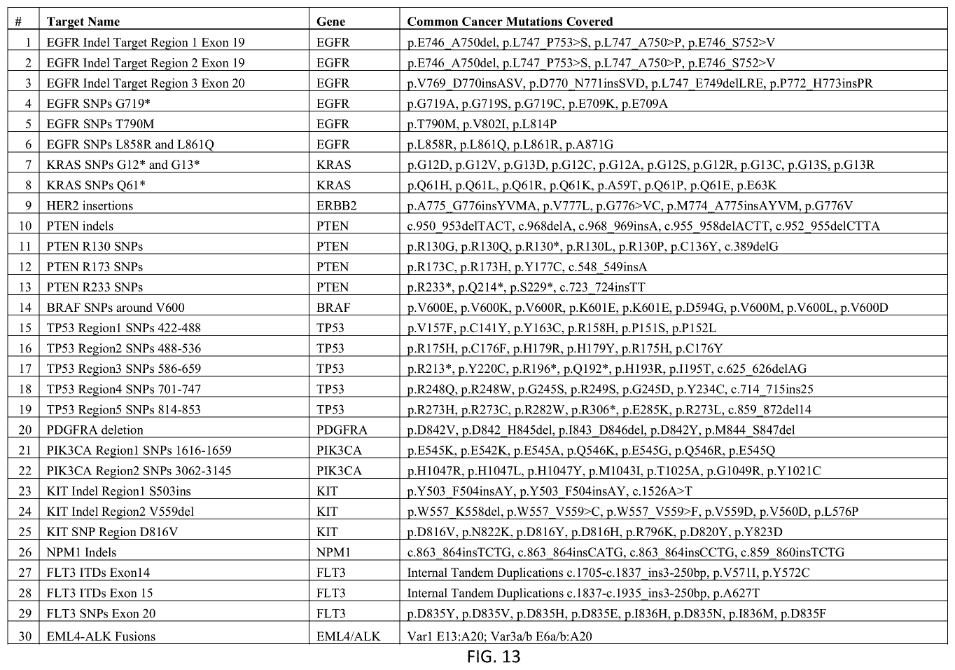

In this Example, a single test/assay was developed to demonstrate the ability of the present invention to detect small (KRAS, BRAF, EGFR and KIT SNPs), medium (EGFR Deletions, ERBB2 Insertions and FLT3 Internal Tandem Duplications) and large (EML4-ALK Inversions) mutations at low-levels within the same reaction. A wide-range of previously-characterized somatic mutations in cancer was detected. The mutations covered by the test tend to be of particular importance in therapeutic decision-making or have been correlated to patient prognosis. FIG. 13 provides a summary of the genetic regions targeted by this assay and the most common mutations found in each target.

The mutations assayed for in the test are described further below.

Small Mutations (1-2 base pairs in size)

KRAS—

Single Nucleotide Polymorphisms (SNPs) that result in single amino acid changes at either codons 12, 13 or 61 are the most commonly found mutations in lung cancer (34). They are also commonly found in colorectal cancers where they have shown to predict negative benefit from anti-EGFR therapies (35.), including cetuximab (ERBITUX®, made by ImClone LLC, a wholly-owned subsidiary of Eli Lilly and Co).

BRAF—

SNPs in the codon 600 are reported in ˜50% of melanoma cases, making these the most common mutations in this type of cancer (36), (37). The FDA has approved use of the drug vemurafenib for melanoma patients with V600E mutations and there are additional BRAF linked therapies on the way (38.).

EGFR—

SNPs within EGFR have been shown to be an important for making therapeutic decisions in lung cancer. The presence of some SNPs (G719*, L585R and L861Q) have shown correlation with increased sensitivity to the EGFR targeted kinase inhibitors such as erlotinib (Tarceva) and gefitinib (Iressa) (39), (40). Other EGFR SNPs (T790M) can infer an acquired resistance to these targeted inhibitors (41), (42).

KIT—

SNPs in KIT are often found in melanoma but have also been report in lung cancer. Like EGFR, some KIT SNPs can signal sensitivity to targeted therapy while others infer a resistance to the drug. Melanoma patients with the SNPs V559A or V559D have been shown to respond to imatinib (43), (44), (45). Patients with the SNP D816H are not sensitive to imatinib or a similar kinase inhibitor sunitinib (46).

Medium Mutations (about 3 to about 300 base pairs in size)

Typical algorithms used to analyze Next-Generation Sequencing (NGS) data tend to struggle at detecting these mutations when they are at low-levels within the sample, as is the case with somatic mutations.

EGFR Deletions and Insertions—

In-frame deletion in Exon 19 of EGFR are one of the most commonly found types of mutation in lung cancer but insertions in exon 19 and exon 20 are also reported (47), (48). Insertions and deletions in exon 19 are correlated with sensitivity to the EGFR inhibitors erlotinib and gefitinib (49), (50) while insertions in exon 20 are correlated with a lack of sensitivity to these drugs (51).

ERBB2 Insertions—

Insertions in exon 20 of ERBB2 (or HER2) have been reported in 2-4% of Non-Small Cell Lung Cancer (NSCLC) (52), (53) cases and in up to 6% of NSCLC patients that are negative for KRAS, EGFR and ALK mutations (54). Pre-clinical studies have suggested that ERBB2 Insertions may be a correlated with resistance to the EGFR tyrosine kinase inhibitors erlotinib and gefitinib (55). More recent studies have shown ERBB2 positive patients responding positively to the anti-HER2 antibody trastuzumab (56), a humanized monoclonal antibody that had previously proven ineffective in an un-selected population (57), (58).

FLT3 Internal Tandem Duplications (ITDs)—

FLT3 ITDs are one of the most common type of mutation that is found in Acute Myeloid Leukemia (AML) (59) and are generally correlated with poor prognosis for the patient (60), (61). The mutations are almost always repetitions of FLT3 coding sequence inserted into either exon 14 or 15; they can range in size from about 3 base-pairs (bp) to about 300 bp. This variation in size can make it difficult for a single test or technology to detect the full spectrum of ITDs. Recent studies suggest that FLT3 positive patients make be sensitive to treatment with the TKI's sorafenib (62) and quizartinib (63.).

Large Mutations (about 300-about 300,000,000+ base pairs in size)

Large rearrangements of chromosomes are currently impossible to test using NGS and tradition PCR-based enrichment techniques. It is possible to detect these types of mutations using hybridization based pull-down techniques (64), these methods are expensive, require a large amount of DNA and can be insensitive.

EML4-ALK Fusions—

EML4-ALK fused proteins are a common biomarker found in NSCLC; they are generated by a about 12,000,000 bp sized inversion mutation on chromosome 2 where a chunk of the chromosome has flipped around connecting the EML4 gene to the ALK gene. Cancers driven by ALK fusion are sensitive to ALK targeted TKIs such as crizotinib (64) as well as 2^{nd} generation ALK inhibitor ceritinib (66).

The method employed included two PCR reactions followed by sequencing-by-synthesis (SBS) on an NGS instrument. The raw DNA sequence reads are then analyzed to find low level mutations and determine if they are present at a level that is above the background level of sequence errors produced during PCR or SBS. The mutation detection process/software detects each of the three mutation types described above (small, medium and large) using a different mechanism, each of which is described herein.

The first PCR reaction is target-specific and is performed on genomic DNA extracted from human tissue. For this cancer test, there are two separate target-specific PCR reactions, each with each with a unique set PCR primers, or Probe Set. A portion of the primers in each Probe Set are intended to detect the small and medium sized mutations. These primers are designed to flank regions in the sample's genomic DNA that contain the mutations described in FIG. 13 (except for EML4-ALK). Special care is taken to minimize the amount of overlap in the size of amplicon each primer pair is expected to produce in a canonical sample. Thus it is intended that each primer pair in a reaction produces a product that is at least 2 bp different in size from every other amplicon produced by the other primer pairs in the reaction. The 16 targets in Probe Set A and 14 targets in Probe Set B and their respective amplicon sizes are shown in Tables 7a and 7b.

TABLE 7a
Sixteen targets of Probe Set A and their respective amplicon sizes

| Name | Size | Probe Set |
|---|---|---|
| EGFR Indel Target Region 1 Exon 19 | 171 | A |
| EGFR SNPs G719* | 181 | A |
| KRAS SNPs G12* and G13* | 172 | A |
| HER2 insertions | 174 | A |
| PTEN indels | 178 | A |
| PTEN R173 SNPs | 148 | A |
| TP53 Region2 SNPs 488-536 | 175 | A |
| TP53 Region4 SNPs 701-747 | 200 | A |
| TP53 Region5 SNPs 814-853 | 151 | A |
| PIK3CA Regionl SNPs 1616-1659 | 140 | A |
| PIK3CA Region2 SNPs 3062-3145 | 190 | A |
| KIT Indel Region2 V559del | 162 | A |
| KIT SNP Region D816V | 160 | A |
| NPM1 Indels | 210 | A |
| FLT3 ITDs Exon 14 | 207 | A |
| EGFR SNPs T790M | 175 | A |

TABLE 7b
Fourteen targets of Probe Set B and their respective amplicon sizes

| Name | Size | Probe Set |
|---|---|---|
| EGFR Indel Target Region 2 Exon 19 | 180 | B |
| EGFR Indel Target Region 3 Exon 20 | 177 | B |
| EGFR SNPs T790M | 175 | B |
| EGFR SNPs L858R and L861Q | 168 | B |
| KRAS SNPs Q61* | 151 | B |
| PTEN R130 SNPs | 179 | B |
| PTEN R233 SNPs | 173 | B |
| BRAF SNPs around V600 | 152 | B |
| TP53 Regionl SNPs 422-488 | 161 | B |
| TP53 Region3 SNPs 586-659 | 157 | B |
| PDGFRA deletion | 160 | B |
| KIT Indel Region1 S503ins | 146 | B |
| FLT3 ITDs Exon 15 | 170 | B |
| FLT3 SNPs Exon 20 | 190 | B |

Each Probe Set also contains 78 Dummy primers that are used to detect the presence of inversions in chromosome 2 that cause EML4-ALK fusions. One reaction contains the positive strand primers of primer pairs falling in across ALK intron 19 and the negative strand primers of primer pairs falling across EML4 introns 6, 12 and 18. The other reaction contains the opposite, the negative strand primers of primer pairs falling in across ALK intron 19 and the positive strand primers of primer pairs falling across EML4 introns 13, 6 and 18. In canonical samples that do not contain the chromosome 2 inversion, the dummy primers in each reaction do not result in PCR amplicons. In samples containing the chromosomal inversions that connect ALK intron 19 to EML4 introns 13, 6 or 18 (EML4-ALK variants 1, 3a/b and 5 respectively) the dummy primers on ALK and EML4 are in the right orientation to produce PCR amplicons which are detected and identified by the sequence analysis process described herein. A summary of the Dummy primers in each Probe Set is included in Table 8.

TABLE 8
EML4-ALK Dummy Primers in Each Probe Set

| Probe Set A | Probe Set B |
|---|---|
| ALK Intron19 pos 23-270 Positive Strand | ALK Intron19 pos 23-270 Negative Strand |
| ALK Intron19 pos 265-534 Positive Strand | ALK Intron19 pos 265-534 Negative Strand |
| ALK Intron19 pos 513-763 Positive Strand | ALK Intron19 pos 513-763 Negative Strand |
| ALK Intron19 pos 737-1002 Positive Strand | ALK Intron19 pos 737-1002 Negative Strand |
| ALK Intron19 pos 983-1254 Positive Strand | ALK Intron19 pos 983-1254 Negative Strand |
| ALK Intron19 pos 1227-1515 Positive Strand | ALK Intron19 pos 1227-1515 Negative Strand |
| ALK Intron19 pos 1457-1730 Positive Strand | ALK Intron19 pos 1457-1730 Negative Strand |
| ALK Intron19 pos 1709-1966 Positive Strand | ALK Intron19 pos 1709-1966 Negative Strand |
| EML4 Intron 13 pos 97-361 Negative Strand | EML4 Intron 13 pos 97-361 Positive Strand |
| EML4 Intron 13 pos 341-616 Negative Strand | EML4 Intron 13 pos 341-616 Positive Strand |
| EML4 Intron 13 pos 440-699 Negative Strand | EML4 Intron 13 pos 440-699 Positive Strand |
| EML4 Intron 13 pos 678-959 Negative Strand | EML4 Intron 13 pos 678-959 Positive Strand |
| EML4 Intron 13 pos 936-1194 Negative Strand | EML4 Intron 13 pos 936-1194 Positive Strand |
| EML4 Intron 13 pos 1181-1474 Negative Strand | EML4 Intron 13 pos 1181-1474 Positive Strand |
| EML4 Intron 13 pos 1463-1762 Negative Strand | EML4 Intron 13 pos 1463-1762 Positive Strand |
| EML4 Intron 13 pos 1754-2020 Negative Strand | EML4 Intron 13 pos 1754-2020 Positive Strand |
| EML4 Intron 13 pos 1985-2273 Negative Strand | EML4 Intron 13 pos 1985-2273 Positive Strand |
| EML4 Intron 13 pos 2137-2390 Negative Strand | EML4 Intron 13 pos 2137-2390 Positive Strand |
| EML4 Intron 13 pos 2354-2616 Negative Strand | EML4 Intron 13 pos 2354-2616 Positive Strand |
| EML4 Intron 13 pos 2526-2755 Negative Strand | EML4 Intron 13 pos 2526-2755 Positive Strand |
| EML4 Intron 13 pos 2686-2980 Negative Strand | EML4 Intron 13 pos 2686-2980 Positive Strand |
| EML4 Intron 13 pos 2890-3113 Negative Strand | EML4 Intron 13 pos 2890-3113 Positive Strand |
| EML4 Intron 13 pos 3080-3361 Negative Strand | EML4 Intron 13 pos 3080-3361 Positive Strand |
| EML4 Intron 13 pos 3335-3594 Negative Strand | EML4 Intron 13 pos 3335-3594 Positive Strand |
| EML4 Intron 13 pos 3522-3821 Negative Strand | EML4 Intron 13 pos 3522-3821 Positive Strand |
| EML4 Intron 13 pos 3793-4111 Negative Strand | EML4 Intron 13 pos 3793-4111 Positive Strand |
| EML4 Intron 13 pos 4246-4537 Negative Strand | EML4 Intron 13 pos 4246-4537 Positive Strand |
| EML4 Intron 13 pos 4590-4859 Negative Strand | EML4 Intron 13 pos 4590-4859 Positive Strand |
| EML4 Intron 13 pos 4835-5123 Negative Strand | EML4 Intron 13 pos 4835-5123 Positive Strand |
| EML4 Intron 13 pos 5179-5429 Negative Strand | EML4 Intron 13 pos 5179-5429 Positive Strand |
| EML4 Intron 13 pos 5435-5711 Negative Strand | EML4 Intron 13 pos 5435-5711 Positive Strand |
| EML4 Intron6 pos 94-355 Negative Strand | EML4 Intron6 pos 94-355 Positive Strand |
| EML4 Intron6 pos 7240-7506 Negative Strand | EML4 Intron6 pos 7240-7506 Positive Strand |
| EML4 Intron6 pos 11444-11648 Negative Strand | EML4 Intron6 pos 11444-11648 Positive Strand |
| EML4 Intron6 pos 5465-5775 Negative Strand | EML4 Intron6 pos 5465-5775 Positive Strand |
| EML4 Intron6 pos 12004-12307 Negative Strand | EML4 Intron6 pos 12004-12307 Positive Strand |
| EML4 Intron6 pos 9806-10104 Negative Strand | EML4 Intron6 pos 9806-10104 Positive Strand |
| EML4 Intron6 pos 2960-3110 Negative Strand | EML4 Intron6 pos 2960-3110 Positive Strand |
| EML4 Intron 18 pos 402-701 Negative Strand | EML4 Intron 18 pos 402-701 Positive Strand |

The primers used in this first PCR step contain a target specific region that is complementary to the DNA flanking the genomic regions it is intended to amplify as well a 33 bp adapter sequence that is appended at the 5′ end of the target specific region. After the first round of target-specific PCR, the samples are purified before undergoing a second amplification using sequencer specific primers that hybridized to the sequencer adapter region of the original PCR primers that have now been incorporated into the amplicons produced by the first PCR reaction. Each sequencer specific pair contains sequence required for hybridizing to the SBS instrument's flowcell for sequence analysis as well as index sequences that allow multiple samples to be pooled together for a run and then de-multiplexed in the analysis. After the Index PCR each sample is quantified separately and then they are pooled together in an equimolar fashion and loaded onto the instrument. Analysis of the FASTQ data files that are output by the sequencer is performed by the sequence analysis methods described herein.

Materials and Methods

Reagents:

Sequences of all adapters and primers used in the test are provided in FIG. 14 and Tables 9 and 10.

TABLE 9
Full Sequences of Small and Medium Mutation Primers with Sequencer Adapters

| Pos | Name | Sequence |
|---|---|---|
| A01 | EGFR Indel Target Region | TCGTCGGCAGCGTCAGATGTGTATAAGAGACAGGCA |
| | 1 Exon 19 LEFT | CCATCTCACAATTGCCAGTTAAcgt (SEQ ID NO: 67) |
| B01 | EGFR Indel Target Region | GTCTCGTGGGCTCGGAGATGTGTATAAGAGACAGacac |
| | 1 Exon 19 RIGHT | agcaaagcagaaactcacATCG (SEQ ID NO: 68) |
| C01 | EGFR Indel Target Region | TCGTCGGCAGCGTCAGATGTGTATAAGAGACAGTGC |
| | 2 Exon 19 LEFT | CAGTTAAcgtcttccttctctct (SEQ ID NO: 69) |
| D01 | EGFR Indel Target Region | GTCTCGTGGGCTCGGAGATGTGTATAAGAGACAGTG |
| | 2 Exon 19 RIGHT | AGGTTCAGAGCCATGGACCc (SEQ ID NO: 70) |
| E01 | EGFR Indel Target Region | TCGTCGGCAGCGTCAGATGTGTATAAGAGACAGCAT |
| | 3 Exon 20 LEFT | TCATGCGTCTTCACCTGGAA (SEQ ID NO: 71) |
| F01 | EGFR Indel Target Region | GTCTCGTGGGCTCGGAGATGTGTATAAGAGACAGGT |
| | 3 Exon 20 RIGHT | GATGAGCTGCACGGTGGA (SEQ ID NO: 72) |
| G01 | EGFR SNP T790M LEFT | TCGTCGGCAGCGTCAGATGTGTATAAGAGACAGCAT |
| | | TCATGCGTCTTCACCTGGAA (SEQ ID NO: 73) |
| H01 | EGFR SNP T790M RIGHT | GTCTCGTGGGCTCGGAGATGTGTATAAGAGACAGGT |
| | | GATGAGCTGCACGGTGGA (SEQ ID NO: 74) |
| A02 | EGFR SNPs G719* LEFT | TCGTCGGCAGCGTCAGATGTGTATAAGAGACAGAGC |
| | | ATGGTGAGGGCTGAGGTGA (SEQ ID NO: 75) |
| B02 | EGFR SNPs G719* | GTCTCGTGGGCTCGGAGATGTGTATAAGAGACAGcctta |
| | RIGHT | cCTTATACACCGTGCCGAAC (SEQ ID NO: 76) |
| C02 | EGFR SNPs L858R and | TCGTCGGCAGCGTCAGATGTGTATAAGAGACAGTGA |
| | L861Q LEFT | AAACACCGCAGCATGTCAAGAT (SEQ ID NO: 77) |
| D02 | EGFR SNPs L858R and | GTCTCGTGGGCTCGGAGATGTGTATAAGAGACAGAC |
| | L861Q RIGHT | AATACAGCTAGTGGGAAGGCAGCC (SEQ ID NO: 78) |
| E02 | KRAS SNPs G12* and | TCGTCGGCAGCGTCAGATGTGTATAAGAGACAGgGCC |
| | G13* LEFT | TGCTGAAAATGACTGAA (SEQ ID NO: 79) |
| F02 | KRAS SNPs G12* and | GTCTCGTGGGCTCGGAGATGTGTATAAGAGACAGTC |
| | G13* RIGHT | AAAGAATGGTCCTGCACCAGTAa (SEQ ID NO: 80) |
| G02 | KRAS SNPs Q61* LEFT | TCGTCGGCAGCGTCAGATGTGTATAAGAGACAGTccag |
| | | actgtgtttctcccttc (SEQ ID NO: 81) |
| H02 | KRAS SNPs Q61* RIGHT | GTCTCGTGGGCTCGGAGATGTGTATAAGAGACAGAG |
| | | AAAGCCCTCCCCAGTCCTCA (SEQ ID NO: 82) |
| A03 | HER2 insertions LEFT | TCGTCGGCAGCGTCAGATGTGTATAAGAGACAGGGG |
| | | CCATGGCTGTGGTTTGT (SEQ ID NO: 83) |
| B03 | HER2 insertions RIGHT | GTCTCGTGGGCTCGGAGATGTGTATAAGAGACAGCA |
| | | GCTGCACCGTGGATGTCA (SEQ ID NO: 84) |
| C03 | PTEN indels LEFT | TCGTCGGCAGCGTCAGATGTGTATAAGAGACAGACC |
| | | AGGACCAGAGGAAACCTCA (SEQ ID NO: 85) |
| D03 | PTEN indels RIGHT | GTCTCGTGGGCTCGGAGATGTGTATAAGAGACAGTG |
| | | GAGAAAAGTATCGGTTGGCTTTG (SEQ ID NO: 86) |
| E03 | PTEN R130 SNPs LEFT | TCGTCGGCAGCGTCAGATGTGTATAAGAGACAGCCTT |
| | | TTGAAGACCATAACCCACCAC (SEQ ID NO: 87) |
| F03 | PTEN R130 SNPs RIGHT | GTCTCGTGGGCTCGGAGATGTGTATAAGAGACAGGC |
| | | CTTTAAAAATTTGCCCCGATGT (SEQ ID NO: 88) |
| G03 | PTEN R173 SNPs LEFT | TCGTCGGCAGCGTCAGATGTGTATAAGAGACAGcacca |
| | | gGGAGTAACTATTCCCAGTCA (SEQ ID NO: 89) |
| H03 | PTEN R173 SNPs RIGHT | GTCTCGTGGGCTCGGAGATGTGTATAAGAGACAGTG |
| | | CAAGTTCCGCCACTGAACA (SEQ ID NO: 90) |
| A04 | PTEN R233 SNPs LEFT | TCGTCGGCAGCGTCAGATGTGTATAAGAGACAGCAG |
| | | TTTGACAGTTAAAGGCATTTCC (SEQ ID NO: 91) |
| B04 | PTEN R233 SNPs RIGHT | GTCTCGTGGGCTCGGAGATGTGTATAAGAGACAGCA |
| | | CAGGTAACGGCTGAGGGAAC (SEQ ID NO: 92) |
| C04 | BRAF SNPs around V600 | TCGTCGGCAGCGTCAGATGTGTATAAGAGACAGCTCT |
| | LEFT | TCATAATGCTTGCTCTGATAGGA (SEQ ID NO: 93) |
| D04 | BRAF SNPs around V600 | GTCTCGTGGGCTCGGAGATGTGTATAAGAGACAGTG |
| | RIGHT | ATGGGACCCACTCCATCG (SEQ ID NO: 94) |
| E04 | TP53 Regionl SNPs 422- | TCGTCGGCAGCGTCAGATGTGTATAAGAGACAGccttcc |
| | 488 LEFT | tcttcctacagTACTCCCCT (SEQ ID NO: 95) |
| F04 | TP53 Regionl SNPs 422- | GTCTCGTGGGCTCGGAGATGTGTATAAGAGACAGCA |
| | 488 RIGHT | CAACCTCCGTCATGTGCTG (SEQ ID NO: 96) |
| G04 | TP53 Region2 SNPs 488- | TCGTCGGCAGCGTCAGATGTGTATAAGAGACAGCCC |
| | 536 LEFT | TGTGCAGCTGTGGGTTGATT (SEQ ID NO: 97) |
| H04 | TP53 Region2 SNPs 488- | GTCTCGTGGGCTCGGAGATGTGTATAAGAGACAGCA |
| | 536 RIGHT | ACCAGCCCTGTcgtctct (SEQ ID NO: 98) |
| A05 | TP53 Region3 SNPs 586- | TCGTCGGCAGCGTCAGATGTGTATAAGAGACAGcctcac |
| | 659 LEFT | tgattgctcttagGTCTGGC (SEQ ID NO: 99) |
| B05 | TP53 Region3 SNPs 586- | GTCTCGTGGGCTCGGAGATGTGTATAAGAGACAGcaga |
| | 659 RIGHT | gaccccagttgcaaaccagac (SEQ ID NO: 100) |
| C05 | TP53 Region4 SNPs 701- | TCGTCGGCAGCGTCAGATGTGTATAAGAGACAGGGC |
| | 747 LEFT | TCTGACTGTACCACCATCCAC (SEQ ID NO: 101) |
| D05 | TP53 Region4 SNPs 701- | GTCTCGTGGGCTCGGAGATGTGTATAAGAGACAGGA |
| | 747 RIGHT | AGAAATCGGTAAGAGGTGGGCC (SEQ ID NO: 102) |
| E05 | TP53 Region5 SNPs 814- | TCGTCGGCAGCGTCAGATGTGTATAAGAGACAGTGG |
| | 853 LEFT | GACGGAACAGCTTTGAG (SEQ ID NO: 103) |
| F05 | TP53 Region5 SNPs 814- | GTCTCGTGGGCTCGGAGATGTGTATAAGAGACAGaccg |
| | 853 RIGHT | cttcttgtcctgcttgctta (SEQ ID NO: 104) |
| G05 | PDGFRA deletion LEFT | TCGTCGGCAGCGTCAGATGTGTATAAGAGACAGATT |
| | | GTGAAGATCTGTGACTTTGGCC (SEQ ID NO: 105) |
| H05 | PDGFRA deletion RIGHT | GTCTCGTGGGCTCGGAGATGTGTATAAGAGACAGAG |
| | | GCACCGAATCTCTAGAAGCAACA (SEQ ID NO: 106) |
| A06 | PIK3CA Regionl SNPs | TCGTCGGCAGCGTCAGATGTGTATAAGAGACAGACT |
| | 1616-1659 LEFT | AGCTAGAGACAATGAATTAAGGGA |
| | | (SEQ ID NO: 107) |
| B06 | PIK3CA Regionl SNPs | GTCTCGTGGGCTCGGAGATGTGTATAAGAGACAGagaa |
| | 1616-1659 RIGHT | tctccattttagcacttacCT (SEQ ID NO: 108) |
| C06 | PIK3CA Region2 SNPs | TCGTCGGCAGCGTCAGATGTGTATAAGAGACAGAAT |
| | 3062-3145 LEFT | GATGCTTGGCTCTGGAATGCC (SEQ ID NO: 109) |
| D06 | PIK3CA Region2 SNPs | GTCTCGTGGGCTCGGAGATGTGTATAAGAGACAGTG |
| | 3062-3145 RIGHT | CATGCTGTTTAATTGTGTGGAAG (SEQ ID NO: 110) |
| E06 | KIT Indel Regionl S503 ins | TCGTCGGCAGCGTCAGATGTGTATAAGAGACAGCAA |
| | LEFT | TGGCACGGTTGAATGTAAGGCTT (SEQ ID NO: 111) |
| F06 | KIT Indel Regionl S503 ins | GTCTCGTGGGCTCGGAGATGTGTATAAGAGACAGTG |
| | RIGHT | ACTGATATGGTAGACAGAGCCTAAA |
| | | (SEQ ID NO: 112) |
| G06 | KIT Indel Region2 | TCGTCGGCAGCGTCAGATGTGTATAAGAGACAGtctccc |
| | V559del LEFT | cacagAAACCCATGTATGA (SEQ ID NO: 113) |
| H06 | KIT Indel Region2 | GTCTCGTGGGCTCGGAGATGTGTATAAGAGACAGggaa |
| | V559del RIGHT | agcccctgtttcatactgacC (SEQ ID NO: 114) |
| A07 | KIT SNP Region D816V | TCGTCGGCAGCGTCAGATGTGTATAAGAGACAGtttctttt |
| | LEFT | ctcctccaacctaatagTGT (SEQ ID NO: 115) |
| B07 | KIT SNP Region D816V | GTCTCGTGGGCTCGGAGATGTGTATAAGAGACAGatgg |
| | RIGHT | gtactcacGTTTCCTTTAACC (SEQ ID NO: 116) |
| C07 | NPM1 Indels LEFT | TCGTCGGCAGCGTCAGATGTGTATAAGAGACAGTGA |
| | | TGTCTATGAAGTGTTGTGGTTCCT (SEQ ID NO: 117) |
| D07 | NPM1 Indels RIGHT | GTCTCGTGGGCTCGGAGATGTGTATAAGAGACAGCA |
| | | AACACGGTAGGGAAAGTTCTCAC (SEQ ID NO: 118) |
| E07 | FLT3 ITDs Exon14 LEFT | TCGTCGGCAGCGTCAGATGTGTATAAGAGACAGAAC |
| | | TGCCTATTCCTAActgactcatc (SEQ ID NO: 119) |
| F07 | FLT3 ITDs Exon14 | GTCTCGTGGGCTCGGAGATGTGTATAAGAGACAGGC |
| | RIGHT | TGCAGAaacatttggcaca (SEQ ID NO: 120) |
| G07 | FLT3 ITDs Exon 15 LEFT | TCGTCGGCAGCGTCAGATGTGTATAAGAGACAGTGCa |
| | | cgtactcaccatttgtctttgca (SEQ ID NO: 121) |
| H07 | FLT3 ITDs Exon 15 | GTCTCGTGGGCTCGGAGATGTGTATAAGAGACAGTG |
| | RIGHT | TGCATCTTTGttgctgtcctt (SEQ ID NO: 122) |
| A08 | FLT3 SNPs Exon 20 LEFT | TCGTCGGCAGCGTCAGATGTGTATAAGAGACAGTTG |
| | | CACTCCAGGATAATACACATCACA |
| | | (SEQ ID NO: 123) |
| B08 | FLT3 SNPs Exon 20 | GTCTCGTGGGCTCGGAGATGTGTATAAGAGACAGcagc |
| | RIGHT | ctcacATTGCCCCTGACA (SEQ ID NO: 124) |

*indicates that there are multiple possible mutations at a particular codon. For example, the codon for G719* can contain numerous mutations that result in different amino acid changes, for example, G7195, G719C, etc.

TABLE 10
Full Sequences of EML4-ALK Dummy Primers with Sequencer Adapters

| Pos | Name | Sequence |
|---|---|---|
| A01 | ALK Intron19 pos 23- | TCGTCGGCAGCGTCAGATGTGTATAAGAGACAGGGCTTT |
| | 270 LEFT | CTCCGGCATCATGAtt (SEQ ID NO: 125) |
| B01 | ALK Intron19 pos 23- | GTCTCGTGGGCTCGGAGATGTGTATAAGAGACAGTGAG |
| | 270 RIGHT | GTGCAGAATCAGGGGCTC (SEQ ID NO: 126) |
| C01 | ALK Intron19 pos 265- | TCGTCGGCAGCGTCAGATGTGTATAAGAGACAGACCTCA |
| | 534 LEFT | GCCCCGTGTGTATCCT (SEQ ID NO: 127) |
| D01 | ALK Intron19 pos 265- | GTCTCGTGGGCTCGGAGATGTGTATAAGAGACAGTCAGC |
| | 534 RIGHT | TCACCTTGGCTCACAGG (SEQ ID NO: 128) |
| E01 | ALK Intron19 pos 513- | TCGTCGGCAGCGTCAGATGTGTATAAGAGACAGCCTGTG |
| | 763 LEFT | AGCCAAGGTGAGCTGA (SEQ ID NO: 129) |
| F01 | ALK Intron19 pos 513- | GTCTCGTGGGCTCGGAGATGTGTATAAGAGACAGAGCTC |
| | 763 RIGHT | CTATTATCCTGTCCCTTTGA (SEQ ID NO: 130) |
| G01 | ALK Intron19 pos 737- | TCGTCGGCAGCGTCAGATGTGTATAAGAGACAGCCTCAA |
| | 1002 LEFT | AGGGACAGGATAATAGGAGCT (SEQ ID NO: 131) |
| H01 | ALK Intron19 pos 737- | GTCTCGTGGGCTCGGAGATGTGTATAAGAGACAGAGGA |
| | 1002 RIGHT | TGTTCTGGAAGGCAAACTCCA (SEQ ID NO: 132) |
| A02 | ALK Intron19 pos 983- | TCGTCGGCAGCGTCAGATGTGTATAAGAGACAGTTTGCC |
| | 1254 LEFT | TTCCAGAACATCCTCACAT (SEQ ID NO: 133) |
| B02 | ALK Intron19 pos 983- | GTCTCGTGGGCTCGGAGATGTGTATAAGAGACAGCTGG |
| | 1254 RIGHT | GGATCTGTGCTCTAATTCCGC (SEQ ID NO: 134) |
| C02 | ALK Intron19 pos | TCGTCGGCAGCGTCAGATGTGTATAAGAGACAGGAGGC |
| | 1227-1515 LEFT | GGAATTAGAGCACAGATCCC (SEQ ID NO: 135) |
| D02 | ALK Intron19 pos | GTCTCGTGGGCTCGGAGATGTGTATAAGAGACAGCCTAA |
| | 1227-1515 RIGHT | GGAAGTTTCAGCAAGGCCCT (SEQ ID NO: 136) |
| E02 | ALK Intron19 pos | TCGTCGGCAGCGTCAGATGTGTATAAGAGACAGCCTCTG |
| | 1457-1730 LEFT | ATGACTGACTTTGGCTCCA (SEQ ID NO: 137) |
| F02 | ALK Intron19 pos | GTCTCGTGGGCTCGGAGATGTGTATAAGAGACAGAGAG |
| | 1457-1730 RIGHT | TCATGTTAGTCTGGTTCCTCC (SEQ ID NO: 138) |
| G02 | ALK Intron19 pos | TCGTCGGCAGCGTCAGATGTGTATAAGAGACAGGGAAC |
| | 1709-1966 LEFT | CAGACTAACATGACTCTGCCC (SEQ ID NO: 139) |
| H02 | ALK Intron19 pos | GTCTCGTGGGCTCGGAGATGTGTATAAGAGACAGTGGTC |
| | 1709-1966 RIGHT | AGCTGCAACATGGCCTG (SEQ ID NO: 140) |
| A03 | EML4 Intron 13 pos | TCGTCGGCAGCGTCAGATGTGTATAAGAGACAGAAAAC |
| | 97-361 LEFT | TACTGTAGAGCCCACACCTG (SEQ ID NO: 141) |
| B03 | EML4 Intron 13 pos | GTCTCGTGGGCTCGGAGATGTGTATAAGAGACAGGGGA |
| | 97-361 RIGHT | ATGGTTCAGTATAGTCAAATGTGGGT (SEQ ID NO: 142) |
| C03 | EML4 Intron 13 pos | TCGTCGGCAGCGTCAGATGTGTATAAGAGACAGTGACTA |
| | 341-616 LEFT | TACTGAACCATTCCCTTTAGG (SEQ ID NO: 143) |
| D03 | EML4 Intron 13 pos | GTCTCGTGGGCTCGGAGATGTGTATAAGAGACAGTTGAG |
| | 341-616 RIGHT | GTAAAGCTGAATGGATGCC (SEQ ID NO: 144) |
| E03 | EML4 Intron 13 pos | TCGTCGGCAGCGTCAGATGTGTATAAGAGACAGGCTGGT |
| | 440-699 LEFT | TCTGGGATATCTGTTAGAGCA (SEQ ID NO: 145) |
| F03 | EML4 Intron 13 pos | GTCTCGTGGGCTCGGAGATGTGTATAAGAGACAGCCGTA |
| | 440-699 RIGHT | GAAAAGGGCAAAGAGGA (SEQ ID NO: 146) |
| G03 | EML4 Intron 13 pos | TCGTCGGCAGCGTCAGATGTGTATAAGAGACAGTCCTCT |
| | 678-959 LEFT | TTGCCCTTTTCTACGGTAAA (SEQ ID NO: 147) |
| H03 | EML4 Intron 13 pos | GTCTCGTGGGCTCGGAGATGTGTATAAGAGACAGTGGTG |
| | 678-959 RIGHT | AATTATTGAGGACTGGCTGACC (SEQ ID NO: 148) |
| A04 | EML4 Intron 13 pos | TCGTCGGCAGCGTCAGATGTGTATAAGAGACAGCAGCC |
| | 936-1194 LEFT | AGTCCTCAATAATTCACCAA (SEQ ID NO: 149) |
| B04 | EML4 Intron 13 pos | GTCTCGTGGGCTCGGAGATGTGTATAAGAGACAGTTCTG |
| | 936-1194 RIGHT | AGCACACGAACAGGGATTCC (SEQ ID NO: 150) |
| C04 | EML4 Intron 13 pos | TCGTCGGCAGCGTCAGATGTGTATAAGAGACAGTCGTGT |
| | 1181-1474 LEFT | GCTCAGAAAGACCCGATTT (SEQ ID NO: 151) |
| D04 | EML4 Intron 13 pos | GTCTCGTGGGCTCGGAGATGTGTATAAGAGACAGAGTC |
| | 1181-1474 RIGHT | AAGGAGCATCTGAATATCTGTC (SEQ ID NO: 152) |
| E04 | EML4 Intron 13 pos | TCGTCGGCAGCGTCAGATGTGTATAAGAGACAGTGCTCC |
| | 1463-1762 LEFT | TTGACTTCTGGATGGCATT (SEQ ID NO: 153) |
| F04 | EML4 Intron 13 pos | GTCTCGTGGGCTCGGAGATGTGTATAAGAGACAGCTGCC |
| | 1463-1762 RIGHT | ATTCTTCTCGTGTGTAAATT (SEQ ID NO: 154) |
| G04 | EML4 Intron 13 pos | TCGTCGGCAGCGTCAGATGTGTATAAGAGACAGGAATG |
| | 1754-2020 LEFT | GCAGTGGGTTGAGGGTTCTT (SEQ ID NO: 155) |
| H04 | EML4 Intron 13 pos | GTCTCGTGGGCTCGGAGATGTGTATAAGAGACAGCTTCC |
| | 1754-2020 RIGHT | TGTCCCCAACCTGAACACAA (SEQ ID NO: 156) |
| A05 | EML4 Intron 13 pos | TCGTCGGCAGCGTCAGATGTGTATAAGAGACAGAGCCTT |
| | 1985-2273 LEFT | AATATTTGTGTTCAGGTTGGGG (SEQ ID NO: 157) |
| B05 | EML4 Intron 13 pos | GTCTCGTGGGCTCGGAGATGTGTATAAGAGACAGTGTGG |
| | 1985-2273 RIGHT | ACACTTACTCTGGTCTGGACT (SEQ ID NO: 158) |
| C05 | EML4 Intron 13 pos | TCGTCGGCAGCGTCAGATGTGTATAAGAGACAGGCCTGT |
| | 2137-2390 LEFT | GTTGTACTTTGCCACA (SEQ ID NO: 159) |
| D05 | EML4 Intron 13 pos | GTCTCGTGGGCTCGGAGATGTGTATAAGAGACAGGTCAC |
| | 2137-2390 RIGHT | AGTACACTCAAATTTTGGTTGG (SEQ ID NO: 160) |
| E05 | EML4 Intron 13 pos | TCGTCGGCAGCGTCAGATGTGTATAAGAGACAGGGCAG |
| | 2354-2616 LEFT | TCTTACCAACCAAAATTTGAGT (SEQ ID NO: 161) |
| F05 | EML4 Intron 13 pos | GTCTCGTGGGCTCGGAGATGTGTATAAGAGACAGACAA |
| | 2354-2616 RIGHT | ATACCTCATACCTACTTAAGAAACAGA |
| | | (SEQ ID NO: 162) |
| G05 | EML4 Intron 13 pos | TCGTCGGCAGCGTCAGATGTGTATAAGAGACAGTTCCAA |
| | 2526-2755 LEFT | ACCATTTCTTCCTTAAACATGA (SEQ ID NO: 163) |
| H05 | EML4 Intron 13 pos | GTCTCGTGGGCTCGGAGATGTGTATAAGAGACAGTCCTT |
| | 2526-2755 RIGHT | TGCAAGACTTAAGAATGGTGA (SEQ ID NO: 164) |
| A06 | EML4 Intron 13 pos | TCGTCGGCAGCGTCAGATGTGTATAAGAGACAGTGGGT |
| | 2686-2980 LEFT | AAGTGGAAGTTGAGAGTATCT (SEQ ID NO: 165) |
| B06 | EML4 Intron 13 pos | GTCTCGTGGGCTCGGAGATGTGTATAAGAGACAGTCCAA |
| | 2686-2980 RIGHT | ACTTAATCACAAACCTCACCCT (SEQ ID NO: 166) |
| C06 | EML4 Intron 13 pos | TCGTCGGCAGCGTCAGATGTGTATAAGAGACAGTATTTG |
| | 2890-3113 LEFT | GCAGGCAGTGTAAACTTGC (SEQ ID NO: 167) |
| D06 | EML4 Intron 13 pos | GTCTCGTGGGCTCGGAGATGTGTATAAGAGACAGGTCTT |
| | 2890-3113 RIGHT | TCTTATGGGCCTGTATTTCTG (SEQ ID NO: 168) |
| E06 | EML4 Intron 13 pos | TCGTCGGCAGCGTCAGATGTGTATAAGAGACAGTGAAA |
| | 3080-3361 LEFT | TATCAGAAATACAGGCCCAT (SEQ ID NO: 169) |
| F06 | EML4 Intron 13 pos | GTCTCGTGGGCTCGGAGATGTGTATAAGAGACAGAACA |
| | 3080-3361 RIGHT | CTTAAAATCCTCCCAGAATGA (SEQ ID NO: 170) |
| G06 | EML4 Intron 13 pos | TCGTCGGCAGCGTCAGATGTGTATAAGAGACAGCATCAT |
| | 3335-3594 LEFT | TCTGGGAGGATTTTAAGTGTTT (SEQ ID NO: 171) |
| H06 | EML4 Intron 13 pos | GTCTCGTGGGCTCGGAGATGTGTATAAGAGACAGTCTGA |
| | 3335-3594 RIGHT | GAGTACTACTGGCTTTATTTGGA (SEQ ID NO: 172) |
| A07 | EML4 Intron 13 pos | TCGTCGGCAGCGTCAGATGTGTATAAGAGACAGGCACA |
| | 3522-3821 LEFT | GGGAAATAAGCCTAGAATTTGCTTTT (SEQ ID NO: 173) |
| B07 | EML4 Intron 13 pos | GTCTCGTGGGCTCGGAGATGTGTATAAGAGACAGGGGC |
| | 3522-3821 RIGHT | TTGCTTGATTTGGAGGAGAAC (SEQ ID NO: 174) |
| C07 | EML4 Intron 13 pos | TCGTCGGCAGCGTCAGATGTGTATAAGAGACAGGCAAG |
| | 3793-4111 LEFT | TTCTCCTCCAAATCAAGCAA (SEQ ID NO: 175) |
| D07 | EML4 Intron 13 pos | GTCTCGTGGGCTCGGAGATGTGTATAAGAGACAGTGTCC |
| | 3793-4111 RIGHT | TTAGGTCAGATAGTGGT (SEQ ID NO: 176) |
| E07 | EML4 Intron 13 pos | TCGTCGGCAGCGTCAGATGTGTATAAGAGACAGTGGAG |
| | 4246-4537 LEFT | TCATACAATGTGTGGTC (SEQ ID NO: 177) |
| F07 | EML4 Intron 13 pos | GTCTCGTGGGCTCGGAGATGTGTATAAGAGACAGGGTC |
| | 4246-4537 RIGHT | AGATGTTTGAAACCAACC (SEQ ID NO: 178) |
| G07 | EML4 Intron 13 pos | TCGTCGGCAGCGTCAGATGTGTATAAGAGACAGGCCAC |
| | 4590-4859 LEFT | AGTGCCCAGCCTTCAC (SEQ ID NO: 179) |
| H07 | EML4 Intron 13 pos | GTCTCGTGGGCTCGGAGATGTGTATAAGAGACAGACCA |
| | 4590-4859 RIGHT | GGTTCAAAATGGGAAGGTAGA (SEQ ID NO: 180) |
| A08 | EML4 Intron 13 pos | TCGTCGGCAGCGTCAGATGTGTATAAGAGACAGTCTACC |
| | 4835-5123 LEFT | TTCCCATTTTGAACCTGGT (SEQ ID NO: 181) |
| B08 | EML4 Intron 13 pos | GTCTCGTGGGCTCGGAGATGTGTATAAGAGACAGAGCC |
| | 4835-5123 RIGHT | CAGTTTTCTTGTATACCCATAGCA (SEQ ID NO: 182) |
| C08 | EML4 Intron 13 pos | TCGTCGGCAGCGTCAGATGTGTATAAGAGACAGTTCTCT |
| | 5179-5429 LEFT | GCTAGTAGGTCAAAAGCCA (SEQ ID NO: 183) |
| D08 | EML4 Intron 13 p05 | GTCTCGTGGGCTCGGAGATGTGTATAAGAGACAGCAAA |
| | 5179-5429 RIGHT | GCTGTGACTAGGCTCAAGT (SEQ ID NO: 184) |
| E08 | EML4 Intron 13 pos | TCGTCGGCAGCGTCAGATGTGTATAAGAGACAGAGCTA |
| | 5435-5711 LEFT | ACTATGTGGTATCCCCTAAGCT (SEQ ID NO: 185) |
| F08 | EML4 Intron 13 pos | GTCTCGTGGGCTCGGAGATGTGTATAAGAGACAGGCAA |
| | 5435-5711 RIGHT | AGCAGGTAGTAAAGTTTAGGGT (SEQ ID NO: 186) |
| G08 | EML4 Intron6 pos 94- | TCGTCGGCAGCGTCAGATGTGTATAAGAGACAGAGTTCA |
| | 355 LEFT | TAGTAATCAAAGAAAAGTGCGTT (SEQ ID NO: 187) |
| H08 | EML4 Intron6 pos 94- | GTCTCGTGGGCTCGGAGATGTGTATAAGAGACAGTGGAT |
| | 355 RIGHT | TCATCTAGCTCAAATCACTGT (SEQ ID NO: 188) |
| A09 | EML4 Intron6 pos | TCGTCGGCAGCGTCAGATGTGTATAAGAGACAGACCTGT |
| | 7240-7506 LEFT | CTGTTGTCCCCACCTACTT (SEQ ID NO: 189) |
| B09 | EML4 Intron6 pos | GTCTCGTGGGCTCGGAGATGTGTATAAGAGACAGATGCC |
| | 7240-7506 RIGHT | ACTGATCAACCGCAACTCTT (SEQ ID NO: 190) |
| C09 | EML4 Intron6 pos | TCGTCGGCAGCGTCAGATGTGTATAAGAGACAGGTGTTG |
| | 11444-11648 LEFT | TGTGCAGGCCAAAGGTATG (SEQ ID NO: 191) |
| D09 | EML4 Intron6 pos | GTCTCGTGGGCTCGGAGATGTGTATAAGAGACAGAATA |
| | 11444-11648 RIGHT | CCCCATACCCCATCTCAGCGA (SEQ ID NO: 192) |
| E09 | EML4 Intron6 pos | TCGTCGGCAGCGTCAGATGTGTATAAGAGACAGCAGGA |
| | 5465-5775 LEFT | AAGGAAGGACAGTTGCCTCC (SEQ ID NO: 193) |
| F09 | EML4 Intron6 pos | GTCTCGTGGGCTCGGAGATGTGTATAAGAGACAGGCAA |
| | 5465-5775 RIGHT | GGCTCTGAACAAAAGCACCTG (SEQ ID NO: 194) |
| G09 | EML4 Intron6 pos | TCGTCGGCAGCGTCAGATGTGTATAAGAGACAGGCCAG |
| | 12004-12307 LEFT | TCTTGCAGTTAACAAAGCGT (SEQ ID NO: 195) |
| H09 | EML4 Intron6 pos | GTCTCGTGGGCTCGGAGATGTGTATAAGAGACAGGAAC |
| | 12004-12307 RIGHT | AGGCACAGCTCAGAACACCAT (SEQ ID NO: 196) |
| A10 | EML4 Intron6 pos | TCGTCGGCAGCGTCAGATGTGTATAAGAGACAGTCTCCC |
| | 9806-10104 LEFT | TTCTCCACTCTGCCTGAAT (SEQ ID NO: 197) |
| B10 | EML4 Intron6 pos | GTCTCGTGGGCTCGGAGATGTGTATAAGAGACAGATGCT |
| | 9806-10104 RIGHT | TGCCCTTCAGTTTCCTTGGG (SEQ ID NO: 198) |
| C10 | EML4 Intron6 pos | TCGTCGGCAGCGTCAGATGTGTATAAGAGACAGGATGT |
| | 2960-3110 LEFT | ACGCAGGGCAATCTCTGAGG (SEQ ID NO: 199) |
| D10 | EML4 Intron6 pos | GTCTCGTGGGCTCGGAGATGTGTATAAGAGACAGTGGA |
| | 2960-3110 RIGHT | GCACAACCCAGCAGAACTAG (SEQ ID NO: 200) |
| E10 | EML4 Intron 18 pos | TCGTCGGCAGCGTCAGATGTGTATAAGAGACAGAGGCC |
| | 402-701 LEFT | CTTCAAGTCCTTTAGAATCT (SEQ ID NO: 201) |
| F10 | EML4 Intron 18 pos | GTCTCGTGGGCTCGGAGATGTGTATAAGAGACAGAGATT |
| | 402-701 RIGHT | CTCTGGATCCTGTGCTAATG (SEQ ID NO: 202) |

Procedure:
1. Target Specific PCR on genomic DNA extracted from human tissue samples
   a. Reaction Components (25 μL total)
      10 ng of genomic DNA into each reaction of 2 reaction per sample
      5 μL of Probe Set (A or B)
      12.5 μL of 2× HotStarTaq Plus DNA Polymerase PCR master-mix
      N μL of Water to bring the total reaction volume to 25 μL
   b. PCR under the following conditions:
      95° C. for 5 minutes to activate the polymerase followed by
      25 cycles of:
         95° C. for 30 seconds
         60° C. for 90 seconds
         72° C. for 90 seconds
      Then 68° C. for 10 minutes for final extension
2. Purify the PCR reaction using AmpureXP magnetic beads by:
   a. Add 25 μL of well mixed, room temperature (rt) AmpureXP beads to each PCR reaction. Mix well and then incubate at rt for 2 minutes before placing on magnetic stand for 5 minutes.
   b. Once the solution has cleared, remove the supernatant and rinse with two subsequent 200 μL aliquots of 80% ethanol allowing 30 seconds for each wash.
   c. Remove as much of the last EtOH was with a 100 μL tip. Then switch to 10 μL tips and remove any remaining EtOH. Allow to air dry on the magnetic plate (which the samples are never removed from during the washing process) for 10 minutes.
   d. Remove from magnet and elute the beads in 30 μL of TE (10 mM Tris 1 mM EDTA pH 8.0) and mix thoroughly then incubate at rt for 5 minutes off the magnet before returning to the magnet to incubate at rt for 2 minutes.
   e. Once the solution has cleared, remove 25 μL of the supernatant and store in a fresh tube.
3. Index PCR on amplicons produced in step 1 and purified in step 2.
   a. Reaction Components (25 μL total)
      3.5 μL of water
      4 μL PCR produce from Step 2e
      2.5 μL of i5 primers (A5XX)
      2.5 μL of i7 primers (A7XX)
      12.5 μL of 2× KAPA HiFi HotStart DNA Polymerase PCR master-mix
   b. PCR under the following conditions:
      95° C. for 3 minutes to activate the polymerase followed by
      10 cycles of:
         95° C. for 30 seconds
         62° C. for 30 seconds
         72° C. for 60 seconds
      Then 72° C. for 5 minutes for final extension
4. Purify the indexed PCR reaction using AmpureXP magnetic beads by:
   a. Add 20 μL of well mixed, room temperature (rt) AmpureXP beads to each PCR reaction. Mix well and then incubate at rt for 2 minutes before placing on magnetic stand for 5 minutes.
   b. Once the solution has cleared, remove the supernatant and rinse with two subsequent 200 μL aliquots of 80% ethanol allowing 30 seconds for each wash.
   c. Remove as much of the last EtOH was with a 100 μL tip. Then switch to 10 μL tips and remove any remaining EtOH. Allow to air dry on the magnetic plate (which the samples are never removed from during the washing process) for 10 minutes.
   d. Remove from magnet and elute the beads in 30 μL of TE (10 mM Tris 1 mM EDTA pH 8.0) and mix thoroughly then incubate at rt for 5 minutes off the magnet before returning to the magnet to incubate at rt for 2 minutes.
   e. Once the solution has cleared, remove 25 μL of the supernatant and store in a fresh tube.
5. Quantify each sample and then pool together at 4 nM before loading on the Illumina sequencer.

Results

The Cancer Test was performed on genomic DNA derived from human cell lines; some cell lines are known to contain mutations that are test covers and other are known not to contain mutations that the test covers.

a) Small Mutations

The Cancer Test was used to analyze DNA samples known to contain 23 different SNPs in BRAF, EGFR, KRAS and KIT. FIG. 5 summarizes the small mutations that have been detected to date. Tables 11-15 show the ten most common reads found in 5 targeted regions, the total number of each unique read and its percentage of the whole. Mutations are detected by the presence of a significant number of reads above the statistically determined cutoff of random noise cause by errors during PCR and SMS. All the mutations below were detect at greater than 3 standard deviations above the statistical cutoff.

Tables 11a-11c: Detection of KRAS Codon 12 and 13 Mutations

| | | |
|---|---|---|
| Sequence Read by NGS Instrument | # of Reads | % of Total |

a. Canonical sample with no mutations; none were detected above the cutoff
Target Name:: KRAS SNPs G12* and G13*
Sample Name:: 12878 (KRAS canonical)

| | | |
|---|---|---|
| Perfect match with expected KRAS Canonical Sequence | 25413 | −85.65% |
| 1 bp difference from KRAS canonical; random error | 82 | −0.28% |
| 1 bp difference from KRAS canonical; random error | 62 | −0.21% |
| 1 bp difference from KRAS canonical; random error | 55 | −0.19% |
| 1 bp difference from KRAS canonical; random error | 53 | −0.18% |
| 1 bp difference from KRAS canonical; random error | 53 | −0.18% |
| 1 bp difference from KRAS canonical; random error | 48 | −0.16% |
| 1 bp difference from KRAS canonical; random error | 44 | −0.15% |
| 1 bp difference from KRAS canonical; random error | 44 | −0.15% |
| 1 bp difference from KRAS canonical; random error | 43 | −0.14% |

b. Sample contains two KRAS mutations; both detected at
greater than 3 standard deviations above cutoff
Target Name:: KRAS SNPs G12* and G13*
Sample Name:: HDx3 (KRAS G12S @ 5%; G13D @25%)

| | | |
|---|---|---|
| Perfect match with expected KRAS Canonical Sequence | 15229 | −59.20% |
| Perfect match with KRAS G13D sequence | 5335 | −20.74% |
| Perfect match with KRAS G12S sequence | 1193 | −4.64% |
| 1 bp difference from KRAS canonical; random error | 47 | −0.18% |
| 1 bp difference from KRAS canonical; random error | 43 | −0.17% |
| 1 bp difference from KRAS canonical; random error | 36 | −0.14% |
| 1 bp difference from KRAS canonical; random error | 29 | −0.11% |
| 1 bp difference from KRAS canonical; random error | 27 | −0.10% |
| 1 bp difference from KRAS canonical; random error | 27 | −0.10% |
| 1 bp difference from KRAS canonical; random error | 26 | −0.10% |

c. Sample contains 7 KRAS mutations; All were detected at
greater than 3 standard deviations above cutoff
Target Name:: KRAS SNPs G12* and G13*
Sample Name:: HDx7 (KRAS G12A, G12C, G12D, G12R, G12S and
G12V @ 1.3%; G13D @25%)

| | | |
|---|---|---|
| Perfect match with expected KRAS Canonical Sequence | 17151 | −57.79% |
| Perfect match with KRAS G13D sequence | 6339 | −21.36% |
| Perfect match with KRAS G12A sequence | 371 | −1.25% |
| Perfect match with KRAS G12C sequence | 369 | −1.24% |
| Perfect match with KRAS G12D sequence | 359 | −1.21% |
| Perfect match with KRAS G12V sequence | 324 | −1.09% |
| Perfect match with KRAS G12R sequence | 286 | −0.96% |
| Perfect match with KRAS G12S sequence | 242 | −0.82% |
| 1 bp difference from KRAS canonical; random error | 48 | −0.16% |
| 1 bp difference from KRAS canonical; random error | 41 | −0.14% |

Tables 12a-12c: Detection of KRAS Codon 61 Mutations

| | | |
|---|---|---|
| Sequence Read by NGS Instrument | # of Reads | % of Total |

12a. Wild-type sample with no mutations; none were detected above the cutoff
Target Name:: KRAS SNPs Q61*
Sample Name:: 12878 (KRAS canonical)

| | | |
|---|---|---|
| Perfect match with expected KRAS Canonical Sequence | 72774 | −89.37% |
| 1 bp difference from KRAS canonical; random error | 184 | −0.23% |
| 1 bp difference from KRAS canonical; random error | 166 | −0.20% |
| 1 bp difference from KRAS canonical; random error | 155 | −0.19% |
| 1 bp difference from KRAS canonical; random error | 139 | −0.17% |
| 1 bp difference from KRAS canonical; random error | 137 | −0.17% |
| 1 bp difference from KRAS canonical; random error | 114 | −0.14% |
| 1 bp difference from KRAS canonical; random error | 112 | −0.14% |
| 1 bp difference from KRAS canonical; random error | 102 | −0.13% |
| 1 bp difference from KRAS canonical; random error | 102 | −0.13% |

12b. Sample is canonical for KRAS Q61* mutations; none were detected above the cutoff
Target Name:: KRAS SNPs Q61*
Sample Name:: HDx3 (KRAS Q61* canonical)

| | | |
|---|---|---|
| Perfect match with expected KRAS Canonical Sequence | 37375 | −89.25% |
| 1 bp difference from KRAS canonical; random error | 101 | −0.24% |
| 1 bp difference from KRAS canonical; random error | 88 | −0.21% |
| 1 bp difference from KRAS canonical; random error | 77 | −0.18% |
| 1 bp difference from KRAS canonical; random error | 68 | −0.16% |
| 1 bp difference from KRAS canonical; random error | 64 | −0.15% |
| 1 bp difference from KRAS canonical; random error | 62 | −0.15% |
| 1 bp difference from KRAS canonical; random error | 61 | −0.15% |
| 1 bp difference from KRAS canonical; random error | 59 | −0.14% |
| 1 bp difference from KRAS canonical; random error | 59 | −0.14% |

12c. Sample contains 2 KRAS mutations; both were detected at
greater than 3 standard deviations above cutoff
Target Name:: KRAS SNPs Q61*
Sample Name:: HDx7 (KRAS Q61H and Q61L @ 1.3%)

| | | |
|---|---|---|
| Perfect match with expected KRAS Canonical Sequence | 55685 | −87.83% |
| Perfect match with KRAS Q61H sequence | 604 | −0.95% |
| Perfect match with KRAS Q61L sequence | 405 | −0.64% |
| 1 bp difference from KRAS canonical; random error | 152 | −0.24% |
| 1 bp difference from KRAS canonical; random error | 115 | −0.18% |
| 1 bp difference from KRAS canonical; random error | 114 | −0.18% |
| 1 bp difference from KRAS canonical; random error | 108 | −0.17% |
| 1 bp difference from KRAS canonical; random error | 100 | −0.16% |
| 1 bp difference from KRAS canonical; random error | 93 | −0.15% |
| 1 bp difference from KRAS canonical; random error | 93 | −0.15% |

Table 13a-13c: Detection of KIT SNP Region D816V Mutations

| | | |
|---|---|---|
| Sequence Read by NGS Instrument | # of Reads | % of Total |

13a. Wild-type sample with no mutations; none were detected above the cutoff
Target Name:: KIT SNP Region D816V
Sample Name:: 12878 (KIT canonical)

| | | |
|---|---|---|
| Perfect match with expected KIT Canonical Sequence | 42109 | −85.28% |
| 1 bp difference from KIT canonical; random error | 172 | −0.35% |
| 1 bp difference from KIT canonical; random error | 158 | −0.32% |
| 1 bp difference from KIT canonical; random error | 137 | −0.28% |
| 1 bp difference from KIT canonical; random error | 107 | −0.22% |
| 1 bp difference from KIT canonical; random error | 104 | −0.21% |
| 1 bp difference from KIT canonical; random error | 94 | −0.19% |
| 1 bp difference from KIT canonical; random error | 88 | −0.18% |
| 1 bp difference from KIT canonical; random error | 82 | −0.17% |
| 1 bp difference from KIT canonical; random error | 80 | −0.16% |

13b. Sample is canonical for KIT mutations; none were detected above the cutoff
Target Name:: KIT SNP Region D816V
Sample Name:: HDx3 (wild-type for KIT mutations)

| | | |
|---|---|---|
| Perfect match with expected KIT Canonical Sequence | 36694 | −84.05% |
| 1 bp difference from KIT canonical; random error | 156 | −0.36% |
| 1 bp difference from KIT canonical; random error | 152 | −0.35% |
| 1 bp difference from KIT canonical; random error | 117 | −0.27% |
| 1 bp difference from KIT canonical; random error | 114 | −0.26% |
| 1 bp difference from KIT canonical; random error | 111 | −0.25% |
| 1 bp difference from KIT canonical; random error | 98 | −0.22% |
| 1 bp difference from KIT canonical; random error | 83 | −0.19% |
| 1 bp difference from KIT canonical; random error | 82 | −0.19% |
| 1 bp difference from KIT canonical; random error | 76 | −0.17% |

13c. Sample contains the KIT D816V mutation at 1.3%; it was detected at
greater than 3 standard deviations above cutoff
Target Name:: KIT SNP Region D816V
Sample Name:: HDx7 (KIT D816V mutation at 1.3%)

| | | |
|---|---|---|
| Perfect match with expected KIT Canonical Sequence | 38197 | −84.74% |
| Perfect match with KIT D816V sequence | 427 | −0.95% |
| 1 bp difference from KIT canonical; random error | 167 | −0.37% |
| 1 bp difference from KIT canonical; random error | 149 | −0.33% |
| 1 bp difference from KIT canonical; random error | 112 | −0.25% |
| 1 bp difference from KIT canonical; random error | 107 | −0.24% |
| 1 bp difference from KIT canonical; random error | 92 | −0.20% |
| 1 bp difference from KIT canonical; random error | 84 | −0.19% |
| 1 bp difference from KIT canonical; random error | 82 | −0.18% |
| 1 bp difference from KIT canonical; random error | 82 | −0.18% |

Tables 14a-14c: Detection of EGFR L858R and L861Q Mutations

| | | |
|---|---|---|
| Sequence Read by NGS Instrument | # of Reads | % of Total |

14a. Wild-type sample with no mutations; none detected above the cutoff
Target Name:: EGFR SNPs L858R andL861Q
Sample Name:: 12878 (EGFR canonical)

| | | |
|---|---|---|
| Perfect match with expected EGFR Canonical Sequence | 79758 | −87.22% |
| 1 bp difference from EGFR canonical; random error | 249 | −0.27% |
| 1 bp difference from EGFR canonical; random error | 188 | −0.21% |
| 1 bp difference from EGFR canonical; random error | 164 | −0.18% |
| 1 bp difference from EGFR canonical; random error | 147 | −0.16% |
| 1 bp difference from EGFR canonical; random error | 146 | −0.16% |
| 1 bp difference from EGFR canonical; random error | 145 | −0.16% |
| 1 bp difference from EGFR canonical; random error | 127 | −0.14% |
| 1 bp difference from EGFR canonical; random error | 126 | −0.14% |
| 1 bp difference from EGFR canonical; random error | 125 | −0.14% |

14b. Sample is canonical for EGFR mutations at these codons; none were detected above the cutoff
Target Name:: EGFR SNPs L858R and L861Q
Sample Name:: HDx3 (wild-type for these EGFR mutations)

| | | |
|---|---|---|
| Perfect match with expected EGFR Canonical Sequence | 56190 | −87.29% |
| 1 bp difference from EGFR canonical; random error | 148 | −0.23% |
| 1 bp difference from EGFR canonical; random error | 122 | −0.19% |
| 1 bp difference from EGFR canonical; random error | 115 | −0.18% |
| 1 bp difference from EGFR canonical; random error | 113 | −0.18% |
| 1 bp difference from EGFR canonical; random error | 113 | −0.18% |
| 1 bp difference from EGFR canonical; random error | 105 | −0.16% |
| 1 bp difference from EGFR canonical; random error | 105 | −0.16% |
| 1 bp difference from EGFR canonical; random error | 100 | −0.16% |
| 1 bp difference from EGFR canonical; random error | 99 | −0.15% |

14c. Sample contains both L858R and L861Q mutations; both were
detected at greater than 3 standard deviations above cutoff
Target Name:: EGFR SNPs L858R and L861Q
Sample Name:: HDx7 (EGFR L858R and L861Q @ 1%)

| | | |
|---|---|---|
| Perfect match with expected EGFR Canonical Sequence | 95719 | −86.03% |
| Perfect match with EGFR L858R sequence | 903 | −0.81% |
| Perfect match with EGFR L861Q sequence | 864 | −0.78% |
| 1 bp difference from EGFR canonical; random error | 291 | −0.26% |
| 1 bp difference from EGFR canonical; random error | 273 | −0.25% |
| 1 bp difference from EGFR canonical; random error | 218 | −0.20% |
| 1 bp difference from EGFR canonical; random error | 170 | −0.15% |
| 1 bp difference from EGFR canonical; random error | 170 | −0.15% |
| 1 bp difference from EGFR canonical; random error | 162 | −0.15% |
| 1 bp difference from EGFR canonical; random error | 161 | −0.14% |

Tables 15a-15c: Detection of BRAF Codon 600 Mutations

| | | |
|---|---|---|
| Sequence Read by NGS Instrument | # of Reads | % of Total |

15a. Wild-type sample with no mutations; none detected above the cutoff
Target Name:: BRAF SNPs around V600
Sample Name:: 12878 (wild-type)

| | | |
|---|---|---|
| Perfect match with expected BRAF Canonical Sequence | 55520 | −89.12% |
| 1 bp difference from BRAF canonical; random error | 163 | −0.26% |
| 1 bp difference from BRAF canonical; random error | 114 | −0.18% |
| 1 bp difference from BRAF canonical; random error | 108 | −0.17% |
| 1 bp difference from BRAF canonical; random error | 107 | −0.17% |
| 1 bp difference from BRAF canonical; random error | 107 | −0.17% |
| 1 bp difference from BRAF canonical; random error | 95 | −0.15% |
| 1 bp difference from BRAF canonical; random error | 91 | −0.15% |
| 1 bp difference from BRAF canonical; random error | 88 | −0.14% |
| 1 bp difference from BRAF canonical; random error | 87 | −0.14% |

15b. Sample contains 2 BRAF mutations at 4 and 8%; both were detected at
greater than 3 standard deviations above cutoff
Target Name:: BRAF SNPs around V600
Sample Name:: HDx3 (BRAF V600M @ 4% and V600E @ 8%)

| | | |
|---|---|---|
| Perfect match with expected BRAF Canonical Sequence | 32528 | −80.43% |
| Perfect match with BRAF V600E sequence | 2513 | −6.21% |
| Perfect match with BRAF V600M sequence | 1093 | −2.70% |
| 1 bp difference from BRAF canonical; random error | 115 | −0.28% |
| 1 bp difference from BRAF canonical; random error | 91 | −0.23% |
| 1 bp difference from BRAF canonical; random error | 88 | −0.22% |
| 1 bp difference from BRAF canonical; random error | 68 | −0.17% |
| 1 bp difference from BRAF canonical; random error | 58 | −0.14% |
| 1 bp difference from BRAF canonical; random error | 54 | −0.13% |
| 1 bp difference from BRAF canonical; random error | 50 | −0.12% |

15c. Sample contains 5 BRAF mutations ranging from 1-8%; 4 were detected at greater
than 3 standard deviations above cutoff, 1 at greater than 1 standard deviation
Target Name:: BRAF SNPs around V600
Sample Name:: HDx7 (BRAF V600E @ 8%; V600G, V600K, V600M and V600R @ 1%)

| | | |
|---|---|---|
| Perfect match with expected BRAF Canonical Sequence | 49543 | −80.12% |
| Perfect match with BRAF V600E sequence | 4306 | −6.96% |
| Perfect match with BRAF V600G sequence | 536 | −0.87% |
| Perfect match with BRAF V600K sequence | 447 | −0.72% |
| Perfect match with BRAF V600M sequence | 314 | −0.51% |
| Perfect match with BRAF V600R sequence | 189 | −0.31% |
| 1 bp difference from BRAF canonical; random error at cutoff | 170 | −0.27% |
| 1 bp difference from BRAF canonical; random error | 115 | −0.19% |
| 1 bp difference from BRAF canonical; random error | 90 | −0.15% |
| 1 bp difference from BRAF canonical; random error | 87 | −0.14% |

b) Medium Mutations

The Cancer Test was used to detect insertions or deletions in target regions in the EGFR, PTEN and FLT3 genes.

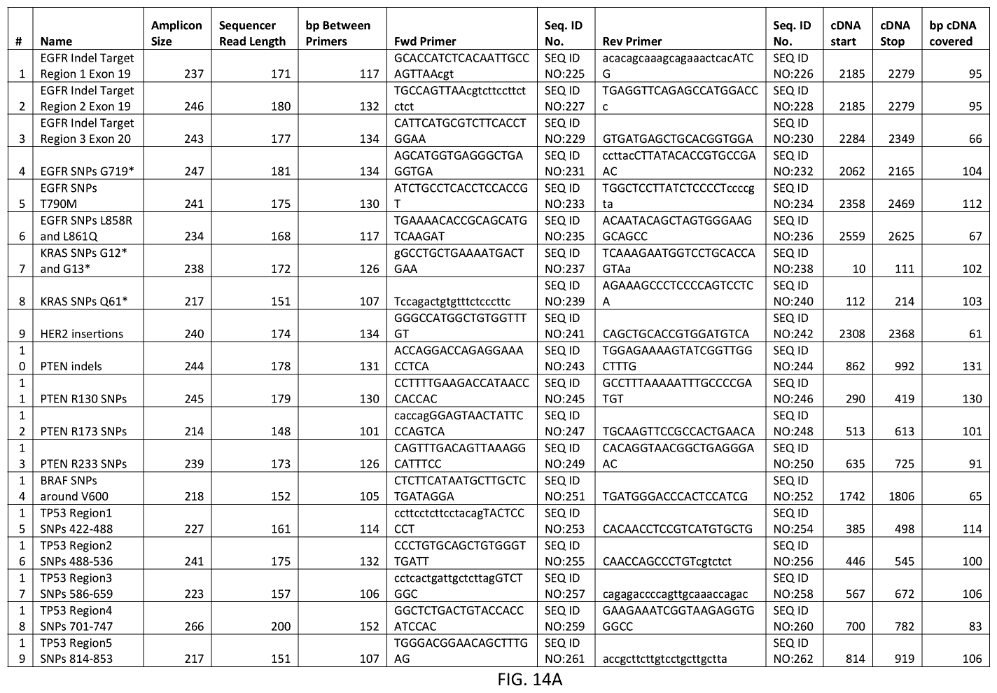

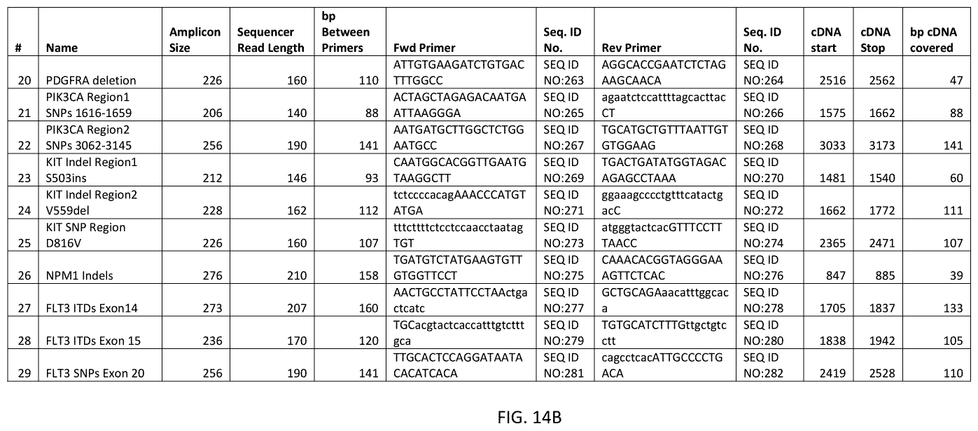

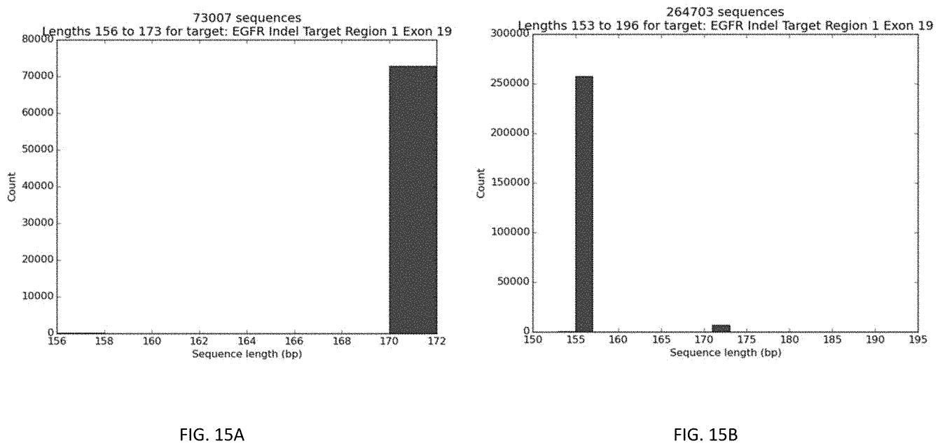

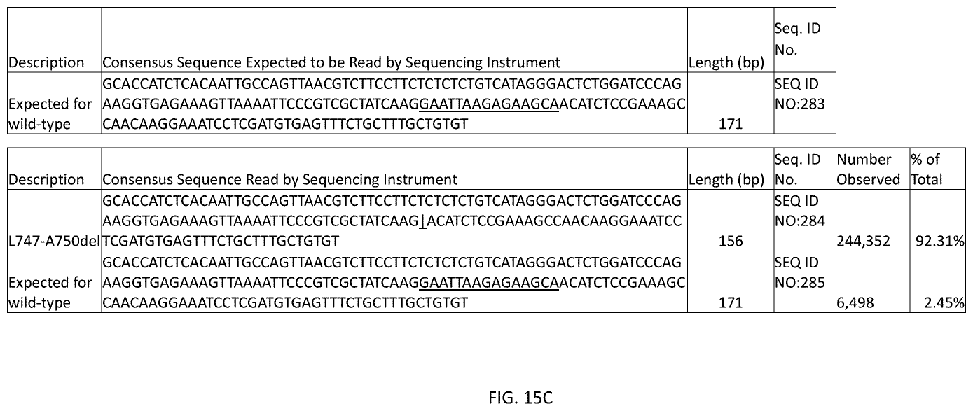

The results for this EGFR target amplicon are shown in FIGS. 15A-15C for the cancer cell line sample HCC 827 which is know to contain the mutations EGFR L747-A750del, a 15 base-pair (bp) deletion in exon 19 of EGFR. FIGS. 15A and B show the distribution of sequence read lengths for this amplicon. For wild-type samples (FIG. 15A), reads of this amplicon are expected to be 171 bp. For the deletion sample (FIG. 15B) 250,000 (˜93%) of the sequence reads for this amplicon were 156 bp long, exactly 15 bp shorter than the 171 bp expected for wild-type. FIG. 15C shows the sequence that is expected to be read by the sequencer followed by what is actually read by the sequencer. The number observed is the number of reads that exactly aligned to the sequence shown in the table. In this case 244,352 reads aligned perfectly to the sequence shown that lacks the 15 bp show in red in the reference. The location of the deletion is depicted by a vertical red bar in the L747-A750del reads.

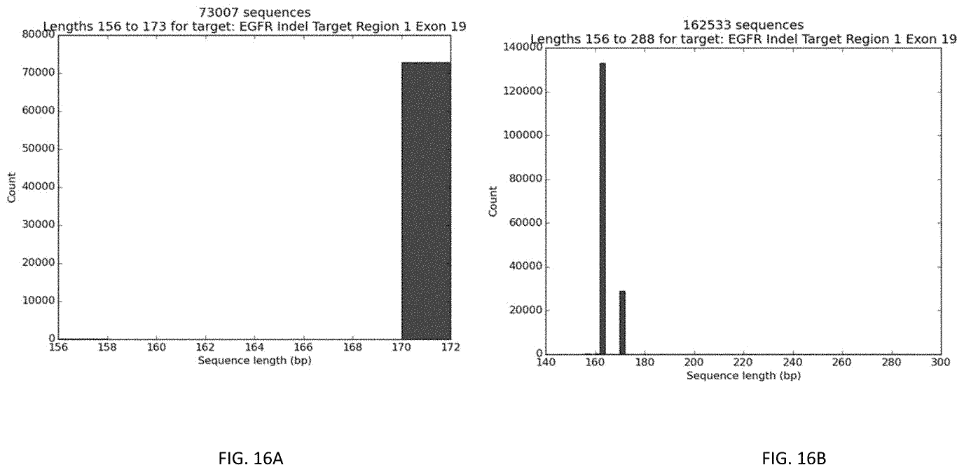

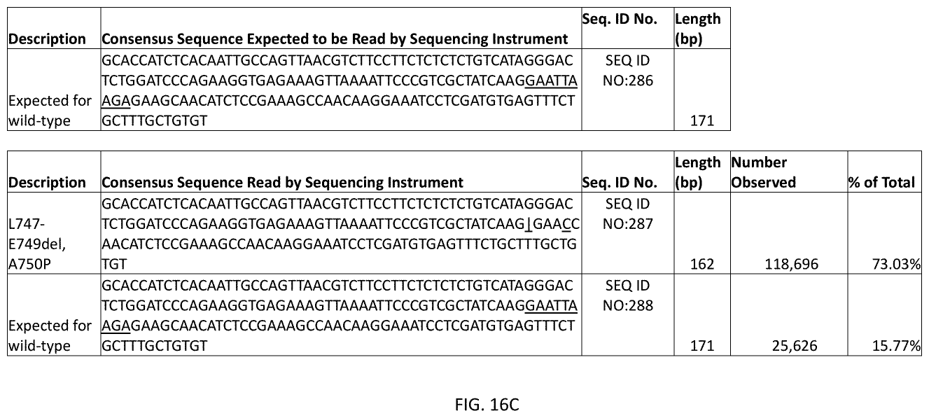

The results for this EGFR target amplicon are shown in FIGS. 16A-16C for the cancer cell line sample HCCC4006 which is know to contain the mutations EGFR L747-E749del and A750P, a 9 base pair deletion followed by a G to C substitution 4 base-pairs after the deletion. FIGS. 16A and 16B show the distribution of sequence read lengths for this amplicon. For wild-type samples (FIG. 16A), reads of this amplicon are expected to be 171 bp. For mutant sample (FIG. 16B) 118,696 (˜73%) of the sequence reads for this amplicon were 162 bp long, exactly 9 bp shorter than the 171 bp expected for wild-type. FIG. 16C shows the sequence that is expected to be read by the sequencer followed by what is actually read by the sequencer. The 9 bases deleted from the canonical reference are shown in read. In the L747-E749del, A750P reads the point of the deletion is depicted by a vertical red bar and the G>C SNP is shown in red as well.

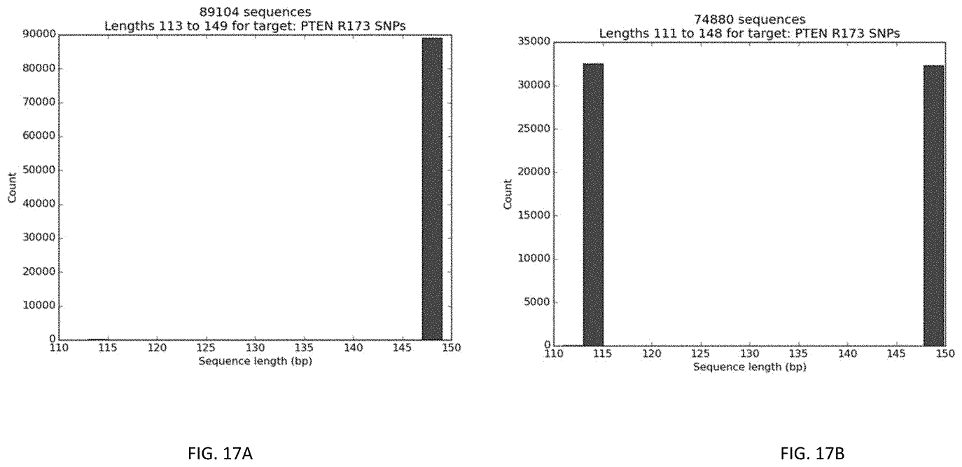

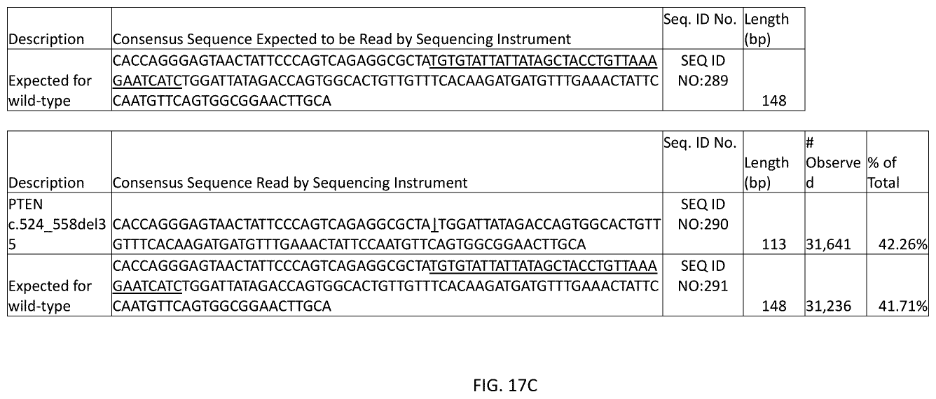

The results for this PTEN target amplicon are shown in FIGS. 17A-17C for the cancer cell line sample A2058 which is know to contain the mutations PTEN c.524_558del35, a 35 base-pair (bp) deletion. FIGS. 17A and 17B show the distribution of sequence read lengths for this amplicon. For wild-type samples (FIG. 17A), reads of this amplicon are expected to be 148 bp. For deletion sample (FIG. 17B) 33,000 (˜44%) of the sequence reads for this amplicon were 113 bp long, exactly 35 bp shorter than the 148 bp expected for wild-type. FIG. 17C shows the sequence that is expected to be read by the sequencer followed by what is actually read by the sequencer. The number observed is the number of reads that exactly aligned to the sequence shown in the table. In this case 31,641 reads aligned perfectly to the sequence shown that lacks the 35 bp show in red in the reference. The location of the deletion is depicted by a vertical red bar in the PTEN c.524_558del35 reads.

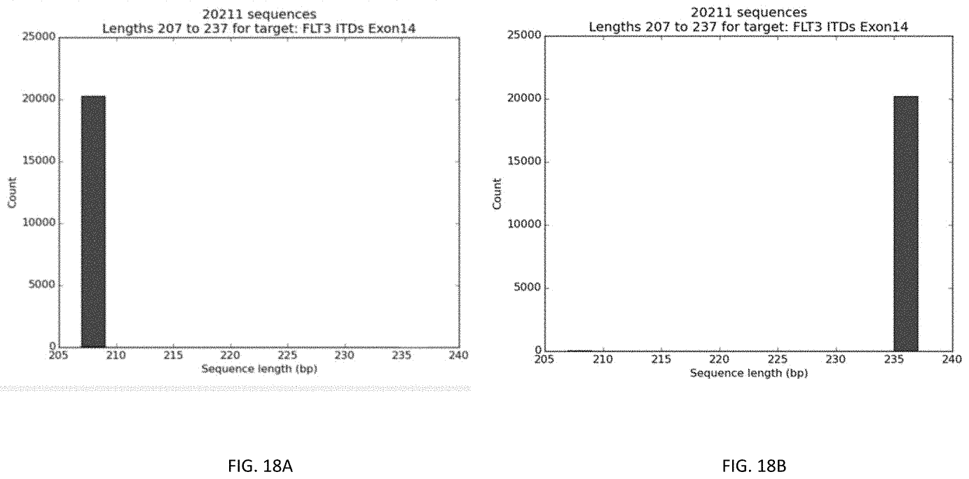

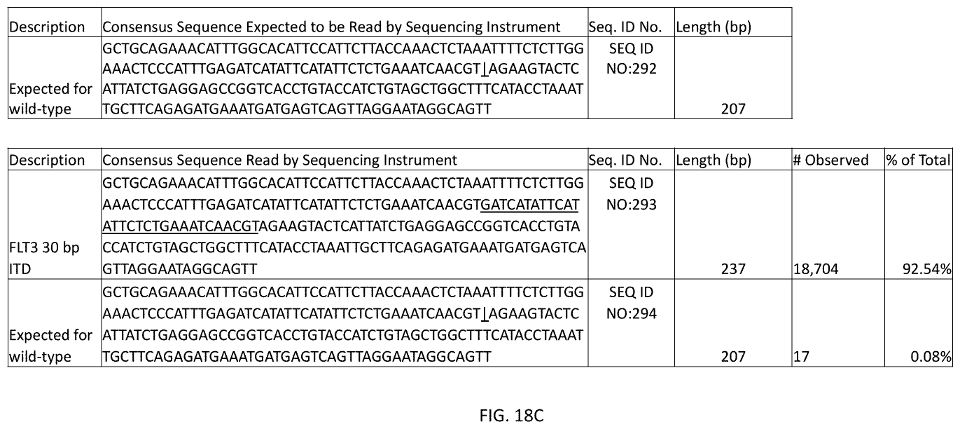

The results for this FLT3 target amplicon are shown in FIGS. 18A-18C for the cancer cell line sample MV-4-11 which is know to contain the mutation a 30 base-pair (bp) FLT3 ITD insertion. FIGS. 18A and 18B show the distribution of sequence read lengths for this amplicon. For wild-type samples (FIG. 18A), reads of this amplicon are expected to be 207 bp. For insertion sample (FIG. 18B) 18,000 (˜93%) of the sequence reads for this amplicon were 237 bp long, exactly 30 bp longer than the 207 bp expected for wild-type. FIG. 18C shows the sequence that is expected to be read by the sequencer followed by what is actually read by the sequencer. The number observed is the number of reads that exactly aligned to the sequence shown in the table. In this case 18,704 reads aligned perfectly to the sequence with the 30 bp insertion shown in red. The inserted sequence is the exact duplicate of the 30 bp that precedes it in the read, as is generally the case with FLT3 insertion mutations. The location in the reference where the insertion occurs is depicted by a vertical red bar.

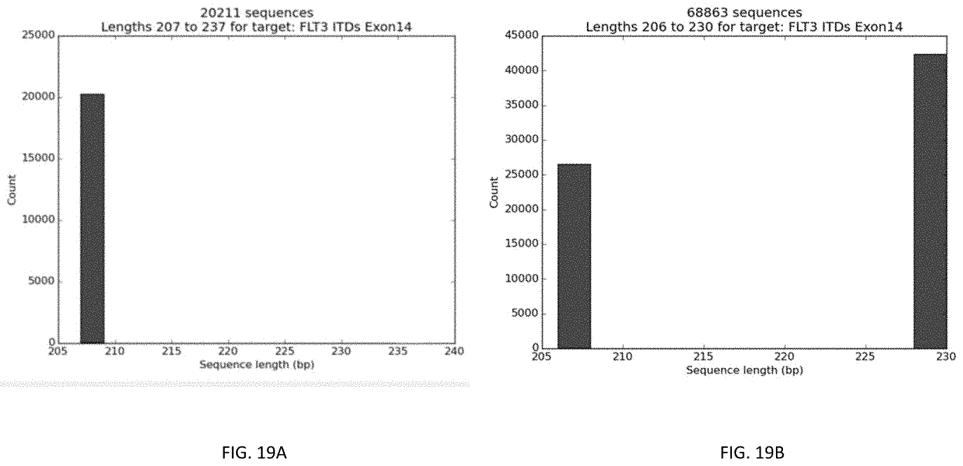

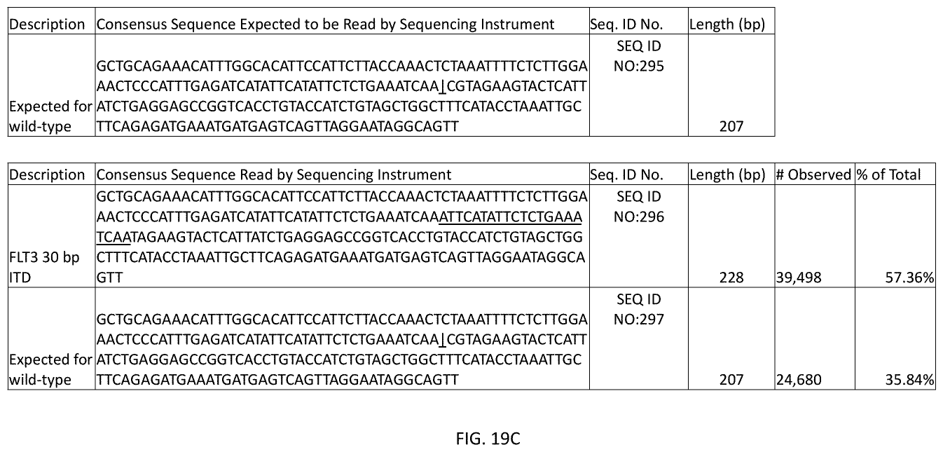

The results for this FLT3 target amplicon are shown in FIGS. 19A-19C for the cancer cell line sample MOLM-13 which is known to contain the mutation a 21 base-pair (bp) FLT3 ITD insertion. FIGS. 19A and 19B the distribution of sequence read lengths for this amplicon. For wild-type samples (FIG. 19A), reads of this amplicon are expected to be 207 bp. For the insertion sample (FIG. 19B) 39,498 (about 57%) of the sequence reads for this amplicon were 228 bp long, exactly 21 bp longer than the 207 bp expected for wild-type. FIG. 19C shows the sequence that is expected to be read by the sequencer followed by what is actually read by the sequencer. The number observed is the number of reads that exactly aligned to the sequence shown in the table. In this case 39,498 reads aligned perfectly to the sequence with the 21 bp insertion shown in red. The inserted sequence is the exact duplicate of the 21 bp that precedes it in the read, as is generally the case with FLT3 insertion mutations. The location in the reference where the insertion occurs is depicted by a vertical red bar.

### REFERENCES

1. Mardis, Elaine R. “A decade/'s perspective on DNA sequencing technology.” Nature 470.7333 (2011): 198-203.
2. Sanger, F., S. Nicklen, and A. R. Coulson. 1977. DNA sequencing with chain-terminating inhibitors. Proc. Natl. Acad. Sci. USA 12:5463-5467.
3. Lander, Eric S., et al. “Initial sequencing and analysis of the human genome.” Nature 409.6822 (2001): 860-921.
4. Collins, F. S., et al. “Finishing the euchromatic sequence of the human genome.” Nature 431.7011 (2004): 931-945.
5. Katsnelson, A. “Human genome: genomes by the thousand.” Nature 467 (2010): 1026-1027.
6. Roukos, D. H. “Trastuzumab and beyond: sequencing cancer genomes and predicting molecular networks.” The pharmacogenomics journal 11.2 (2010): 81-92.
7. Worthey, Elizabeth A., et al. “Making a definitive diagnosis: successful clinical application of whole exome sequencing in a child with intractable inflammatory bowel disease.” Genetics in medicine 13.3 (2010): 255-262.
8. Mantovani, Giovanna, et al. “Pseudohypoparathyroidism and GNAS epigenetic defects: clinical evaluation of Albright hereditary osteodystrophy and molecular analysis in 40 patients.” Journal of Clinical Endocrinology & Metabolism 95.2 (2010): 651-658.
9. Adib-Samii, Poneh, et al. “Clinical Spectrum of CADASIL and the Effect of Cardiovascular Risk Factors on Phenotype Study in 200 Consecutively Recruited Individuals.” Stroke 41.4 (2010): 630-634.
10. Yeo, Zhen Xuan, et al. “Improving Indel Detection Specificity of the Ion Torrent PGM Benchtop Sequencer.” PloS one 7.9 (2012): e45798.
11. Albers, Cornelis A., et al. “Dindel: accurate indel calls from short-read data.” Genome research 21.6 (2011): 961-973.
12. Grimm, Dominik, et al. “Accurate indel prediction using paired-end short reads.” BMC genomics 14.1 (2013): 1-10.
13. Shigemizu, Daichi, et al. “A practical method to detect SNVs and indels from whole genome and exome sequencing data.” Scientific reports 3 (2013).
14. Alkan, Can, Bradley P. Coe, and Evan E. Eichler. “Genome structural variation discovery and genotyping.” Nature Reviews Genetics 12.5 (2011): 363-376.
15. Rosen, Shara. Wold Market for Personalized Medicine. New York, N.Y.: Kalorama Information, 2012. Industry Report.
16. Pfizer Inc. Xalkori (Crizotinib). [Online] [Dec. 12, 2012.] http://www.xalkori.com/.
17. D, Shibaia. Mutation and epi genetic molecular clocks in cancer. Carcinogenesis. 32, 2011, Vols. 123-128.
18. McMahon M A, et al. The HBV drug entecavir—effects on HIV-1 replication and resistance. N Engl J Med. 356, 2007, Vols. 2614-2621.
19. Eastman P S, et al. Maternal viral genotypic zidovudine resistance and infrequent failure of zidovudine therapy to prevent perinatal transmission of human immunodeficiency virus type 1 in pediatric AIDS Clinical Trials Group Protocol 076. J Infect Dis. 177, 1998, Vols. 557-564.
20. Chiu R W, e. a. (2008). Noninvasive prenatal diagnosis of fetal chromosomal aneuploidy by massively parallel genomic sequencing of DNA in maternal plasma. Proc Natl Acad Sci, 20458-20463.
21. Fan H C, B. Y. (2008). Noninvasive diagnosis of fetal aneuploidy by shotgun sequencing DNA from maternal blood. Proc Natl Acad Sci, 16266-16271.
22. Hoque M O, e. a. (2003). High-throughput molecular analysis of urine sediment for the detection of bladder cancer by high-density single-nucleotide polymorphism array. Cancer Res, 5723-5726.
23. F B, Thunnissen. (2003). Sputum examination for early detection of lung cancer. J Clin Pathol, 805-810.
24. Diehl F, e. a. (2008). Analysis of mutations in DNA isolated from plasma and stool of colorectal cancer patients. Gastroenterology, 489-498.
25. Quail M A, e. a. (2008). A large genome center's improvements to the Illumina sequencing system. Nat Methods, 1005-1010.
26. Nazarian R, e. a. (2010). Melanomas acquire resistance to B-RAF(V600E) inhibition by RT or N-RAS upregulation. Nature, 973-977.
27. He Y, e. a. (2010). Heteroplasmic mitochondrial DNA mutations in normal and tumour cells. Nature, 610-614.
28. Gore A, e. a. (2011). Somatic coding mutations in human induced pluripotent stem cells. Nature, 63-67.
29. Dohm J C, L. C. (2008). Substantial biases in ultrashort read data sets from high-throughput DNA sequencing. Nucleic Acids Res, 05.
30. Erlich Y, M. P. (2008). Alta-Cyclic: a self-optimizing base caller for next-generation sequencing. Nature Methods, 679-682.
31. Rougemont J, e. a. (2008). Probabilistic base calling of Solexa sequencing data. BMC Bioinformatics, 431.
32. Druley T E, e. a. (2009). Quantification of rare allelic variants from pooled genomic DNA. Nature Methods, 263-265.
33. Vallania, Francesco L M, et al. “High-throughput discovery of rare insertions and deletions in large cohorts.” Genome research 20.12 (2010): 1711-1718.
34. Lovly, C., L. Horn, W. Pao. 2012. KRAS Mutations in Non-Small Cell Lung Cancer (NSCLC). My Cancer Genome at www.mycancergenome.org/content/disease/lung-cancer/kras/
35. De Roock, W., et al. (2007) KRAS mutations preclude tumor shrinkage of colorectal cancers treated with cetuximab. J. Clin. Oncol. 25 (18S), 4132.
36. Davies, Helen, et al. “Mutations of the BRAF gene in human cancer.” Nature 417.6892 (2002): 949-954.
37. Maldonado, Janet L., et al. “Determinants of BRAF mutations in primary melanomas.” Journal of the National Cancer Institute 95.24 (2003): 1878-1890.
38. Chapman, Paul B., et al. “Improved survival with vemurafenib in melanoma with BRAF V600E mutation.” New England Journal of Medicine 364.26 (2011): 2507-2516.
39. Lynch, Thomas J., et al. “Activating mutations in the epidermal growth factor receptor underlying responsiveness of non-small-cell lung cancer to gefitinib.” New England Journal of Medicine 350.21 (2004): 2129-2139.
40. Mitsudomi, Tetsuya, and Yasushi Yatabe. “Epidermal growth factor receptor in relation to tumor development: EGFR gene and cancer.” FEBS journal 277.2 (2010): 301-308.
41. Kobayashi, Susumu, et al. “EGFR mutation and resistance of non-small-cell lung cancer to gefitinib.” New England Journal of Medicine 352.8 (2005): 786-792.
42. Pao, William, et al. “Acquired resistance of lung adenocarcinomas to gefitinib or erlotinib is associated with a second mutation in the EGFR kinase domain.” PLoS medicine 2.3 (2005): e73.
43. Antonescu, Cristina R., et al. “L576P KIT mutation in anal melanomas correlates with KIT protein expression and is sensitive to specific kinase inhibition.” International journal of cancer 121.2 (2007): 257-264.
44. Beadling, Carol, et al. “KIT gene mutations and copy number in melanoma subtypes.” Clinical Cancer Research 14.21 (2008): 6821-6828.
45. Curtin, John A., et al. “Somatic activation of KIT in distinct subtypes of melanoma.” Journal of clinical oncology 24.26 (2006): 4340-4346.
46. Growney, Joseph D., et al. “Activation mutations of human c-KIT resistant to imatinib mesylate are sensitive to the tyrosine kinase inhibitor PKC412.” Blood 106.2 (2005): 721-724.
47. Paez, J. Guillermo, et al. “EGFR mutations in lung cancer: correlation with clinical response to gefitinib therapy.” Science 304.5676 (2004): 1497-1500.
48. Pao, William, et al. “EGF receptor gene mutations are common in lung cancers from “never smokers” and are associated with sensitivity of tumors to gefitinib and erlotinib.” Proceedings of the National Academy of Sciences of the United States of America 101.36 (2004): 13306-13311.
49. Maemondo, Makoto, et al. “Gefitinib or chemotherapy for non-small-cell lung cancer with mutated EGFR.” New England Journal of Medicine 362.25 (2010): 2380-2388.
50. Rosell, Rafael, et al. “Erlotinib versus standard chemotherapy as first-line treatment for European patients with advanced EGFR mutation-positive non-small-cell lung cancer (EURTAC): a multicentre, open-label, randomised phase 3 trial.” The lancet oncology 13.3 (2012): 239-246.
51. Yuza, Yuki, et al. “Allele-dependent variation in the relative cellular potency of distinct EGFR inhibitors.” CANCER BIOLOGY AND THERAPY 6.5 (2007): 661.
52. Shigematsu, Hisayuki, et al. “Somatic mutations of the HER2 kinase domain in lung adenocarcinomas.” Cancer research 65.5 (2005): 1642-1646.
53. Buttitta, Fiamma, et al. “Mutational analysis of the HER2 gene in lung tumors from Caucasian patients: mutations are mainly present in adenocarcinomas with bronchioloalveolar features.” International journal of cancer 119.11 (2006): 2586-2591.
54. Arcila, Maria E., et al. “Prevalence, clinicopathologic associations, and molecular spectrum of ERBB2 (HER2) tyrosine kinase mutations in lung adenocarcinomas.” Clinical Cancer Research 18.18 (2012): 4910-4918.
55. Wang, Shizhen Emily, et al. “HER2 kinase domain mutation results in constitutive phosphorylation and activation of HER2 and EGFR and resistance to EGFR tyrosine kinase inhibitors.” Cancer cell 10.1 (2006): 25-38.
56. Mazieres, Julien, et al. “Lung cancer that harbors an HER2 mutation: epidemiologic characteristics and therapeutic perspectives.” Journal of Clinical Oncology 31.16 (2013): 1997-2003.
57. Gatzemeier, U., et al. “Randomized phase II trial of gemcitabine-cisplatin with or without trastuzumab in HER2-positive non-small-cell lung cancer.” Annals of Oncology 15.1 (2004): 19-27.
58. Langer, Corey J., et al. “Trastuzumab in the treatment of advanced non-small-cell lung cancer: is there a role? Focus on Eastern Cooperative Oncology Group study 2598.” Journal of clinical oncology 22.7 (2004): 1180-1187.
59. Patel, Jay P., et al. “Prognostic relevance of integrated genetic profiling in acute myeloid leukemia.” New England Journal of Medicine 366.12 (2012): 1079-1089.
60. Estey, Elihu H. “Acute myeloid leukemia: 2012 update on diagnosis, risk stratification, and management.” American journal of hematology 87.1 (2012): 89-99.
61. Dohner, Hartmut, et al. “Diagnosis and management of acute myeloid leukemia in adults: recommendations from an international expert panel, on behalf of the European LeukemiaNet.” Blood 115.3 (2010): 453-474.
62. Man, Cheuk Him, et al. “Sorafenib treatment of FLT3-ITD+ acute myeloid leukemia: favorable initial outcome and mechanisms of subsequent nonresponsiveness associated with the emergence of a D835 mutation.” Blood 119.22 (2012): 5133-5143.
63. Smith, C. C., and N. P. Shah. “The role of kinase inhibitors in the treatment of patients with acute myeloid leukemia.” American Society of Clinical Oncology educational book/ASCO. American Society of Clinical Oncology. Meeting. Vol. 2013. 2012.
64. Gnirke, Andreas, et al. “Solution hybrid selection with ultra-long oligonucleotides for massively parallel targeted sequencing.” Nature biotechnology 27.2 (2009): 182-189.
65. Camidge, D. Ross, et al. “Activity and safety of crizotinib in patients with ALK-positive non-small-cell lung cancer: updated results from a phase 1 study.” The lancet oncology 13.10 (2012): 1011-1019.
66. Kim, Dong-Wan, et al. “Ceritinib in advanced anaplastic lymphoma kinase (ALK)-rearranged (ALK+) non-small cell lung cancer (NSCLC): Results of the ASCEND-1 trial.” ASCO Annual Meeting Proceedings. Vol. 32. No. 15_suppl. 2014.

The relevant teachings of all patents, published applications and references cited herein are incorporated by reference in their entirety.

While this invention has been particularly shown and described with references to example embodiments thereof, it will be understood by those skilled in the art that various changes in form and details may be made therein without departing from the scope of the invention encompassed by the appended claims.

1. A method for detecting a genetic mutation, comprising the steps of:
a) obtaining a plurality of target nucleotide sequences from the products of one or more nucleic acid amplification reactions;
b) sorting the target nucleotide sequences into a plurality of bins according to a sorting criterion;
c) assigning a unique set of reference nucleotide sequences to each bin, wherein the reference nucleotide sequences include non-canonical reference sequences;
d) aligning the target nucleotide sequences in each bin with the set of reference nucleotide sequences assigned to the bin;
e) quantifying the number of target nucleotide sequences in a bin that align with each non-canonical reference sequence; and
f) detecting a genetic mutation by:
1) identifying a target nucleotide sequence that aligns with a non-canonical reference sequence in a bin,
2) identifying a target nucleotide sequence that is present in an unexpected bin, or
3) identifying the absence of target nucleotide sequences in an expected bin.

2-18. (canceled)

19. The method of claim 1, wherein the plurality of bins include a bin comprising a rearrangement hash of reference nucleotide sequences

20. The method of claim 1, wherein the plurality of bins includes a bin comprising a SNP hash of reference nucleotide sequences, a bin comprising an indel hash of reference nucleotide sequences and a bin comprising a rearrangement hash of reference nucleotide sequences.

21. The method of claim 1, wherein the unique set of reference nucleotide sequences in each bin comprises more than 100 different reference nucleotide sequences.

22. (canceled)

23. The method of claim 1, wherein the background number is determined by quantifying the number of target nucleotide sequences that align with each reference sequence.

24. (canceled)

25. The method of claim 1, wherein the genetic mutation is a germline mutation.

26. The method of claim 1, wherein the genetic mutation is a somatic mutation.

27-30. (canceled)

31. The method of claim 1, wherein the target nucleotide sequences are from a nucleic acid molecule obtained from a biological tissue sample.

32-34. (canceled)

35. An apparatus for detecting a genetic mutation, comprising a processor configured to:
a) receive sequence data comprising a plurality of target nucleotide sequences;
b) sort the target nucleotide sequences into a plurality of bins according to a sorting criterion;
c) generate and assign a unique set of reference nucleotide sequences to each bin, wherein the reference nucleotide sequences include non-canonical reference sequences;
d) align the target nucleotide sequences in each bin with the set of reference nucleotide sequences assigned to the bin;
e) quantify the number of target nucleotide sequences in a bin that align with each non-canonical reference sequence; and
f) provide a user output indicating whether a genetic mutation is present in the target nucleotide sequence.

36-42. (canceled)

43. A method for detecting the presence of a genetic mutation that alters gene expression, comprising:
a) obtaining a plurality of target nucleotide sequences;
b) aligning the target nucleotide sequences with a set of reference nucleotide sequences comprising a first reference sequence and at least one additional reference sequence;
c) quantifying the number of target nucleotide sequences that align with each of the reference nucleotide sequences; and
d) comparing the quantity of target nucleotide sequences that align with the first reference nucleotide sequence to the quantity of target nucleotide sequences that align with the other reference nucleotide sequences,

44. The method of claim 43, wherein an increase or decrease in the quantity of target nucleotide sequences that align with the first reference nucleotide sequence relative to the quantity of target nucleotide sequences that align with the other reference nucleotide sequences is indicative of a genetic mutation that alters gene expression.

45. The method of claim 43, wherein the genetic mutation is a structural variation involving the rearrangement, deletion, insertion or repetition of about 50 to 25,000 base pairs.

46. The method of claim 43, wherein the genetic mutation is a copy-number-variation involving the rearrangement, deletion, insertion or repetition of 25,001 to 250,000,000 base pairs.

47. The method of claim 43, wherein the genetic mutation increases the expression of an RNA transcript.

48. The method of claim 43, wherein the genetic mutation decreases the expression of an RNA transcript.

49. The method of claim 43, wherein the target nucleotide sequences are generated by a sequencer.

50-53. (canceled)

54. The method of claim 43, wherein the nucleic acid amplification reaction is a multiplex PCR reaction, a single-plex PCR reaction or a combination thereof.

55. A method for detecting a genetic mutation, comprising:
a) amplifying three or more target nucleotide sequences in a sample comprising genomic DNA, wherein:
1) at least one target nucleotide sequence is being analyzed for a single nucleotide polymorphism (SNP),
2) at least one target nucleotide sequence is being analyzed for an insertion, a deletion, or an insertion and a deletion, and
3) at least one target nucleotide sequence is being analyzed for a rearrangement,
thereby producing an amplicon for each target nucleotide sequence;
b) sequencing the amplicons produced in a); and
c) analyzing the sequences of the amplicons for the presence of a genetic mutation.

56-59. (canceled)

60. The method of claim 55, wherein the first amplification reaction is performed using a different pair of target-specific primers for each target nucleotide sequence, and at least one primer in each pair includes an adapter.

61-77. (canceled)

78. A kit for detecting a genetic mutation, comprising:
a) a first probe set comprising:
1) a pair of target-specific primers for detecting a single nucleotide polymorphism (SNP) in at least one target nucleotide sequence,
2) a pair of target-specific primers for detecting an insertion, a deletion, or an insertion and a deletion in at least one target nucleotide sequence, and
3) a pair of target-specific primers for detecting a rearrangement in at least one target nucleotide sequence; and
b) a second probe set comprising sequencer-specific primers.

79-89. (canceled)
